(19) United States
(12) Patent Application Publication
(10) Pub. No.: US 2026/0223852 A1
(43) Pub. Date: Aug. 6, 2026

(54) FUNGICIDAL COMPOSITIONS

(71) Applicant: SYNGENTA CROP PROTECTION AG, Basel (CH)

(72) Inventors: Andrew EDMUNDS, Stein (CH); Christopher Charles SCARBOROUGH, Stein (CH); Hanno Christian WOLF, Stein (CH); Valeria GRASSO, Stein (CH)

(73) Assignee: SYNGENTA CROP PROTECTION AG, Basel (CH)

(21) Appl. No.: 19/152,825

(22) PCT Filed: Jan. 30, 2024
(86) PCT No.: PCT/EP2024/052205

§ 371 Date: Jul. 31, 2025

(30) Foreign Application Priority Data
Feb. 1, 2023 (EP) 23154427.1
Jun. 21, 2023 (EP) 23180646.4

(51) Int. Cl. A01N 43/80; A01N 43/82; A01P 3/00; C07D 413/14; C07D 417/14

(52) U.S. Cl. CPC A01N 43/80; A01N 43/82; A01P 3/00; C07D 413/14; C07D 417/14

(57) ABSTRACT

A fungicidal composition comprising a mixture of components (A) and (B), wherein components (A) and (B) are as defined in claim 1, and use of the compositions in agriculture or horticulture for controlling or preventing infestation of plants by phytopathogenic microorganisms, preferably fungi.

The present invention relates to novel fungicidal compositions, to their use in agriculture or horticulture for controlling diseases caused by phytopathogens, especially phytopathogenic fungi, and to methods of controlling diseases on useful plants.

Whilst many fungicidal compounds and compositions, belonging to various different chemical classes, have and are being developed for use as fungicides in crops of useful plants, crop tolerance and activity against particular phytopathogenic fungi do not always satisfy the needs of agricultural practice in many respects. Therefore, there is a continuing need to find new compounds and compositions having superior biological properties for use in controlling or preventing infestation of plants by phytopathogenic fungi. For example, compounds possessing a greater biological activity, an advantageous spectrum of activity, an increased safety profile, improved physico-chemical properties, increased biodegradability. Or else, compositions possessing a broader spectrum of activity, improved crop tolerance, synergistic interactions or potentiating properties, or compositions which display a more rapid onset of action or which have longer lasting residual activity or which enable a reduction in the number of applications and/or a reduction in the application rate of the compounds and compositions required for effective control of a phytopathogen, thereby enabling beneficial resistance-management practices, reduced environmental impact and reduced operator exposure.

The use of compositions comprising mixtures of different fungicidal compounds possessing different modes of action can address some of these needs (e.g., by combining fungicides with differing spectrums of activity).

According to the present invention, there is provided a fungicidal composition comprising a mixture of components (A) and (B) as active ingredients, wherein component (A) is a compound of formula (I):

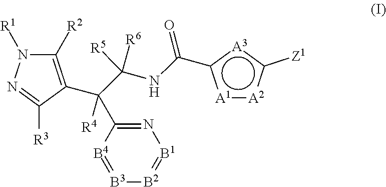

wherein
R¹ is selected from C₁-C₄-alkyl;
R² is selected from hydrogen, halogen, or C₁-C₄-alkyl;
R³ is selected from hydrogen or C₁-C₄-alkyl;
R⁴ is selected from C₁-C₄-alkyl;
R⁵ and R⁶ are independently selected from hydrogen or C₁-C₄-alkyl;
A¹, A² and A³ are independently selected from CR⁷, N, NR⁸, O, or S, with the proviso that at least one of A¹,
A² and A³ is selected from N, O or S, and that no more than one of A¹, A² and A³ is O or S;
R⁷ and R⁸ are independently selected from hydrogen, or C₁-C₄-alkyl;
B¹ is CR⁹ or N, B² is CR¹⁰ or N, B³ is CR¹¹ or N, B⁴ is CR¹² or N, with the proviso that only one of B¹, B², B³, and B⁴ is N;
R⁹, R¹⁰, R¹¹ and R¹² are independently selected from hydrogen, halogen, cyano, C₁-C₄-alkyl, C₁-C₄-alkoxy, C₁-C₄-haloalkyl, C₁-C₄-haloalkoxy, C₁-C₄-alkoxy-C₁-C₄ alkyl, C₁-C₄-alkoxycarbonyl, C₁-C₄-alkylcarbonyl, N—C₁-C₄ alkoxy-C₁-C₄-alkyl-carbonimidoyl, N-hydroxy-C₁-C₄-alkyl-carbonimidoyl, or C₃-C₆-cycloalkyl, wherein said C₃-C₆-cycloalkyl is unsubstituted or substituted by 1 or 2 substituents independently selected from halogen, cyano, C₁-C₄-alkyl, C₁-C₄-haloalkyl, or C₁-C₄-alkoxy; and
Z¹ is selected from C₁-C₄-alkyl, phenyl, pyridyl, or C₃-C₆-cycloalkyl, wherein any of said phenyl, pyridyl and C₃-C₆-cycloalkyl are unsubstituted or substituted by 1 or 2 substituents independently selected from halogen, cyano, C₁-C₄-alkyl, C₁-C₄-haloalkyl, C₁-C₄-alkoxy, or C₁-C₄-haloalkoxy;
or salt or N-oxide thereof,
and
component (B) is a compound selected from the group consisting of:
pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidine, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]-quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethyl-cyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)-amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

The term “compounds of formula (I)” refers to component A.

In general, the weight ratio of component (A) to component (B) may preferably be from 100:1 to 1:100, from 50:1 to 1:50, from 20:1 to 1:40, from 15:1 to 1:30, from 12:1 to 1:25, from 10:1 to 1:20, from 5:1 to 1:15, from 3:1 to 1:10 or from 2:1 to 1:5.

Further according to the invention, there is provided a method of controlling or preventing phytopathogenic diseases, especially phytopathogenic fungi, on useful plants or on propagation material thereof, which comprises applying to the useful plants, the locus thereof or propagation material thereof a fungicidal composition according to the invention.

The benefits provided by certain fungicidal mixture compositions according to the invention may also include, inter alia, advantageous levels of biological activity for protecting plants against diseases that are caused by fungi or superior properties for use as agrochemical active ingredients (for example, greater biological activity, an advantageous spectrum of activity, an increased safety profile, improved physico-chemical properties, or increased biodegradability).

The presence of one or more possible asymmetric carbon atoms in a compound of formula (I) means that the compounds may occur in optically isomeric forms, i.e., enantiomeric or diastereomeric forms. Also, atropisomers may occur as a result of restricted rotation about a single bond. The present invention includes all those possible isomeric forms (e.g., geometric isomers) and mixtures thereof for a compound of formula (I).

The present invention includes all possible tautomeric forms for a compound of formula (I), and also a racemic compound, i.e., a mixture of at least two enantiomers in a ratio of substantially 50:50.

In each case, the compounds of formula (I) according to the invention are in free form, in oxidized form as a N-oxide or in salt form, e.g., an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

As used herein, the term “halogen” or “halo” refers to fluorine (fluoro), chlorine (chloro), bromine (bromo) or iodine (iodo), preferably fluorine, chlorine or bromine. This also applies, correspondingly, to halogen in combination with other meanings, such as haloalkyl, haloalkenyl, haloalkynyl, haloalkoxy, and halocycloalkyl.

As used herein, amino means a —NH₂ group.

As used herein, cyano means a —CN group.

As used herein, the term “hydroxyl” or “hydroxy” means an —OH group.

As used herein, the term “carboxylic acid” means a —COOH group.

As used herein, the term “C₁-Cₙ-alkyl” refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

As used herein, the term “C₃-Cₙ-cycloalkyl” refers to three (3) to n membered cycloalkyl radical such as cyclopropyl, cyclobutyl, cyclopentyl and cyclohexyl.

As used herein, the term “C₁-Cₙ-alkoxy” refers to a straight-chain or branched saturated alkyl radical having one (1) to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy and 1,1-dimethylethoxy. The term “C₂-Cₙ-alkenyloxy” as used herein refers to a straight-chain or branched alkenyl chain having two (2) to n carbon atoms (as mentioned above) which is attached via an oxygen atom.

As used herein, the term “C₁-Cₙ-alkoxy-C₁-Cₙ-alkyl” refers to an alkyl radical (as mentioned above) substituted with a C₁-Cₙ-alkoxy group. Examples are methoxymethyl, methoxyethyl, ethoxymethyl and propoxymethyl.

As used herein, the term “C₁-Cₙ-haloalkyl” refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. Accordingly, a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl. Similarly, the term “C₂-Cₙ-haloalkenyl” or “C₂-Cₙ-haloalkynyl” as used herein refers to a C₂-Cₙ-alkenyl or C₂-Cₙ-alkynyl radical respectively substituted with one or more halogen atoms which may be the same or different. Similarly, the term “C₃-Cₙ-halocycloalkyl” or “C₁-Cₙ-haloalkoxy” as used herein refers to a C₃-Cₙ-cycloalkyl radical or C₁-Cₙ-alkoxyl radical respectively substituted with one or more halo atoms which may be the same or different.

As used herein, the term “C₁-Cₙ-alkylcarbonyl” refers to a C₁-Cₙ-alkyl group linked through the carbon atom of a carbonyl (C═O) group.

As used herein, the term “C₁-Cₙ-alkoxycarbonyl” refers to a C₁-Cₙ-alkoxy moiety linked through a carbon atom of a carbonyl (or C═O) group.

As used herein, the term “C₁-Cₙ-alkoxycarbonyl-C₁-Cₙ-alkyl” refers to a C₁-Cₙ-alkyl radical substituted by a “C₁-Cₙ-alkoxycarbonyl group.

As used herein, the term “N—C₁-C₄ alkoxy-C—C₁-C₄ alkyl-carbonimidoyl” refers to a radical of the formula —C(Rₐ)═NO(R_{b}) where Rₐ is a C₁-C₄-alkyl radical as generally defined above, and R_{b} is a C₁-C₄-alkyl radical as generally defined above.

As used herein the term “N-hydroxy-C—C₁-C₄-alkyl-carbonimidoyl” refers to a radical of the formula —C(Rₐ)═NOH where Rₐ is a C₁-C₄-alkyl radical as generally defined above.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, using known techniques and by observing results obtained under analogous circumstances. In determining the effective amount, a number of factors are considered including, but not limited to the type of plant or derived product to be applied; the pest to be controlled and its lifecycle; the particular compound applied; the type of application; and other relevant circumstances.

As used herein, the term “room temperature” or “RT” or “rt” or “ambient temperature” refer to a temperature of about 15° C. to about 35° C. For example, rt can refer to a temperature of about 20° C. to about 30° C.

The following list provides definitions, including preferred definitions, for substituents R¹, R², R³, R⁴, R⁵, R⁶, R¹, R⁸, R⁹, R¹⁰, R¹¹, R¹², B¹, B², B³, B⁴, A¹, A², A³ and Z¹ with reference to the compounds of formula (I) of the present invention. For any one of these substituents, any of the definitions given below may be combined with any definition of any other substituent given below or elsewhere in this document.

In one embodiment of the invention R¹ is selected from C₁-C₄-alkyl. Preferably, R¹ is methyl, ethyl or isopropyl.

More preferably, R¹ is methyl.

In one embodiment of the invention, R² is selected from hydrogen, halogen, or C₁-C₄ alkyl. Preferably R² is hydrogen, fluorine, chlorine, bromine, methyl, or ethyl. More preferably R² is hydrogen, chlorine or methyl. In one preferred embodiment R² is hydrogen. In another preferred embodiment R² is methyl. In still another preferred embodiment R² is chlorine.

In one embodiment R³ is selected from hydrogen, or C₁-C₄ alkyl. Preferably R³ is hydrogen or methyl. More preferably R³ is hydrogen.

In one embodiment of the invention R⁴ is C₁-C₄-alkyl. Preferably R⁴ is methyl, or ethyl. More preferably R⁴ is hydrogen or methyl. In one embodiment of the invention R⁴ is hydrogen. In another embodiment of the invention R⁴ is methyl.

In one embodiment of the invention R⁵ and R⁶ are independently selected from hydrogen or C₁-C₄-alkyl.

Preferably R⁵ and R⁶ are hydrogen.

In one embodiment of the invention, A¹, A² and A³ are independently selected from CR⁷, N, NR⁸, O, or S, with the proviso that at least one of A¹, A² and A³ is selected from N, O or S, and that no more than one of A¹, A² and A³ is O or S. Preferably A¹, A² and A³ are independently selected from CR⁷, N, O, or S, with the proviso that at least one of A¹, A² and A³ is selected from N, O or S, and that no more than one of A¹, A² and A³ is O or S.

In one embodiment R⁷ is selected from hydrogen, or C₁-C₄-alkyl. Preferably R⁷ is hydrogen, methyl, or ethyl. More preferably R⁷ is hydrogen.

In one embodiment R⁸ is selected from hydrogen, or C₁-C₄-alkyl. Preferably R^{a} is hydrogen, methyl, or ethyl. More preferably R⁸ is hydrogen.

In one embodiment of the invention R⁹, R¹⁰, R¹¹ and R¹² are independently selected from hydrogen, halogen, cyano, C₁-C₄-alkyl, C₁-C₄-alkoxy, C₁-C₄-haloalkyl, C₁-C₄-haloalkoxy, C₁-C₄ alkoxy-C₁-C₄ alkyl, C₁-C₄ alkoxycarbonyl, C₁-C₄-alkylcarbonyl, N—C₁-C₄-alkoxy-C₁-C₄-alkyl-carbonimidoyl, N-hydroxy-C₁-C₄-alkyl-carbonimidoyl, or C₃-C₆-cycloalkyl, wherein said C₃-C₆ cycloalkyl is unsubstituted or substituted by 1 or 2 substituents independently selected from halogen, cyano, C₁-C₄-alkyl, C₁-C₄-haloalkyl, or C₁-C₄-alkoxy. Preferably R⁹, R¹⁰, R¹¹ and R¹² are independently selected from hydrogen, halogen, cyano, C₁-C₄-alkyl, C₁-C₄ alkoxy, C₁-C₄-alkylcarbonyl, or N—C₁-C₄-alkoxy-C₁-C₄-alkyl-carbonimidoyl. More preferably R⁹, R¹⁰, R¹¹ and R¹² are independently selected from hydrogen, chlorine, cyano, methyl, ethyl, methoxy, ethoxy, C₁-C₃ alkylcarbonyl, or N—C₁-C₂alkoxy-C₁-C₂-alkyl-carbonimidoyl. Even more preferably R⁹, R¹⁰, R¹¹ and R¹² are independently selected from hydrogen, chlorine, cyano, methyl, methoxy, methylcarbonyl, or N-methoxy-C-methyl-carbonimidoyl.

In one embodiment of the invention, B¹ is CR⁹ or N, B² is CR¹⁰ or N, B³ is CR¹¹ or N, B⁴ is CR¹² or N, with the proviso that only one of B¹, B², B³, and B⁴ is N. Preferably B¹ is CR⁹, B² is CR¹⁰, B³ is CR¹¹, and B⁴ is CR¹².

In one embodiment of the invention, Z¹ is selected from C₁-C₄-alkyl, phenyl, pyridyl, or C₃-C₆-cycloalkyl, wherein any of said phenyl, pyridyl and C₃-C₆ cycloalkyl are unsubstituted or substituted by 1 or 2 substituents independently selected from halogen, cyano, C₁-C₄-alkyl, C₁-C₄-haloalkyl, C₁-C₄-alkoxy, or C₁-C₄-haloalkoxy.

Preferably Z¹ is selected from phenyl, or pyridyl, wherein any of said phenyl and pyridyl are unsubstituted or substituted by 1 or 2 substituents independently selected from halogen, cyano, C₁-C₄-alkyl, C₁-C₄-haloalkyl, C₁-C₄ alkoxy, or C₁-C₄ haloalkoxy. Even more preferably Z¹ is selected from phenyl, or pyridyl, wherein any of said phenyl and pyridyl are unsubstituted or substituted by 1 or 2 substituents selected from halogen.

In one embodiment of the invention, Z¹ is selected from phenyl, 2,3,4-trifluorophenyl, 2,3-difluorophenyl, 3,4-difluorophenyl, 2,4,6-trifluorophenyl, 2,4-difluorophenyl, 2,5-difluorophenyl, 2-fluorophenyl, 3-fluorophenyl, 4-fluorophenyl, 2-chlorophenyl, 3-chlorophenyl, 4-chlorophenyl, 2,4-dichlorophenyl, 3,6-difluoro-2-pyridyl, 4,6-difluoro-2-pyridyl, 4,5-difluoro-2-pyridyl, 5,6-difluoro-2-pyridyl, 3-fluoro-4-pyridyl, 2-fluoro-4-pyridyl, 2,3-difluoro-4-pyridyl, 2,5-difluoro-4-pyridyl, 2,6-difluoro-4-pyridyl, 3,5-difluoro-4-pyridyl, 2,5-difluoro-4-pyridyl, 2-fluoro-3-pyridyl, 6-fluoro-3-pyridyl, 5-fluoro-3-pyridyl, 4-fluoro-3-pyridyl, 2,6-difluoro-3-pyridyl, 2,5-difluoro-3-pyridyl, 2,4-difluoro-3-pyridyl, 4,6-difluoro-3-pyridyl, 5,6-difluoro-3-pyridyl, 3,5-difluoro-2-pyridyl, 3,4-difluoro-2-pyridyl, 6-fluoro-2-pyridyl, 4-fluoro-2-pyridyl, 5-fluoro-2-pyridyl, 3-fluoro-2-pyridyl or 4,5-difluoro-3-pyridyl. Preferably Z¹ is selected from phenyl, 2,4-difluorophenyl, 2,5-difluorophenyl, 2-chlorophenyl, 2-fluorophenyl, 3,4-difluorophenyl, 3-chlorophenyl, 3-fluorophenyl, 4-fluorophenyl, 2,4-dichlorophenyl, 3-fluoro-2-pyridyl, 5-fluoro-2-pyridyl, 6-fluoro-2-pyridyl, 3,4-difluoro-2-pyridyl, 3,5-difluoro-2-pyridyl, 2-fluoro-4-pyridyl, or 2,6-difluoro-3-pyridyl. More preferably Z¹ is selected from phenyl, 2,4-difluorophenyl, 2,4-dichlorophenyl, 3,5-difluoro-2-pyridyl, 2-fluorophenyl, 4-fluorophenyl or 2,6-difluoro-3-pyridyl. Most preferably Z¹ is selected from phenyl, 2,4-difluorophenyl, 3,5-difluoro-2-pyridyl, 2,4-dichlorophenyl, or 2,6-difluoro-3-pyridyl.

The present invention, accordingly, makes available a compound of formula (I) having R¹, R², R³, R⁴, R⁵, R⁶, R¹, R⁸, R⁹, R¹⁰, R¹¹, R¹², B¹, B², B³, B⁴, A¹, A², A³ and Z¹ as defined above in all combinations/each permutation. The term “compound of formula (I)” refers to component A.

Embodiments according to the invention are provided as set out below.

In one embodiment of the invention, there is provided a fungicidal composition comprising a mixture of components (A) and (B) as active ingredients, wherein component (A) is a compound of formula (I):

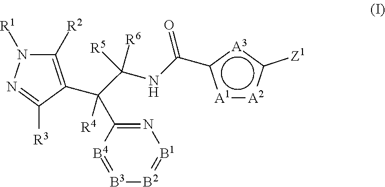

wherein R¹ is methyl; R² is hydrogen; R³ is hydrogen; R⁴ is methyl; R⁵ and R⁶ are hydrogen; A¹, A² and A³ are independently selected from CH, N, O, or S, with the proviso that at least one of A¹, A² and A³ is selected from N, O or S, and that no more than one of A¹, A² and A³ is O or S; B¹ is CR⁹, B² is CR¹⁰, B³ is CR¹¹, and B⁴ is CR¹²; R⁹, R¹⁰, R¹¹ and R¹² are independently selected from hydrogen, chlorine, cyano, methyl, methoxy, methylcarbonyl, or N-methoxy-C-methyl-carbonimidoyl; and Z¹ is selected from phenyl, or pyridyl, wherein any of said phenyl and pyridyl are unsubstituted or substituted by 1 or 2 substituents selected from halogen; or salt or N-oxide thereof,
and
component (B) is a compound selected from the group consisting of:
pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidine, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

In another embodiment of the invention, there is provided a fungicidal composition comprising a mixture of components (A) and (B) as active ingredients, wherein component (A) is a compound of formula (I):

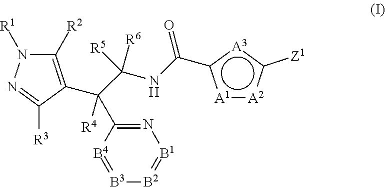

wherein R¹ is methyl; R² is hydrogen; R³ is hydrogen; R⁴ is methyl; R⁵ and R⁶ are hydrogen; A¹, A² and A³ are independently selected from CH, N, O, or S, with the proviso that at least one of A¹, A² and A³ is selected from N, O or S, and that no more than one of A¹, A² and A³ is 0 or S; B¹ is CR⁹, B² is CR¹⁰, B³ is CR¹¹, and B⁴ is CR¹²; R⁹, R¹⁰, R¹¹ and R¹² are independently selected from hydrogen, chlorine, cyano, methyl, methoxy, methylcarbonyl, or N-methoxy-C-methyl-carbonimidoyl; and Z¹ is selected from phenyl, or pyridyl, wherein any of said phenyl and pyridyl are unsubstituted or substituted by 1 or 2 substituents selected from halogen; or salt or N-oxide thereof, and
component (B) is a compound selected from the group consisting of:
pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A.

In still another embodiment of the invention, there is provided a fungicidal composition comprising a mixture of components (A) and (B) as active ingredients, wherein component (A) is a compound of formula (I):

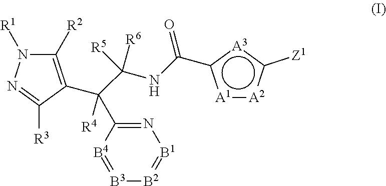

wherein R¹ is methyl; R² is hydrogen; R³ is hydrogen; R⁴ is methyl; R⁵ and R⁶ are hydrogen; A¹, A² and A³ are independently selected from CH, N, O, or S, with the proviso that at least one of A¹, A² and A³ is selected from N, O or S, and that no more than one of A¹, A² and A³ is O or S; B¹ is CR⁹, B² is CR¹⁰, B³ is CR¹¹, and B⁴ is CR¹²; R⁹, R¹⁰, R¹¹ and R¹² are independently selected from hydrogen, chlorine, cyano, methyl, methoxy, methylcarbonyl, or N-methoxy-C-methyl-carbonimidoyl; and Z¹ is selected from phenyl, or pyridyl, wherein any of said phenyl and pyridyl are unsubstituted or substituted by 1 or 2 substituents selected from halogen; or salt or N-oxide thereof, and
component (B) is a compound selected from the group consisting of:
pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), orAureobasidin A.

In a further embodiment of the invention, there is provided a fungicidal composition comprising a mixture of components (A) and (B) as active ingredients, wherein component (A) is a compound of formula (I):

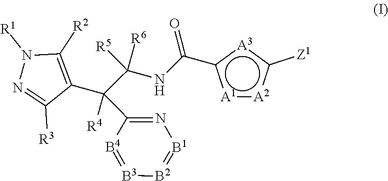

wherein R¹ is methyl; R² is hydrogen; R³ is hydrogen; R⁴ is methyl; R⁵ and R⁶ are hydrogen; A¹, A² and A³ are independently selected from CH, N, O, or S, with the proviso that at least one of A¹, A² and A³ is selected from N, O or S, and that no more than one of A¹, A² and A³ is O or S; B¹ is CR⁹, B² is CR¹⁰, B³ is CR¹¹, and B⁴ is CR¹²; R⁹, R¹⁰, R¹¹ and R¹² are independently selected from hydrogen, chlorine, cyano, methyl, methoxy, methylcarbonyl, or N-methoxy-C-methyl-carbonimidoyl; and Z¹ is selected from phenyl, or pyridyl, wherein any of said phenyl and pyridyl are unsubstituted or substituted by 1 or 2 substituents selected from halogen; or salt or N-oxide thereof, and
component (B) is a compound selected from the group consisting of:
pydiflumetofen, benzovindiflupyr, azoxystrobin, florylpicoxamid, difenoconazole, prothioconazole, mefentrifluconazole, cyprodinil, fludioxonil, and acibenzolar-S-methyl.

Preferably, component (A) is a compound selected from N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-(1-methylpyrazol-4-yl)-2-(2-pyridyl)propyl]isoxazole-3-carboxamide (X.03), N-[(2S)-2-(6-chloro-4-methoxy-2-pyridyl)-2-(1-methylpyrazol-4-yl)propyl]-3-(2,4-difluorophenyl)isoxazole-5-carboxamide (X.04), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), N-[2-(5-chloro-2-pyridyl)-2-(1-methylpyrazol-4-yl)propyl]-5-(2,4-difluorophenyl)-1,3,4-thiadiazole-2-carboxamide (X.06), N-[(2R)-2-(6-chloro-4-methoxy-2-pyridyl)-2-(1-methylpyrazol-4-yl)propyl]-3-(2,4-difluorophenyl)isoxazole-5-carboxamide (X.07), 5-(2,4-difluorophenyl)-N-[2-(1-methylpyrazol-4-yl)-2-(5-methyl-2-pyridyl)propyl]isoxazole-3-carboxamide (X.08), N-[2-(6-chloro-2-pyridyl)-2-(1-methylpyrazol-4-yl)propyl]-5-(2,4-dichlorophenyl)isoxazole-3-carboxamide (X.09), methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or one of the (S)- or (R)-enantiomers thereof, as defined in Table X below.

More preferably, component (A) is a compound selected from N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-(1-methylpyrazol-4-yl)-2-(2-pyridyl)propyl]isoxazole-3-carboxamide (X.03), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), N-[2-(5-chloro-2-pyridyl)-2-(1-methylpyrazol-4-yl)propyl]-5-(2,4-difluorophenyl)-1,3,4-thiadiazole-2-carboxamide (X.06), N-[(2R)-2-(6-chloro-4-methoxy-2-pyridyl)-2-(1-methylpyrazol-4-yl)propyl]-3-(2,4-difluorophenyl)isoxazole-5-carboxamide (X.07), methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or one of the (S)- or (R)-enantiomers thereof, as defined in Table X below.

Even more preferably, component (A) is a compound selected from: N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), N-[2-(5-chloro-2-pyridyl)-2-(1-methylpyrazol-4-yl)propyl]-5-(2,4-difluorophenyl)-1,3,4-thiadiazole-2-carboxamide (X.06), N-[(2R)-2-(6-chloro-4-methoxy-2-pyridyl)-2-(1-methylpyrazol-4-yl)propyl]-3-(2,4-difluorophenyl)isoxazole-5-carboxamide (X.07), N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or one of the (S)- or (R)-enantiomers thereof, as defined in Table X below.

TABLE X
Component (A)

| Entry | IUPAC name | Structure |
|---|---|---|
| X.01 | N-[2-(6-chloro-2-pyridyl)-2-(1- methylpyrazol-4-yl)propyl]-5-(2,4- difluorophenyl)isoxazole-3- carboxamide | 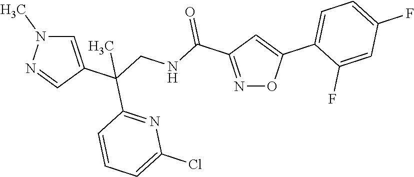 [SMILES: Cn1cc(C(C)(CNC(=O)c2cc(-c3ccc(F)cc3F)on2)c2cccc(Cl)n2)cn1] |
| X.02 | N-[2-(6-cyano-2-pyridyl)-2-(1- methylpyrazol-4-yl)propyl]-5-(2,4- difluorophenyl)isoxazole-3- carboxamide | 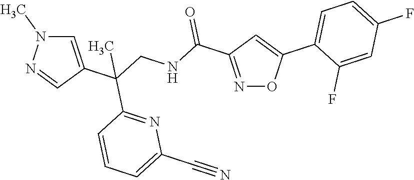 [SMILES: Cn1cc(C(C)(CNC(=O)c2cc(-c3ccc(F)cc3F)on2)c2cccc(C#N)n2)cn1] |
| X.03 | 5-(2,4-difluorophenyl)-N-[2-(1- methylpyrazol-4-yl)-2-(2- pyridyl)propyl]isoxazole-3- carboxamide | 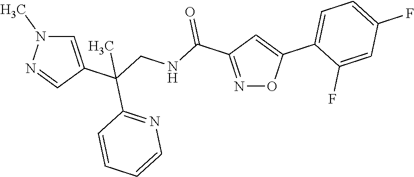 [SMILES: Cn1cc(C(C)(CNC(=O)c2cc(-c3ccc(F)cc3F)on2)c2ccccn2)cn1] |
| X.04 | N-[(2S)-2-(6-chloro-4-methoxy-2- pyridyl)-2-(1-methylpyrazol-4- yl)propyl]-3-(2,4- difluorophenyl)isoxazole-5- carboxamide | 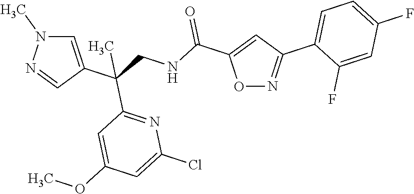 [SMILES: COc1cc(Cl)nc([C@](C)(CNC(=O)c2cc(-c3ccc(F)cc3F)no2)c2cnn(C)c2)c1] |
| X.05 | 5-(2,4-difluorophenyl)-N-[2-[6-[(E)- N-methoxy-C-methyl- carbonimidoyl]-2-pyridyl]-2-(1- methylpyrazol-4- yl)propyl]isoxazole-3-carboxamide | 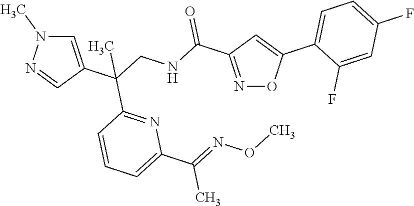 [SMILES: CO/N=C(\C)c1cccc(C(C)(CNC(=O)c2cc(-c3ccc(F)cc3F)on2)c2cnn(C)c2)n1] |
| X.06 | N-[2-(5-chloro-2-pyridyl)-2-(1- methylpyrazol-4-yl)propyl]-5-(2,4- difluorophenyl)-1,3,4-thiadiazole-2- carboxamide | 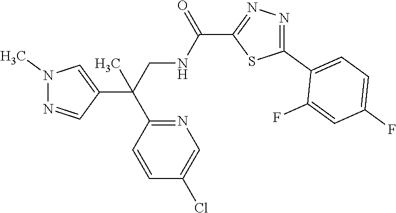 [SMILES: Cn1cc(C(C)(CNC(=O)c2nnc(-c3ccc(F)cc3F)s2)c2ccc(Cl)cn2)cn1] |
| X.07 | N-[(2R)-2-(6-chloro-4-methoxy-2- pyridyl)-2-(1-methylpyrazol-4- yl)propyl]-3-(2,4- difluorophenyl)isoxazole-5- carboxamide | 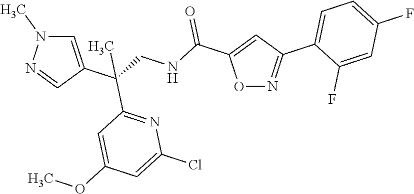 [SMILES: COc1cc(Cl)nc([C@@](C)(CNC(=O)c2cc(-c3ccc(F)cc3F)no2)c2cnn(C)c2)c1] |
| X.08 | 5-(2,4-difluorophenyl)-N-[2-(1- methylpyrazol-4-yl)-2-(5-methyl-2- pyridyl)propyl]isoxazole-3- carboxamide | 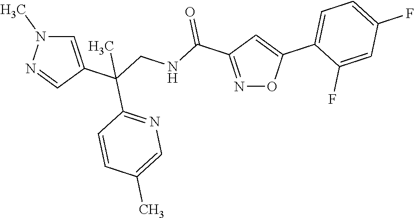 [SMILES: Cc1ccc(C(C)(CNC(=O)c2cc(-c3ccc(F)cc3F)on2)c2cnn(C)c2)nc1] |
| X.09 | N-[2-(6-chloro-2-pyridyl)-2-(1- methylpyrazol-4-yl)propyl]-5-(2,4- dichlorophenyl)isoxazole-3- carboxamide | 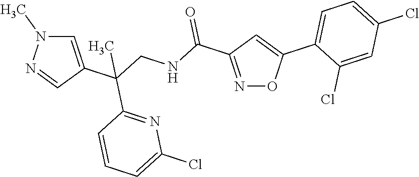 [SMILES: Cn1cc(C(C)(CNC(=O)c2cc(-c3ccc(Cl)cc3Cl)on2)c2cccc(Cl)n2)cn1] |
| X.10 | methyl 6-[2-[[5-(2,4- difluorophenyl)isoxazole-3- carbonyl]amino]-1-methyl-1-(1- methylpyrazol-4-yl)ethyl]pyridine-3- carboxylate | 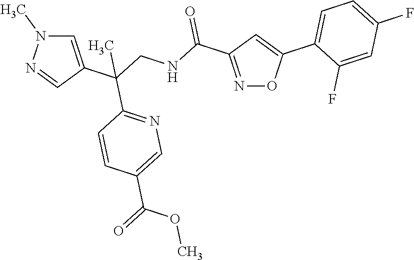 [SMILES: COC(=O)c1ccc(C(C)(CNC(=O)c2cc(-c3ccc(F)cc3F)on2)c2cnn(C)c2)nc1] |
| X.11 | N-[2-(6-chloro-2-pyridyl)-2-(1- methylpyrazol-4-yl)propyl]-5-(3,5- difluoro-2-pyridyl)isoxazole-3- carboxamide | 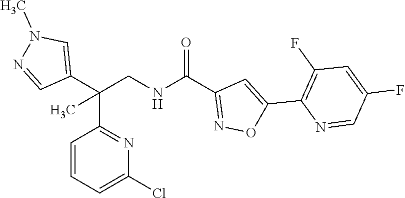 [SMILES: Cn1cc(C(C)(CNC(=O)c2cc(-c3ncc(F)cc3F)on2)c2cccc(Cl)n2)cn1] |
| X.12 | N-[2-(6-chloro-2-pyridyl)-2-(1- methylpyrazol-4-yl)propyl]-3-(2,4- difluorophenyl)-1,2,4-oxadiazole-5- carboxamide | 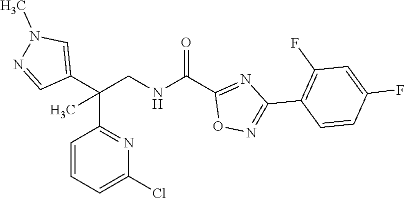 [SMILES: Cn1cc(C(C)(CNC(=O)c2nc(-c3ccc(F)cc3F)no2)c2cccc(Cl)n2)cn1] |
| X.13 | N-[2-(6-chloro-2-pyridyl)-2-(1- methylpyrazol-4-yl)propyl]-5-(2,4- difluorophenyl)-1,3,4-thiadiazole-2- carboxamide | 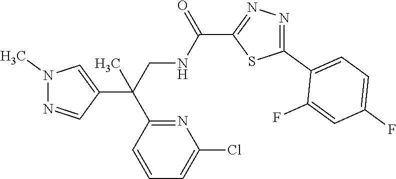 [SMILES: Cn1cc(C(C)(CNC(=O)c2nnc(-c3ccc(F)cc3F)s2)c2cccc(Cl)n2)cn1] |
| X.14 | N-[2-(6-cyano-2-pyridyl)-2-(1- methylpyrazol-4-yl)propyl]-5-(3,5- difluoro-2-pyridyl)-1,3,4- thiadiazole-2-carboxamide | 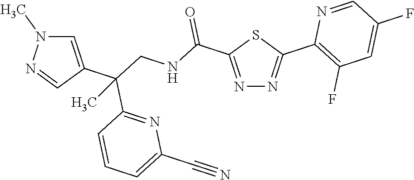 [SMILES: Cn1cc(C(C)(CNC(=O)c2nnc(-c3ncc(F)cc3F)s2)c2cccc(C#N)n2)cn1] |
| X.15 | N-[2-(6-chloro-2-pyridyl)-2-(1- methylpyrazol-4-yl)propyl]-5-(3,5- difluoro-2-pyridyl)-1,3,4- thiadiazole-2-carboxamide | 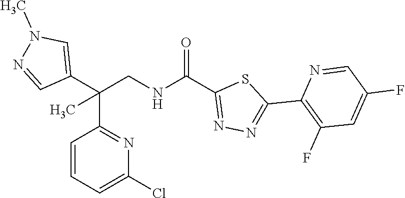 [SMILES: Cn1cc(C(C)(CNC(=O)c2nnc(-c3ncc(F)cc3F)s2)c2cccc(Cl)n2)cn1] |
| X.16 | N-[2-(6-chloro-2-pyridyl)-2-(1- methylpyrazol-4-yl)propyl]-5-(2,6- difluoro-3-pyridyl)-1,3,4- thiadiazole-2-carboxamide | 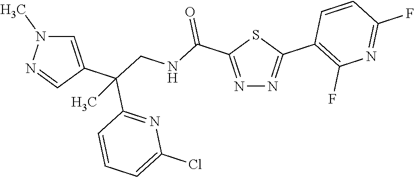 [SMILES: Cn1cc(C(C)(CNC(=O)c2nnc(-c3ccc(F)nc3F)s2)c2cccc(Cl)n2)cn1] |

In one embodiment component (B) is a compound selected from the group consisting of pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfiuxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidin, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-meth oxy-N-[[4-[5-(trifluoromethyl)-1,2,4-oxadiazol-3-yl]phenyl]meth yl]cyclopropane-carboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

Preferably, component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]-cyclopropanecarboxamide, N,2-dimethoxy-N-[[4-[5-(trifluoromethyl)-1,2,4-oxadiazol-3-yl]phenyl]methyl]-propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A.

More preferably, component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A.

Still more preferably, component (B) is a compound selected from pydiflumetofen, benzovindiflupyr, azoxystrobin, florylpicoxamid, difenoconazole, prothioconazole, mefentrifluconazole, cyprodinil, fludioxonil, or acibenzolar-S-methyl.

In one embodiment component (B) is a compound selected from pydiflumetofen, benzovindiflupyr, azoxystrobin, florylpicoxamid, difenoconazole, prothioconazole, mefentrifluconazole, cyprodinil, fludioxonil, or acibenzolar-S-methyl.

In another embodiment component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A.

In another prefered embodiment component (B) is a compound selected from pydiflumetofen, benzovindiflupyr, azoxystrobin, florylpicoxamid, difenoconazole, prothioconazole, mefentrifluconazole, cyprodinil, fludioxonil, or acibenzolar-S-methyl, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), or Aureobasidin A.

The component (B) compounds are referred to herein and above by a so-called “ISO common name” or another “common name” being used in individual cases or a trademark name. The component (B) compounds are known and are commercially available and/or can be prepared using procedures known in the art and/or procedures reported in the literature.

N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, these compounds may be prepared from the methods described in WO 2017/055473, WO 2017/055469, WO 2017/093348 and WO 2017/118689.

Methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, these compounds may be prepared from the methods described in WO2020/193387.

N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, these compounds may be prepared from the methods described in WO2017/153380. 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, these compounds may be prepared from the methods described in WO2017207362A1, WO2019105933A1, WO2020109509A1; 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, these compounds may be prepared from the methods described in WO2017207362A1, WO2019105933A1, WO2020109511 A1, WO2021244952A1. “TIMOREX Gold^{T}M” or “Timorex Gold®” as used herein, refers to Melaluca alternifolia oil, which is an extract of the tea tree plant Melaluca alternifolia, commercially available as Timorex Gold®, which is a broad-spectrum botanical biofungicide.

TAEGRO™ or TAEGRO® as used herein, refers to a microorganism-based fungicide formulated as a wettable powder containing 130 g/kg Bacillus amyloliquefaciens strain FZB24, having Accession No. DSM 10271 (13% w/w minimum of 1×1013 cfu/kg), commercially available as TAEGRO®.

BOTRISTOP® as used herein refers to a broad spectrum biofungicide, a plant extract based on the extract of Quillaja saponaria Molina.

“REGALIA®” as used herein, refers to a biofungicide, a plant extract based on the extract of Reynoutria sachalinensis extract (commercially available as REGALIA®).

Aureobasidin A is an antifungal cyclic depsipeptide antibiotic produced by Aureobasidium pullulans. See, for instance, Takesako et al., J. Antibiot. 1991, 44, 919-924. WO 2018/102345 discloses use of Aureobasidin A as an agricultural fungicide to treat, 10 prevent or control fungal infections in plants and seeds.

Enantiomerically pure final compounds may be obtained from racemic starting materials as appropriate via standard physical separation techniques, such as reverse phase chiral chromatography, or through stereoselective synthetic techniques, e.g., by using chiral starting materials.

The term “component A” refers to the compound of formula (I).

The following mixtures of components (A), wherein component (A) is a compound of formula(I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): pydiflumetofen+AX, benzovindiflupyr+AX, bixafen+AX, fluxapyroxad+AX, isopyrazam+AX, penflufen+AX, penthiopyrad+AX, sedaxane+AX, boscalid+AX, fluopyram+AX, thifluzamide+AX, pyraziflumid+AX, isoflucypram+AX, inpyrfluxam+AX, isofetamid+AX, fluindapyr+AX, cyclobutrifluram+AX, fluoxastrobin+AX, fenamidone+AX, mandestrobin+AX, picoxystrobin+AX, pyraclostrobin+AX, famoxadone+AX, kresoxim-methyl+AX, trifloxystrobin+AX, azoxystrobin+AX, metyltetraprole+AX, amisulbrom+AX, cyazofamid+AX, fenpicoxamid+AX, florylpicoxamid+AX, metarylpicoxamid+AX, ametoctradin+AX, fluazinam+AX, fentin hydroxide+AX, silthiofam+AX, fenpropimorph+AX, fenpropidin+AX, spiroxamine+AX, fenhexamid+AX, imazalil+AX, pyrisoxazole+AX, bromuconazole+AX, cyproconazole+AX, difenoconazole+AX, epoxiconazole, flutriafol+AX, hexaconazole+AX, ipconazole+AX, metconazole+AX, myclobutanil+AX, penconazole+AX, propiconazole+AX, tebuconazole+AX, tetraconazole+AX, triticonazole+AX, prothioconazole+AX, fluoxytioconazole+AX, mefentrifluconazole+AX, flufenoxadiazam+AX, ipflufenoquin+AX, quinofumelin+AX, metalaxyl-M+AX, cyprodinil+AX, pyrimethanil+AX, kasugamycin+AX, mancozeb+AX, copper fungicides+AX, sulphur+AX, zinc thiazole+AX, captan+AX, folpet+AX, chlorothalonil+AX, dithianon+AX, quinoxyfen+AX, proquinazid+AX, fludioxonil+AX, iprodione+AX, procymidone+AX, thiabendazole+AX, zoxamide+AX, metrafenone+AX, fluopicolide+AX, propamocarb+AX, oxathiapiprolin+AX, fluoxapiprolin+AX, acibenzolar-S-methyl+AX, isotianil+AX, phosphorous acid+AX, cyflufenamid+AX, tebufloquin+AX, picarbutrazox+AX, tricyclazole+AX, N-methoxy-N-[[4-[5-(trifluoromethyl)-1,2,4-oxadiazol-3-yl]phenyl]methyl]cyclopropanecarboxamide+AX, N,2-dimethoxy-N-[[4-[5-(trifluoromethyl)-1,2,4-oxadiazol-3-yl]phenyl]methyl]propanamide+AX, 1-methoxy-3-methyl-1-[[4-[5-(trifluoromethyl)-1,2,4-oxadiazol-3-yl]phenyl]methyl]urea+AX, 1,3-dimethoxy-1-[[4-[5-(trifluoromethyl)-1,2,4-oxadiazol-3-yl]phenyl]methyl]urea+AX, N-[(1R)-1-benzyl-3-chloro-1-methyl-but-3-enyl]-8-fluoro-quinoline-3-carboxamide+AX, N-[(1S)-1-benzyl-3-chloro-1-methyl-but-3-enyl]-8-fluoro-quinoline-3-carboxamide+AX, N-[(1R)-1-benzyl-3,3,3-trifluoro-1-methyl-propyl]-8-fluoro-quinoline-3-carboxamide+AX, N-[(1S)-1-benzyl-3,3,3-trifluoro-1-methyl-propyl]-8-fluoro-quinoline-3-carboxamide+AX, N-[(1R)-1-benzyl-1,3-dimethyl-butyl]-7,8-difluoro-quinoline-3-carboxamide+AX, N-[(1S)-1-benzyl-1,3-dimethyl-butyl]-7,8-difluoro-quinoline-3-carboxamide+AX, 8-fluoro-N-[(1R)-1-[(3-fluorophenyl)methyl]-1.3-dimethyl-butyl]quinoline-3-carboxamide+AX, 8-fluoro-N-[(1S)-1-[(3-fluorophenyl)methyl]-1,3-dimethyl-butyl]quinoline-3-carboxamide+AX, N-[(1R)-1-benzyl-1,3-dimethyl-butyl]-8-fluoro-quinoline-3-carboxamide+AX, N-[(1S)-1-benzyl-1,3-dimethyl-butyl]-8-fluoro-quinoline-3-carboxamide+AX, N-((1R)-1-benzyl-3-chloro-1-methyl-but-3-enyl)-8-fluoro-quinoline-3-carboxamide+AX, N-((1S)-1-benzyl-3-chloro-1-methyl-but-3-enyl)-8-fluoro-quinoline-3-carboxamide+AX, methyl (Z)-3-methoxy-2-[2-methyl-5-[4-(trifluoromethyl)triazol-2-yl]phenoxy]prop-2-enoate+AX, methyl (Z)-3-methoxy-2-[2-methyl-5-(4-propyltriazol-2-yl)phenoxy]prop-2-enoate+AX, methyl (Z)-2-[5-(3-isopropylpyrazol-1-yl)-2-methyl-phenoxy]-3-methoxy-prop-2-enoate+AX, methyl (Z)-3-methoxy-2-[2-methyl-5-(3-propylpyrazol-1-yl)phenoxy]prop-2-enoate+AX, methyl (Z)-3-methoxy-2-[2-methyl-5-[3-(trifluoromethyl)pyrazol-1-yl]phenoxy]prop-2-enoate+AX, methyl (Z)-2-(5-cyclohexyl-2-methyl-phenoxy)-3-methoxy-prop-2-enoate+AX, methyl (Z)-2-(5-cyclopentyl-2-methyl-phenoxy)-3-methoxy-prop-2-enoate+AX, 2-[cyano-(2,6-difluoro-4-pyridyl)amino]-5-methyl-N-spiro[3.4]octan-3-yl-thiazole-4-carboxamide+AX, 2-[cyano-(2,6-difluoro-4-pyridyl)amino]-N-(2,2-dimethylcyclobutyl)-5-methyl-thiazole-4-carboxamide+AX, 2-[cyano-(2,6-difluoro-4-pyridyl)amino]-N-hexyl-5-methyl-thiazole-4-carboxamide+AX, 2-[(2,6-difluoro-4-pyridyl)-(2-methoxyacetyl)amino]-N-(2,2-dimethylcyclobutyl)-5-methyl-thiazole-4-carboxamide+AX, 2-[(2,6-difluoro-4-pyridyl)-(tetrahydropyran-4-carbonyl)amino]-N-(2,2-dimethylcyclobutyl)-5-methyl-thiazole-4-carboxamide+AX, 2-[(2,6-difluoro-4-pyridyl)-(oxetane-3-carbonyl)amino]-N-(2,2-dimethylcyclobutyl)-5-methyl-thiazole-4-carboxamide+AX, 2-[(2,6-difluoro-4-pyridyl)-(tetrahydrofuran-3-carbonyl)amino]-N-(2,2-dimethylcyclobutyl)-5-methyl-thiazole-4-carboxamide+AX, 2-[acetyl-(2,6-difluoro-4-pyridyl)amino]-N-(2,2-dimethylcyclobutyl)-5-methyl-thiazole-4-carboxamide+AX, 2-[(2,6-difluoro-4-pyridyl)-(2-methylpropanoyl)amino]-N-(2,2-dimethylcyclobutyl)-5-methyl-thiazole-4-carboxamide+AX, TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP@)+AX, and Aureobasidin A+AX.

In another embodiment of the invention the following mixtures of components (A), wherein component (A) is a compound of formula(I) with component (B) as active ingredients are preferred (wherein the term “AX” means one component (A) selected from selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): pydiflumetofen+AX, benzovindiflupyr+AX, bixafen+AX, fluxapyroxad+AX, isopyrazam+AX, penflufen+AX, penthiopyrad+AX, sedaxane+AX, boscalid+AX, fluopyram+AX, thifluzamide+AX, pyraziflumid+AX, isoflucypram+AX, inpyrfluxam+AX, isofetamid+AX, fluindapyr+AX, cyclobutrifluram+AX, fluoxastrobin+AX, fenamidone+AX, mandestrobin+AX, picoxystrobin+AX, pyraclostrobin+AX, famoxadone+AX, kresoxim-methyl+AX, trifloxystrobin+AX, azoxystrobin+AX, metyltetraprole+AX, amisulbrom+AX, cyazofamid+AX, fenpicoxamid+AX, florylpicoxamid+AX, metarylpicoxamid+AX, ametoctradin+AX, fluazinam+AX, fentin hydroxide+AX, silthiofam+AX, fenpropimorph+AX, fenpropidin+AX, spiroxamine+AX, fenhexamid+AX, imazalil+AX, pyrisoxazole+AX, bromuconazole+AX, cyproconazole+AX, difenoconazole+AX, epoxiconazole, flutriafol+AX, hexaconazole+AX, ipconazole+AX, metconazole+AX, myclobutanil+AX, penconazole+AX, propiconazole+AX, tebuconazole+AX, tetraconazole+AX, triticonazole+AX, prothioconazole+AX, fluoxytioconazole+AX, mefentrifluconazole+AX, flufenoxadiazam+AX, ipflufenoquin+AX, quinofumelin+AX, metalaxyl-M+AX, cyprodinil+AX, pyrimethanil+AX, kasugamycin+AX, mancozeb+AX, copper fungicides+AX, sulphur+AX, zinc thiazole+AX, captan+AX, folpet+AX, chlorothalonil+AX, dithianon+AX, quinoxyfen+AX, proquinazid+AX, fludioxonil+AX, iprodione+AX, procymidone+AX, thiabendazole+AX, zoxamide+AX, metrafenone+AX, fluopicolide+AX, propamocarb+AX, oxathiapiprolin+AX, fluoxapiprolin+AX, acibenzolar-S-methyl+AX, isotianil+AX, phosphorous acid+AX, cyflufenamid+AX, tebufloquin+AX, picarbutrazox+AX, tricyclazole+AX, N-methoxy-N-[[4-[5-(trifluoromethyl)-1,2,4-oxadiazol-3-yl]phenyl]methyl]cyclopropanecarboxamide+AX, N,2-dimethoxy-N-[[4-[5-(trifluoromethyl)-1,2,4-oxadiazol-3-yl]phenyl]methyl]propanamide+AX, 1-methoxy-3-methyl-1-[[4-[5-(trifluoromethyl)-1,2,4-oxadiazol-3-yl]phenyl]methyl]urea+AX, 1,3-dimethoxy-1-[[4-[5-(trifluoromethyl)-1,2,4-oxadiazol-3-yl]phenyl]methyl]urea+AX, N-[(1R)-1-benzyl-3-chloro-1-methyl-but-3-enyl]-8-fluoro-quinoline-3-carboxamide+AX, N-[(1S)-1-benzyl-3-chloro-1-methyl-but-3-enyl]-8-fluoro-quinoline-3-carboxamide+AX, N-[(1R)-1-benzyl-3,3,3-trifluoro-1-methyl-propyl]-8-fluoro-quinoline-3-carboxamide+AX, N-[(1S)-1-benzyl-3,3,3-trifluoro-1-methyl-propyl]-8-fluoro-quinoline-3-carboxamide+AX, N-[(1R)-1-benzyl-1,3-dimethyl-butyl]-7,8-difluoro-quinoline-3-carboxamide+AX, N-[(1S)-1-benzyl-1,3-dimethyl-butyl]-7,8-difluoro-quinoline-3-carboxamide+AX, 8-fluoro-N-[(1R)-1-[(3-fluorophenyl)methyl]-1,3-dimethyl-butyl]quinoline-3-carboxamide+AX, 8-fluoro-N-[(1S)-1-[(3-fluorophenyl)methyl]-1,3-dimethyl-butyl]quinoline-3-carboxamide+AX, N-[(1R)-1-benzyl-1,3-dimethyl-butyl]-8-fluoro-quinoline-3-carboxamide+AX, N-[(1S)-1-benzyl-1,3-dimethyl-butyl]-8-fluoro-quinoline-3-carboxamide+AX, N-((1R)-1-benzyl-3-chloro-1-methyl-but-3-enyl)-8-fluoro-quinoline-3-carboxamide+AX, N-((1S)-1-benzyl-3-chloro-1-methyl-but-3-enyl)-8-fluoro-quinoline-3-carboxamide+AX, methyl (Z)-3-methoxy-2-[2-methyl-5-[4-(trifluoromethyl)triazol-2-yl]phenoxy]prop-2-enoate+AX, methyl (Z)-3-methoxy-2-[2-methyl-5-(4-propyltriazol-2-yl)phenoxy]prop-2-enoate+AX, methyl (Z)-2-[5-(3-isopropylpyrazol-1-yl)-2-methyl-phenoxy]-3-methoxy-prop-2-enoate+AX, methyl (Z)-3-methoxy-2-[2-methyl-5-(3-propylpyrazol-1-yl)phenoxy]prop-2-enoate+AX, methyl (Z)-3-methoxy-2-[2-methyl-5-[3-(trifluoromethyl)pyrazol-1-yl]phenoxy]prop-2-enoate+AX, methyl (Z)-2-(5-cyclohexyl-2-methyl-phenoxy)-3-methoxy-prop-2-enoate+AX, methyl (Z)-2-(5-cyclopentyl-2-methyl-phenoxy)-3-methoxy-prop-2-enoate+AX, 2-[cyano-(2,6-difluoro-4-pyridyl)amino]-5-methyl-N-spiro[3.4]octan-3-yl-thiazole-4-carboxamide+AX, 2-[cyano-(2,6-difluoro-4-pyridyl)amino]-N-(2,2-dimethylcyclobutyl)-5-methyl-thiazole-4-carboxamide+AX, 2-[cyano-(2,6-difluoro-4-pyridyl)amino]-N-hexyl-5-methyl-thiazole-4-carboxamide+AX, 2-[(2,6-difluoro-4-pyridyl)-(2-methoxyacetyl)amino]-N-(2,2-dimethylcyclobutyl)-5-methyl-thiazole-4-carboxamide+AX, 2-[(2,6-difluoro-4-pyridyl)-(tetrahydropyran-4-carbonyl)amino]-N-(2,2-dimethylcyclobutyl)-5-methyl-thiazole-4-carboxamide+AX, 2-[(2,6-difluoro-4-pyridyl)-(oxetane-3-carbonyl)amino]-N-(2,2-dimethylcyclobutyl)-5-methyl-thiazole-4-carboxamide+AX, 2-[(2,6-difluoro-4-pyridyl)-(tetrahydrofuran-3-carbonyl)amino]-N-(2,2-dimethylcyclobutyl)-5-methyl-thiazole-4-carboxamide+AX, 2-[acetyl-(2,6-difluoro-4-pyridyl)amino]-N-(2,2-dimethylcyclobutyl)-5-methyl-thiazole-4-carboxamide+AX, 2-[(2,6-difluoro-4-pyridyl)-(2-methylpropanoyl)amino]-N-(2,2-dimethylcyclobutyl)-5-methyl-thiazole-4-carboxamide+AX, TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, and Aureobasidin A+AX, wherein the ratio of weight ratio of component (A) to component (B) is from 100:1 to 1:40, preferably from 40:1 to 1:40, even more preferably from 20:1 to 1:20, still more preferably from 15:1 to 1:30, still even more preferably from 10:1 to 1:10, most preferably from 5:1 to 1:5 and even most preferably from 2:1 to 1:2.

In another embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, or Aureobasidin A+AX.

In another embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, or Aureobasidin A+AX, wherein the weight ratio of component (A) to component (B) is from 100:1 to 1:40.

In another preferred embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, orAureobasidin A+AX, wherein the weight ratio of component (A) to component (B) is from 40:1 to 1:40.

In another more preferred embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, or Aureobasidin A+AX, wherein the weight ratio of component (A) to component (B) is from 40:1 to 1:20.

In another even more preferred embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, orAureobasidin A+AX, wherein the weight ratio of component (A) to component (B) is from 20:1 to 1:20.

In a still more preferred embodiment of the invention the following mixtures of components (A), wherein component (A) is a compound of formula (I) with component (B) as active ingredients are preferred (wherein the term “AX” means one component (A) selected from compounds of formula (I), or compounds selected from (X.01), (X.02), (X.03), (X.05), (X.06), (X.07), (X.10), (X.11), (X.12), (X.13), or (X.15), listed in table X according to the present invention): TAEGRO® (i.e, Bacillus amyloliquefaciens strain FZB24)+AX, Melaluca alternifolia oil (an extract of the tea tree plant Melaluca alternifolia (commercially available as Timorex Gold®, which is a broad-spectrum botanical biofungicide))+AX, Reynoutria sachalinensis extract (commercially available as REGALIA®)+AX, a plant extract based on the extract of Quillaja saponaria Molina (commercially available as BOTRISTOP®)+AX, or Aureobasidin A+AX, wherein the weight ratio of component (A) to component (B) is from 20:1 to 1:20.

In another still more preferred embodiment of the invention component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from TAEGRO® (i.e, Bacillus amyloliquefaciens strain FZB24), melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), orAureobasidin A, wherein the weight ratio of component (A) to component (B) is from 40:1 to 1:40, preferably from 20:1 to 1:20.

In another still more preferred embodiment of the invention component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In another still more preferred embodiment of the invention component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20. In another still more preferred embodiment of the invention component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 40:1 to 1:40, preferably from 20:1 to 1:20.

In a preferred composition according to the invention component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2.6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another most preferred composition according to the invention component (A) is compound no. X.14: N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethyl-cyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methyl propanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In a preferred composition according to the invention component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another most preferred composition according to the invention component (A) is compound no. X.14: N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 15:1 to 1:30.

In another preferred composition according to the invention component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or

In a preferred composition according to the invention component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from the group consisting pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP@), or Aureobasidin A, wherein the weight ratio of component (A) to component (B) is from 10:1 to 1:10 (or even more preferably, 5:1 to 1:5).

In another preferred composition according to the invention component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5) In another preferred composition according to the invention component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2.6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another most preferred composition according to the invention component (A) is compound no. X.14: N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In a preferred composition according to the invention component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A wherein the weight ratio of component (A) to component (B) is from 10:1 to 1:10 (or even more preferably, 5:1 to 1:5).

In another preferred composition according to the invention component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5). In another preferred composition according to the invention component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another most preferred composition according to the invention component (A) is compound no. X.14: N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A, wherein the weight ratio of component (A) to component (B) is from 10:1 to 1:10 (or even more preferably, 5:1 to 1:5).

In another preferred composition according to the invention component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), or Aureobasidin A wherein the weight ratio of component (A) to component (B) is from 10:1 to 1:10 (or even more preferably, 5:1 to 1:5).

In a preferred composition according to the invention, component (A) is compound no. X.01: N-[2-(6-chloro-2-pyridyl)-2-(1-methylpyrazol-4-yl)propyl]-5-(2,4-difluorophenyl)isoxazole-3-carboxamide (X.01), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.02: N-[2-(6-cyano-2-pyridyl)-2-(1-methylpyrazol-4-yl)propyl]-5-(2,4-difluorophenyl)isoxazole-3-carboxamide (X.02), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.03: 5-(2,4-difluorophenyl)-N-[2-(1-methylpyrazol-4-yl)-2-(2-pyridyl)propyl]isoxazole-3-carboxamide (X.03), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.04: N-[(2S)-2-(6-chloro-4-methoxy-2-pyridyl)-2-(1-methylpyrazol-4-yl)propyl]-3-(2,4-difluorophenyl)isoxazole-5-carboxamide (X.04), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.05: 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.06: N-[2-(5-chloro-2-pyridyl)-2-(1-methylpyrazol-4-yl)propyl]-5-(2,4-difluorophenyl)-1,3,4-thiadiazole-2-carboxamide (X.06), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.07: N-[(2R)-2-(6-chloro-4-methoxy-2-pyridyl)-2-(1-methylpyrazol-4-yl)propyl]-3-(2,4-difluorophenyl)isoxazole-5-carboxamide (X.07), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.08: 5-(2,4-difluorophenyl)-N-[2-(1-methylpyrazol-4-yl)-2-(5-methyl-2-pyridyl)propyl]isoxazole-3-carboxamide (X.08), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.09: N-[2-(6-chloro-2-pyridyl)-2-(1-methylpyrazol-4-yl)propyl]-5-(2,4-dichlorophenyl)isoxazole-3-carboxamide (X.09), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.10: methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.11: N-[2-(6-chloro-2-pyridyl)-2-(1-methylpyrazol-4-yl)propyl]-5-(3,5-difluoro-2-pyridyl)isoxazole-3-carboxamide (X.11), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.12: N-[2-(6-chloro-2-pyridyl)-2-(1-methylpyrazol-4-yl)propyl]-3-(2,4-difluorophenyl)-1,2,4-oxadiazole-5-carboxamide (X.12), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.13: N-[2-(6-chloro-2-pyridyl)-2-(1-methylpyrazol-4-yl)propyl]-5-(2,4-difluorophenyl)-1,3,4-thiadiazole-2-carboxamide (X.13), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another most preferred composition according to the invention, component (A) is compound no. X.14: N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.15: N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

In another preferred composition according to the invention, component (A) is compound no. X.16: N-[2-(6-chloro-2-pyridyl)-2-(1-methylpyrazol-4-yl)propyl]-5-(2,6-difluoro-3-pyridyl)-1,3,4-thiadiazole-2-carboxamide (X.16), or a salt enantiomer, tautomer or N-oxide thereof, and component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, or acibenzolar-S-methyl, wherein the weight ratio of component (A) to component (B) is from 10:1 to 1:10 (or even more preferably, 5:1 to 1:5).

The compounds of formula (I) according to the present invention can be made as shown in the following Schemes below, in which, unless otherwise stated, the definition of each variable is as defined above for a compound of formula (I).

The term “compounds of formula (I)” refers to component A.

In any of the compositions according to the invention, the composition may comprise an additional active ingredient component (C), which is different to component (B), and is selected from the group consisting of pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidine, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1.3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

Preferably, component (C), which is different to component (B), is a compound selected from the group consisting of pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

Even more preferably, component (C), which is different to component (B), is a compound selected from the group consisting of pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP@), and Aureobasidin A.

In another embodiment of the present invention, there is provided a fungicidal composition comprising a mixture of component (A) and a component (B) and a component (C) as active ingredients, wherein component (A) is a compound of formula (I) selected from compounds (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention, and wherein component (C), and component (B), are a compound selected from the group consisting of pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidine, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A, and wherein component (B) and (C) are not the same compound.

Preferably, there is provided a fungicidal composition comprising a mixture of component (A) and a component (B) and a component (C) as active ingredients, wherein component (A) is a compound of formula (I) selected from (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention, and wherein component (C), and component (B), are a compound selected from the group consisting of pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A, and wherein component (B) and (C) are not the same compound.

More preferably, there is provided a fungicidal composition comprising a mixture of component (A) and a component (B) and a component (C) as active ingredients, wherein component (A) is a compound of formula (I) selected from compounds (X.01), (X.02), (X.03), (X.04), (X.05), (X.06), (X.07), (X.08), (X.09), (X.10), (X.11), (X.12), (X.13), (X.14), (X.15), or (X.16), listed in table X according to the present invention, and wherein component (C), and component (B), are a compound selected from the group consisting of pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP@), and Aureobasidin A, and wherein component (B) and (C) are not the same compound.

The component (C) compounds are referred to herein and above by a so-called “ISO common name” or another “common name” being used in individual cases or a trademark name. The component (C) compounds are known and are commercially available and/or can be prepared using procedures known in the art and/or procedures reported in the literature such as indicated above.

Components (B) and (C) in combination with component (A) may enhance the effectiveness of the latter against fungi, and vice versa. Additionally, the fungicidal compositions may be effective against a wider spectrum of fungal pathogens that can be combated with the individual active ingredients when used solely.

Generally, the weight ratio of component (A) to the mixture of components (B) and (C) may be from 100:1 to 1:100, or 50:1 to 1:50, or 20:1 to 1:20, or 10:1 to 1:10, or 5:1 and 1:5. Otherwise, the weight ratio of component (A) to the mixture of components (B) and (C) may be from 2:1 to 1:2, or 4:1 to 2:1, or 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

In embodiments of the invention where the composition comprise a component (A), a component (B) and a component (C), the weight ratio of component (A) to the sum of component (B) and component (C) may be from 100:1 to 1:100, preferably from 50:1 to 1:50, more preferably from 20:1 to 1:40, even more preferably from 15:1 to 1:30, still more preferably from 12:1 to 1:25, or from 10:1 to 1:20, or from 10:1 to 1:10, or from 5:1 to 1:15, or from 5:1 to 1:5, or from 4:1 to 1:4, or from 3:1 to 1:10, or from 3:1 to 1:3, or from 2:1 to 1:5, or 1:1.

In any of the Schemes below, the presence of one or more possible asymmetric carbon atoms in a compound of formula (I) according to the invention means that the compounds may occur in chiral isomeric forms, i.e., enantiomeric or diastereomeric forms.

Compounds of formula (I) may be prepared from compounds of formula (III) by reaction with a compound of formula (II) using dicyclohexylcarbodiimide (DCC), diisopropylcarbodiimide (DIC) or N-(3-Dimethylaminopropyl)-N′-ethylcarbodiimide (EDAC·HCl) together with an additive such as 1-hydroxybenzotriazole (HOBt), Hydroxy-3,4-dihydro-4-oxo-1,2,3-benzotriazine (HODhbt), N-hydroxysuccinimide (HOSu), 1-Hydroxy-7-aza-1H-benzotriazole (HOAt) or 4-(N,N-dimethylamino)pyridine (DMAP). This reaction is shown in Scheme 1.

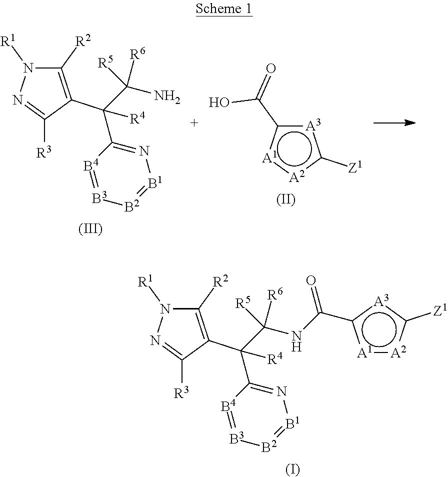

Alternatively, compounds of formula (I) may be prepared by reacting a compound of formula (IIa) with a compound of formula (III) in an inert solvent such as tetrahydrofuran (THF), ethyl acetate, methylene chloride, toluene and the like, optionally in the presence of an inorganic base, for example aqueous sodium hydroxide, or potassium carbonate, or in the presence of an organic base such as trimethylamine or diisopropyl amine. The latter reaction with organic bases can optionally be carried out in the presence of a catalyst such as 4-dimethylaminopyridine (DMAP). Compounds of formula (IIa), wherein X⁰ is halogen, preferably chlorine, can be prepared from compounds of formula (II) by treatment with a halogenating agent, such as thionyl chloride (SOCl₂) or oxalyl chloride (COCl₂), in an inert solvent as noted above, optionally mediated by the presence of catalytic quantities of N,N-dimethyl formamide (DMF). This reaction is shown in Scheme 2.

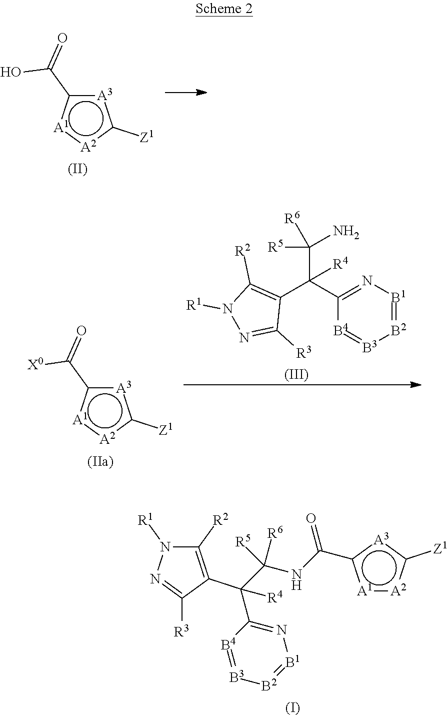

Acylation reactions of carboxylic acids such as compounds of formula (II) with amines such as compounds of formula (III) are well known to those skilled in the art and described for example in Eur. J. Org. Chem. 2020, 4641-4651, and references cited therein, and by methods described vide infra.

Compounds of formula (II) are commercially available or can be prepared according to, or analogous to, procedures described in the literature. For example, WO2018/019929, Org. Proc. Res. Dev. 2020, 24(2), 228-234, WO2018/019929, Lett. Org. Chem. 2010, 7(7), 502-507, and J. Het Chem. 2015, 52(6), 1823-1833.

Compounds of formula (III), or salts thereof, wherein R¹, R², R³, R⁴, R⁵, R⁶, B¹, B², B³ and B⁴ are as defined above for the compound of formula (I), may be prepared by a person skilled in the art by a reaction between nitriles of formula (IV), wherein R¹, R², R³, R⁴, B¹, B², B³ and B⁴ are as defined above for the compound of formula (I), and a suitable nucleophile such as (dimethyl sulfide)dihydroboron (BMS) in a suitable aprotic solvent such as tetrahydrofuran, for example as described in The J. Org. Chem. 1981 47, 3153. Alternatively, Grignard reagents R⁵MgBr or R⁶MgBr, wherein R⁵ and R⁶ are as defined above for the compound of formula (I), may be added as nucleophiles to compounds of formula (IV), sequentially or simultaneously, to allow more highly substituted amines of formula (III) to be prepared. Such Grignard additions to nitriles are carried out in an inert solvent such as diethyl ether, tert-butylmethyl ether, and cyclopentyl methyl ether in the presence of a Lewis acid such as Ti(O-ⁱPr)₄ (see Synlett 2007, (4), 652-654). This reaction is shown in Scheme 3.

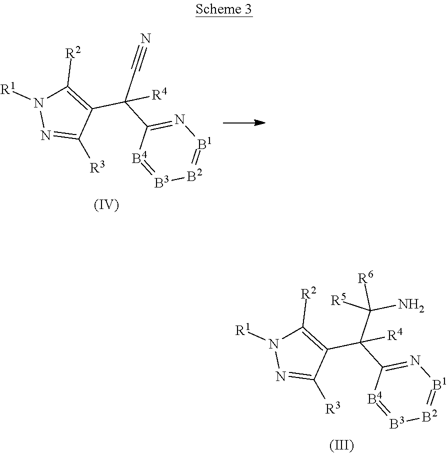

Compounds of formula (IV), wherein R¹, R², R³, R⁴, B¹, B², B³ and B⁴ are as defined above for the compound of formula (I), may be prepared by a person skilled in the art following known methods. More specifically, compounds of formula (IV), and intermediates thereof, may be prepared from compounds of formula (V) as shown in Scheme 4.

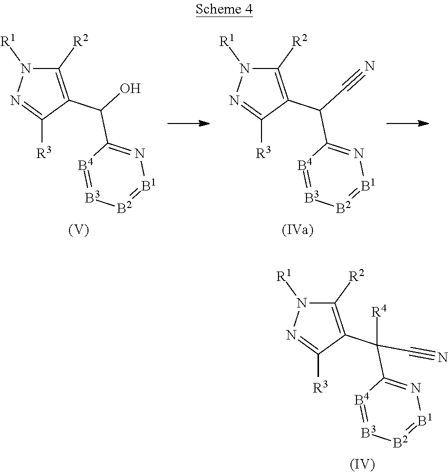

For example, compounds of formula (IV), wherein R¹, R², R³, R⁴, B¹, B², B³ and B⁴ are as defined above for the compound of formula (I) and R⁴ is different from hydrogen, may be prepared by a person skilled in the art by deprotonation of compound of formula (IVa), wherein R⁴ is hydrogen and R¹, R², R³, B¹, B², B³ and B⁴ are as defined above for the compound of formula (I), using a strong base such as n-butyl lithium or sodium hydride at cryogenic temperatures in an inert solvent such as tetrahydrofuran, followed by addition of a suitable alkylating agent R⁴—X⁰, wherein X⁰ is halogen, for example iodomethane. Compounds of formula (IVa), wherein R⁴ is hydrogen and R¹, R², R³, B¹, B², B³ and B⁴ are as defined above for the compound of formula (I), may be prepared from alcohols of formula (V) by treatment with cyanotrimethylsilane (TMSCN) in the presence of a base such as lithium carbonate in a nonpolar solvent such as dichloromethane at temperatures between 0° C. and the boiling point of the reaction mixture. Such transformations are well known in the literature under a variety of conditions, for example as described in Org. Lett. 2008, 10, 4570 and references therein. This reaction is shown in Scheme 4.

Compounds of formula (V) may be prepared from compounds of formula (VI), respectively from any of compounds of formula (VIa), (VIb), (VIc), (VId) or (VIe), as shown in Scheme 5.

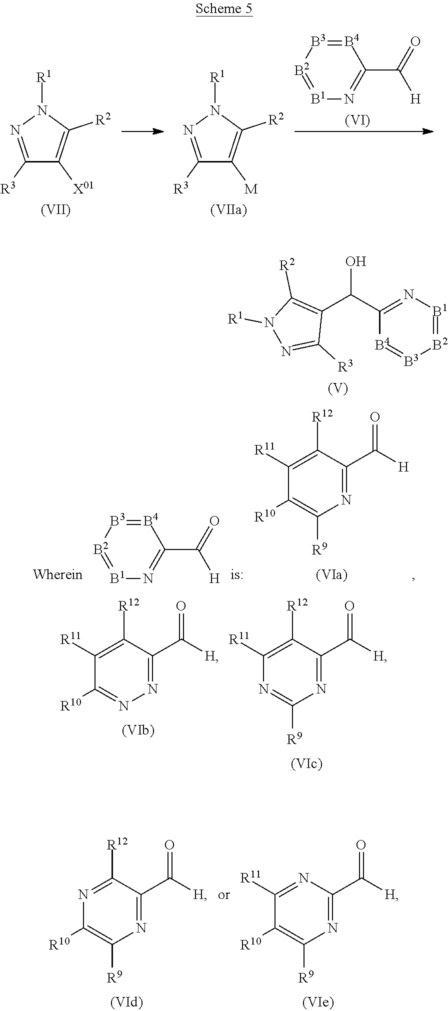

As shown in Scheme 5, compounds of formula (VII), wherein R¹, R² and R³ are as defined above for the compound of formula (I) and X⁰¹ is bromo or iodo, are metallated with an appropriate reagent such as turbo Grignard (isopropyl magnesium chloride-lithium chloride complex), or an alkyl lithium, such as n-butyl lithium to give an intermediate Grignard or alkyl lithium reagent (M is MgX⁰¹ or Lithium). Such metal insertions into C—X⁰¹ bonds are well known to those skilled in the art and are generally carried out at temperatures between −78° C. to room temperature, in inert solvents such as ethers, e.g., tert-butyl methyl ether or tetrahydrofuran and the like. Solutions of the metallated species (VIIa) are then treated with compounds of formula (VI), respectively (VIa), (VIb), (VIc), (VId), or (VIe) to give compounds of formula (V). Similar reactions of these type have been described in for example WO2012/102297 and Bio. Med. Chem. Lett. 2017, 27(17), 4044-4050 (X⁰¹ is Br, n-butyl lithium) and Ang. Chem. Int. Ed. 2016, 55(17), 5332-5336, US 2014/0349990, WO2002/004424, WO2021/009068 (X⁰¹ is I, Turbo Grignard).

Compounds of formula (VI) and (VII) are either commercially available or are readily prepared by methods known by those skilled in the art.

A further synthesis of compounds of formula (I) involves treatment of compounds of formula (VIII) with a base, such as sodium hydride or n-butyl lithium, in an inert solvent, such as tetrahydrofuran, and subsequent alkylation with compounds of formula (IX), wherein R⁴ is as described under formula (I) and X⁰² is a leaving group such as halogen, mesylate or tosylate, to yield compounds of formula (X) (Scheme 6).

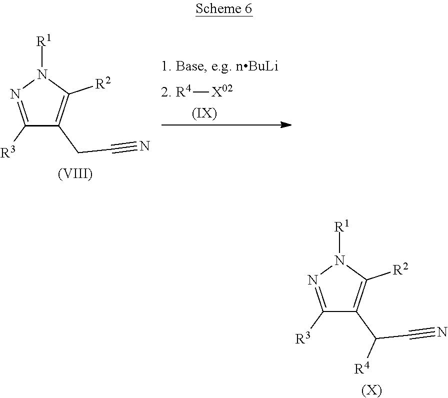

Compounds of formula (X), wherein R¹, R², R³, and R⁴ are as defined above for the compound of formula (I), are then treated with a strong base such as sodium hydride or an alkyl lithium base such as n-butyl lithium in an inert solvent, such as tetrahydrofuran or tert-butyl methyl ether, at temperatures between −78° C. to room temperature, followed by addition of a compound of formula (XI), respectively any of compounds of formula (XIa), (XIb), (XIc), (XId) or (XIe), wherein R⁹, R¹⁰, R¹¹, and R¹² are as defined above for the compound of formula (I), and X⁰³ is a leaving group such as halogen, preferably F, Cl or Br to give compounds of formula (IV). This reaction is shown in Scheme 7.

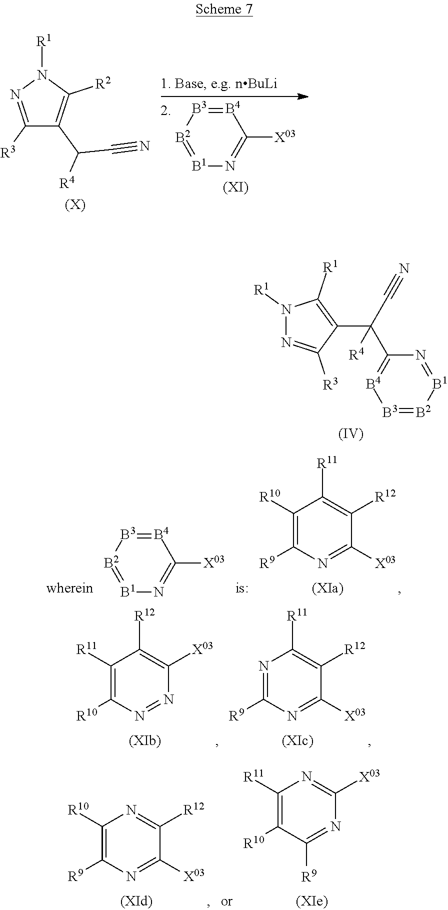

Compounds of formula (IV) are converted into compounds of formula (I) as previously described in Schemes 1, 2 and 3. Those skilled in the art will recognize that conversion of compounds (VIII) into compounds of formula (IV) can be carried out sequentially or in the same reaction vessel, enabling a streamlined conversion of compounds of formula (VIII) to compounds of formula (IV). This is described in more details in the preparative examples.

Compounds of formula (Ia), wherein R¹, R², R³, R⁵, B¹, B², B³, B⁴, A¹, A², A³ and Z¹ are as described above for compounds of formula (I) and R⁴ and R⁶ are hydrogen, can also be prepared by treatment of compounds of formula (VI), respectively any of compounds of formula (VIa), (VIb), (VIc), (VId) or (VIe), with compounds of formula (XII), wherein R⁵ is as described above for compounds of formula (I) in the presence of a base, such as triethyl amine, optionally in an inert solvent, such as ethanol or methanol, to give compounds of formula (XIII). These compounds may be isolated and converted to compounds of formula (XIV) by treatment with an anhydride, such as trifluoroacetic acid anhydride, in an inert solvent, such as methylene chloride, in the presence of a base, for example triethyl amine. This reaction is shown in Scheme 8.

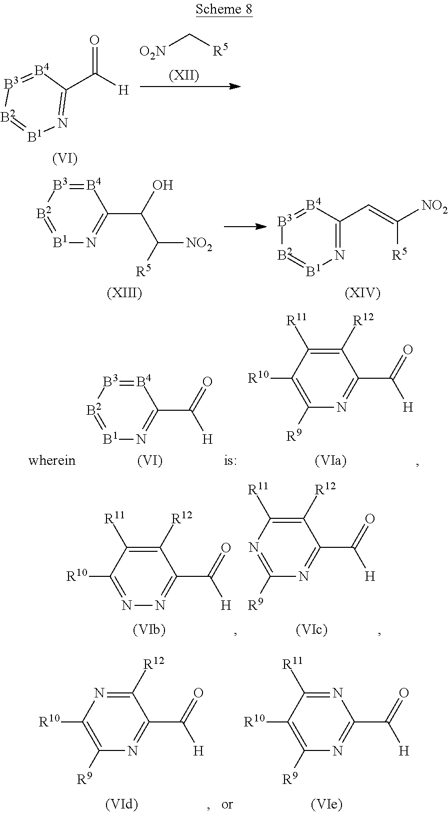

Those skilled in the art will appreciate that compounds of formula (VI) can be converted into compounds of formula (XIV) without isolation of the intermediates of formula (XIII). Such reactions, known as the Henry reactions, are well described in the literature, as evidenced in Tetrahedron 2001, 57(6), 915-945, and references cited therein. Compounds of formula (XIV) can be converted into compounds of formula (XV) by treatment with compounds of formula (VIIa) (see Scheme 5) in an inert solvent such as tetrahydrofuran. This reaction is shown in Scheme 9.

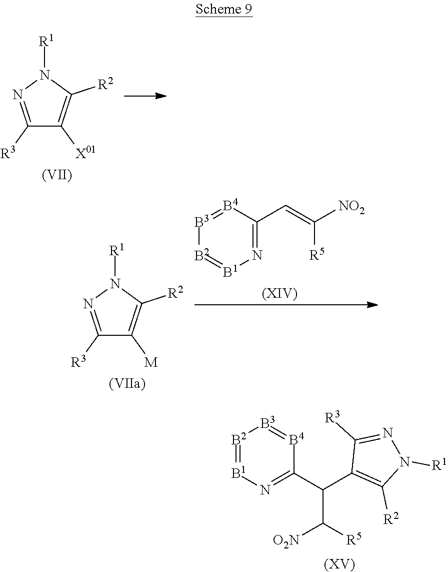

Similar Michael additions of organometallics to nitro alkenes have been reported for example in Org. Lett. 2007, 9, 85-87. Reduction of the nitro group in compounds of formula (XV) to the amine to give compounds of formula (IIIa), wherein R¹, R², R³, R⁵, B¹, B², B³ and B⁴ are as defined above for the compound of formula (I) and R⁴ and R⁶ are hydrogen, can be achieved by a multitude of methods generally known to those skilled in the art, such as Bechamp reduction, or reduction with hydrogen in the presence of a metal catalyst. This reaction is shown in Scheme 10.

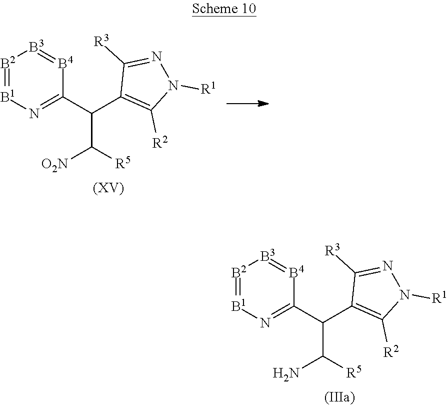

Compounds of formula (IIIa) are converted into compounds of formula (Ia) by the methods described in Schemes 1 and 2.

Further compounds according to the invention can be prepared by derivatization at a later stage in the synthesis using a key central intermediate. For example, compounds of formula (I), wherein B¹ is C—X⁰⁴, B² is CR¹⁰, B³ is CR¹¹, B⁴ is CR¹², R¹, R², R³, R⁴, R⁵, R⁶, R¹⁰, R¹¹, R¹², A¹, A², A³ and Z¹ are as defined above for the compounds of formula (I), and X⁰⁴ is halogen, preferably bromine or chlorine, i.e. compounds of formula (Ia):

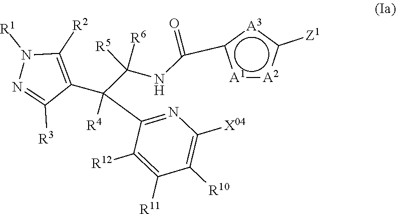

allow further chemistry to be carried out such as palladium catalysed carbonylations, Suzuki reactions, Stille couplings, copper catalysed introduction of sulfonyl groups, haloalkyl groups, and cyano moieties, as well as SnAr reactions with a variety of nucleophiles. Examples of such reactions are shown in Scheme 11.

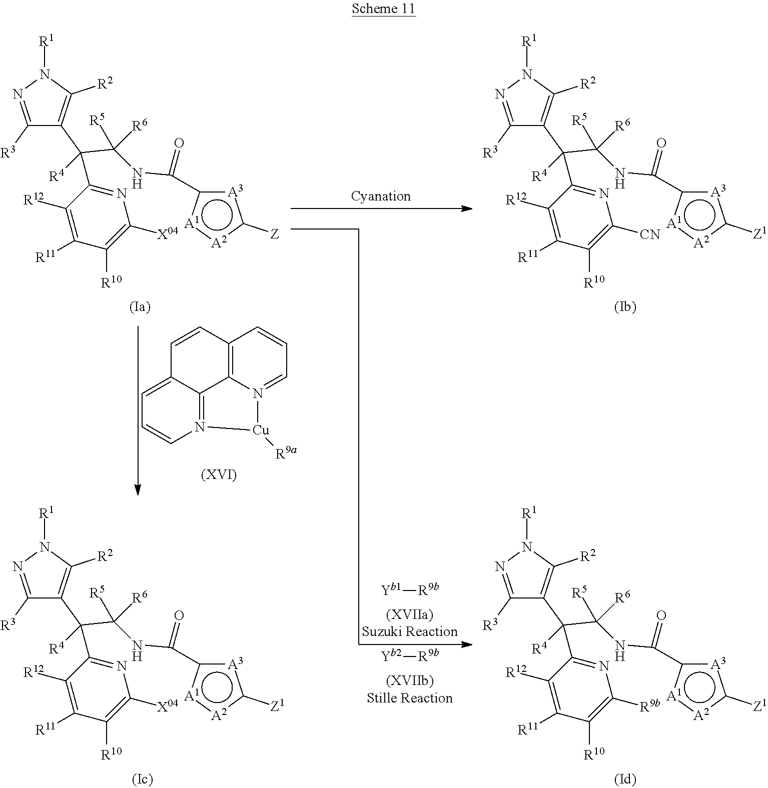

As shown in Scheme 11, compounds of formula (Ia), wherein B¹ is C—X⁰⁴, B² is CR¹⁰, B³ is CR¹¹, B⁴ is CR¹², and R¹, R², R³, R⁴, R⁵, R⁶, R¹⁰, R¹¹, R¹², A¹, A², A³ and Z¹ are as defined above for the compound of formula (I), and R⁹ is cyano, namely compounds of formula (Ib), can be obtained from compound of formula (Ia) by treatment with an inorganic cyanide source such as CuCN an inert solvent such as dimethylformamide (DMF) or N-methyl-2-pyrrolidone at temperatures between 0° C. and 150° C. Such reactions are well known in the literature, for example, in J. Het. Chem. 1987, 24(2), 373-6, Liebigs Ann. Chem. 1994, (10), 1049-53, and Org. Prep. Proc. Int. 1985, 17(6), 391-9. Other methods for introduction of the cyano group by substitution of a halogen atom are known in the art. See, for example, Science of Synthesis 2004, 19, 173-195.

Compounds of formula (I), wherein B¹ is CR^{9a}, B² is CR¹⁰, B³ is CR¹¹, B⁴ is CR¹², and R¹, R², R³, R⁴, R⁵, R⁶, R¹⁰, R¹¹, R¹², A¹, A², A³ and Z¹ are as defined above for the compound of formula (I), and R^{9a} is C₁-C₄ haloalkyl, namely compounds of formula (Ic), can be prepared by treating compounds of formula (Ia) wherein R¹, R², R³, R⁴, R⁵, R⁶, R¹⁰, R¹¹, R¹², A¹, A², A³ and Z¹ are as defined above for the compound of formula (I), and X⁰⁴ is halogen, preferably bromine, with compounds of formula (XVI), wherein R^{9a} is C₁-C₄ haloalkyl, in an inert solvent such as DMF or N-methyl-2-pyrrolidone at temperatures between room temperature and 150° C. Such reactions are known in the literature (Org. Lett. 2014, 16(6). 1744-1747). Compounds of formula (I), wherein B¹ is CR^{9b}, B² is CR¹⁰, B³ is CR¹¹, B⁴ is CR¹², and R¹, R², R³, R⁴, R⁵, R⁶, R¹⁰, R¹¹, R¹², A¹, A², A³ and Z¹ are as defined above for the compound of formula (I), and R^{9b} is phenyl, 5- or 6-membered heteroaryl or C₃-C₆ cycloalkyl, wherein the 5- or 6-membered heteroaryl comprises 1, 2, 3 or 4 heteroatoms individually selected from N, O and S, and wherein any of said phenyl, 5- or 6-membered heteroaryl and C₃-C₆ cycloalkyl are unsubstituted or substituted by 1, 2 or 3 substituents independently selected from halogen, cyano, C₁-C₄ alkyl, C₁-C₄ haloalkyl and C₁-C₄ alkoxy, namely compounds of formula (Id), can prepared (as shown in Scheme 11) by a Suzuki reaction, which involves, for example, reacting compounds of formula (Ia), wherein X⁰⁴ is a leaving group like, for example, chlorine, bromine or iodine, with compounds of formula (XVIIa), wherein Y^{b1} can be a boron-derived functional group, as for example B(OH)₂ or B(OR_{b1})₂, wherein R_{b1} can be a C₁-C₄ alkyl group or the two groups OR^{b1} can form, together with the boron atom, a five membered ring, as for example a pinacol boronic ester. The reaction is catalyzed by a palladium-based catalyst, for example tetrakis(triphenylphosphine)-palladium or (1,1′-bis(diphenylphosphino)-ferrocene)dichloropalladium-dichloromethane (1:1 complex), in presence of a base, like sodium carbonate or cesium fluoride, in a solvent or a solvent mixture, like, for example, a mixture of 1,2-dimethoxyethane and water, or dioxane and water, or methyl tetrahydrofuran and water, preferably under inert atmosphere. The reaction temperature can preferentially range from room temperature to the boiling point of the reaction mixture. Such Suzuki reactions are well known to those skilled in the art and have been reviewed, for example, in J. Organomet. Chem. 1999, 576, 147-168.

Alternatively, compounds of formula (Ic) can be prepared by a Stille reaction of compounds of formula (XVIIb), wherein Y_{b2} is a trialkyl tin derivative, preferably tri-n-butyl tin, with compounds of formula (Ia). Such Stille reactions are carried out in the presence of a palladium catalyst, for example tetrakis(triphenylphosphine)palladium(0) or (1,1′-bis(diphenylphosphino)-ferrocene)dichloropalladium-dichloromethane (1:1 complex), in an inert solvent such as DMF, acetonitrile, or dioxane, optionally in the presence of an additive, such as cesium fluoride, or lithium chloride, and optionally in the presence of a further catalyst, for example copper(I) iodide. Such Stille couplings are also well known to those skilled in the art, and have been described, for example, in J. Org. Chem. 2005, 70, 8601-8604, J. Org. Chem. 2009, 74, 5599-5602, and Angew. Chem. Int. Ed. 2004, 43, 1132-1136. A large number of compounds of formula (XVIIa) and (XVIIb) are commercially available or can be prepared by those skilled in the art. Further compounds available from compounds of formula (Ia) are shown in Scheme 12.

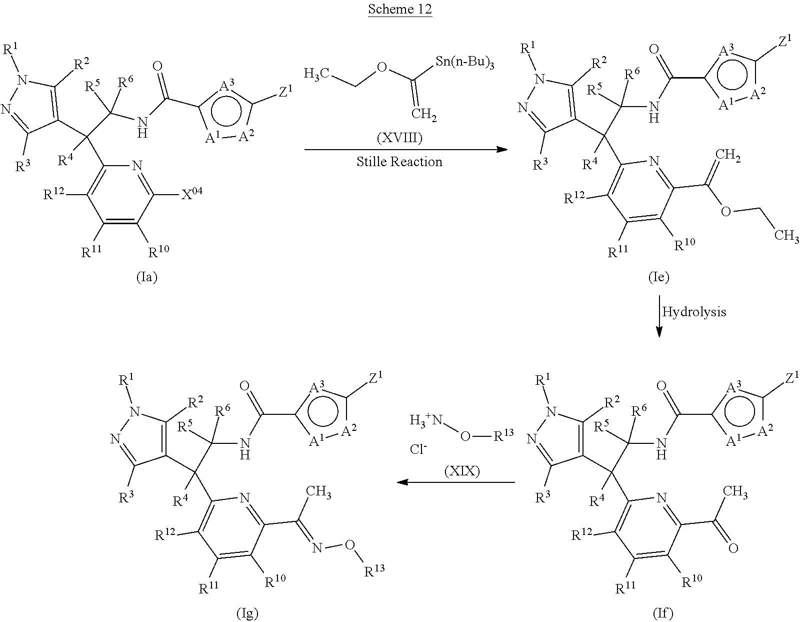

As shown in Scheme 12, compounds of formula (I), wherein B¹ is CX⁰⁴, B² is CR¹⁰, B³ is CR¹¹, B⁴ is CR¹², and R¹, R², R³, R⁴, R⁵, R⁶, R¹⁰, R¹¹, R¹², A¹, A², A³ and Z¹ are as defined above for the compound of formula (I), and X⁰⁴ is a leaving group like, for example, chlorine, bromine or iodine, namely compounds of formula (Ia), can be treated with compounds of formula (XVIII) under Stille reactions conditions to give compounds of formula (Ie). Compounds of formula (Ie) can be isolated, or directly hydrolysed under aqueous acidic conditions to give compounds of formula (If. Such reactions are known in the literature and have been described, for example, in Synthesis 2001, (10), 1551-1555, and Tetrahedron 2001, 57(13), 2507-2514. Compounds of formula (If) can be converted to compound of formula (Ig), wherein R¹, R², R³, R⁴, R⁵, R⁶, R¹⁰, R¹¹, R¹², A¹, A², A³ and Z¹ are as defined above for the compound of formula (I), and R¹³ is hydrogen or C₁-C₄ alkyl by treatment of compounds of formula (If with compounds of formula (XIX) (or a salt thereof), wherein R¹³ is hydrogen or C₁-C₄ alkyl, in an inert solvent such as methanol, ethanol, tetrahydrofuran, methyl, optionally in the presence of an inorganic base such as sodium or potassium carbonate, or organic bases such as triethylamine and the like. Many examples for the preparation of such oximes are known in the literature (see, for example, Molecules 2019, 24, 2470 and references cited therein) and are well known to those skilled in the art.

Further compounds that can be prepared from compounds of formula (I), wherein R¹, R², R³, R⁴, R⁵, R⁶, R¹⁰, R¹¹, R¹², A¹, A², A³ and Z¹ are as defined above for the compound of formula (I), and X⁰⁴ is a leaving group like, for example, chlorine, bromine or iodine, namely compounds of formula (Ia), are shown in Scheme 13.

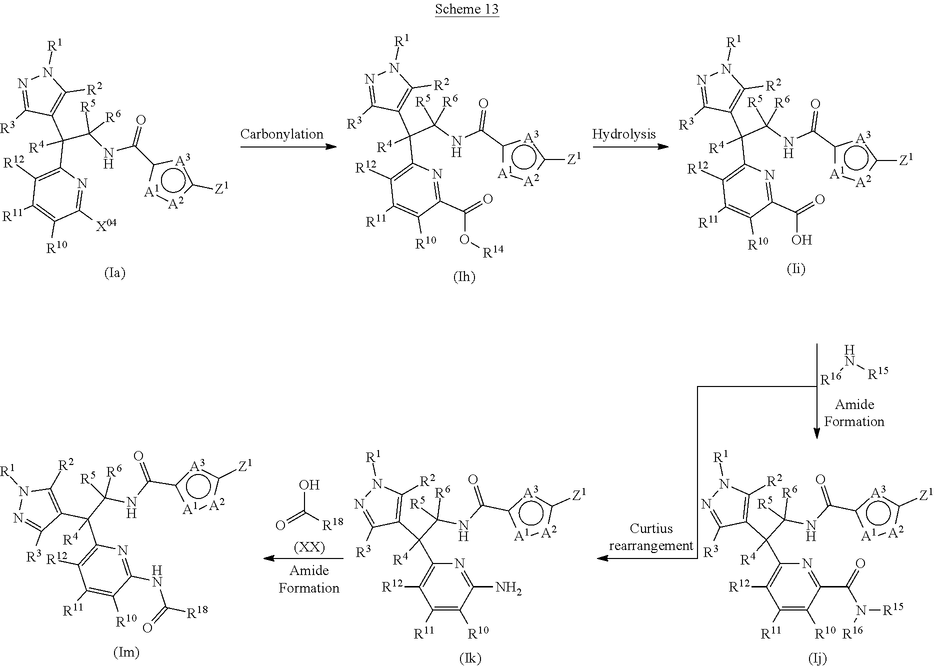

As shown in Scheme 13, compounds of formula (Ia) can be carbonylated to give compounds of formula (I), wherein R¹, R², R³, R⁴, R⁵, R⁶, R¹⁰, R¹¹, R¹², A¹, A², A³ and Z¹ are as defined above for the compound of formula (I), and R¹⁴ is C₁-C₄ alkyl, namely compounds of formula (Ih). In such alkoxycarbonylations, compounds of formula (Ia) are reacted with carbon monoxide, usually under pressure, in the presence of metal catalyst such as a palladium catalyst (for example, palladium(II) acetate, [1,1′-bis(diphenylphosphino)ferrocene]palladium(II) dichloride Pd(dppf)Cl₂, bis(triphenylphosphine)palladium(II) dichloride PdCl₂(PPh₃)₂ or bis(diphenylphosphino)propane]palladium(II) PdCl₂(dippp)), optionally in the presence of a phosphine ligand such as triphenylphosphine or 1,1′-bis(diphenylphosphino)ferrocene, in an alcohol R¹⁴OH solvent (typically methanol or ethanol), wherein R¹⁴ is C₁-C₄ alkyl, optionally in the presence of a co-solvent (e.g., toluene, dioxane or N,N-dimethylformamide), and preferably in the presence of a base, such as for example trimethylamine, at temperatures between 20° C. and 200° C., preferably between 50° C. and 180° C. Such carbonylation reactions are well known to those skilled in the art and also in the literature (see J. Org. Chem. 2008, 73, 7102-7107, and references cited therein). Such compounds of formula (Ih) can be easily saponified to compounds of formula (Ii) under conditions known to those skilled in the art, for example conditions such as aqueous sodium, potassium or lithium hydroxide in methanol, ethanol, tetrahydrofuran or dioxane at room temperature, or up to refluxing conditions. Alternatively, treating ester compounds of formula (Ih) with halide anions, preferably chloride anions, originating from, for example, lithium chloride (or alternatively, sodium or potassium chloride), in solvents such as N,N-dimethylformamide, N,N-dimethylacetamide or N-methyl-2-pyrrolidone, may also generate the carboxylic acid compounds of formula (Ii). The reaction temperatures for such an O-demethylation range preferably from 20° C. to the boiling point of the reaction mixture, or the reaction may be performed under microwave irradiation. Compounds of formula (Ii) can be converted to amides of formula (Ij), wherein R¹, R², R³, R⁴, R⁵, R⁶, R¹⁰, R¹¹, R¹², A¹, A², A³ and Z¹ are as defined above for the compound of formula (I), and R¹⁵ and R¹⁶ are independently hydrogen or C₁-C₄ alkyl, namely compounds of formula (Ij). Such reactions usually involve activating the carboxyl group, followed by treatment with a compound R¹⁵R¹⁶NH or using coupling agents to perform the direct conversion of the acids to the amides upon treatment with compounds of formula R¹⁵R¹⁶NH. These methods have been discussed vide supra in Schemes 1 and 2.

Compounds of formula (Ii) can be converted to compounds of formula (I), wherein R¹, R², R³, R⁴, R⁵, R⁶, R¹⁰, R¹¹, R¹², A¹, A², A³ and Z¹ are as defined above for the compound of formula (I), and R⁹ is amino, namely compounds of formula (Ik), by a so called Curtius rearrangement. In the Curtius rearrangement compounds of formula (Ij) are treated with an organo-azide in the presence of a suitable base and optionally in the presence or absence of Lewis acids, in an inert solvent at temperatures between 50° C. and 200° C. Examples of organo-azide include TMSN₃, sodium azide, diphenyl phosphoryl azide or tosyl azide and suitable solvent may be toluene, xylene, THE or acetonitrile. Example of suitable Lewis acids may include Zn(OTf₂ amongst others. The isocyanates formed in the rearrangement react with water to form carbamates which decarboxylate under the reaction conditions to the corresponding amines of formula (Ik). Alternatively, the reactions can be carried out in alcohols, e.g., t-butyl alcohol, allowing the t-butyl carbamates to be isolated. These in turn can be cleaved in a separate step by methods known to those skilled in the art with acids (such as trifluoroacetic acid) to yield compounds of formula (Ik). Examples of such Curtius reactions have been reported, for example, in Org. Lett. 2005, 7, 4107-4110, J. Med. Chem. 2006, 49(12), 3614-3627, and Tetrahedron 1974, 30, 2151-2157. Compounds of formula (Ik) so obtained can be amidated to compounds of formula (Im), wherein R¹, R², R³, R⁴, R⁵, R⁶, R¹⁰, R¹¹, R¹², A¹, A², A³ and Z¹ are as defined above for the compound of formula (I), and R¹⁸ is C₁-C₄ alkyl by treatment with compounds of formula (XX) according to the amidation methods described vide supra. Those skilled in the art will recognize that such chemistry can be applied to any of the compounds of formula (I), at the positions R⁹, R¹⁰, R¹¹ or R¹² when the later groups are a leaving group such as a halogen atom.

Compounds of formula (VI), (VII), (VIII), (IX), (XI), (XII), (XVI), (XVIIa), (XVIIb), (XIX), and (XX) are easily prepared by those skilled in the art or can be purchased.

Compounds of formula (II), wherein A¹ is N, A² is O, A³ is CH, and Z¹ are as described under formula (I) namely compounds of formula (IIc) can be prepared as shown in scheme 14.

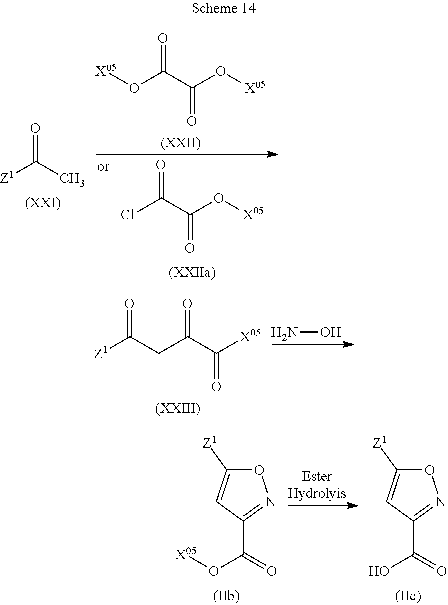

As shown in scheme 14, compounds of formula (IIc) wherein Z¹ is as described above and X⁰⁵ is C₁-C₄ alkyl may be prepared by hydrolysis of compounds of formula (IIb) by treatment with, for example, an alkaline earth metal hydroxide in water, or with a water miscible organic solvent, such as THF, methanol, ethanol and the like. Such ester hydrolyses are well known to those skilled in the art. Compounds of formula (IIb) can be obtained by the treatment of compounds of formula (XXIII) wherein Z¹ is as defined above for the compound of formula (I) and X⁰⁵ is C₁-C₄ alkyl with hydroxylamine hydrochloride in a polar solvent, for example ethanol and optionally in the presence of a base, e.g., triethyl amine, K₂CO₃ and the like.

Compounds of formula (XXIII) are prepared by reaction of compounds of formula (XXI), wherein Z¹ is defined above for the compound of formula (I), with compounds of formula (XXII) or compounds of formula (XXIIa), wherein X⁰⁵ is C₁-C₄ alkyl in presence of base such potassium tert-butoxide, sodium hydride or Lithium bis(trimethylsilyl)amide in a solvent such tetrahydrofuran or toluene. Similar reaction sequences to those described in scheme 14 to prepare compounds of formula (IIb) and (IIc) have been described in for example CN111072582 and WO2019/195810, and WO2018/019929. Further reaction conditions for preparation of compounds of formula (XXIII) are described in for example Bioorg. Med. Chem. 2016, 109, 350-359.

Compounds of formula (II) wherein A¹ is O, A² and A³ are N, and Z¹ is as previously described, namely compounds of formula (IIe) can be prepared as shown in scheme 15.

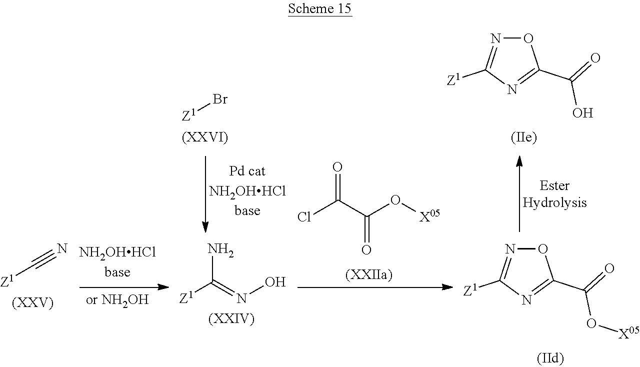

As shown in scheme 15, compounds of formula (IIe) are obtained by ester hydrolysis of compounds of formula (IId) as described previously in scheme 14. Compounds of formula (IId), wherein Z¹ is as defined under formula (I) and X⁰⁵ is C₁-C₄ alkyl are prepared by reaction of compounds of formula (XXIIa) with compounds of formula (XXIV), optionally in the presence of base, for example pyridine or triethylamine, in a solvent such as acetonitrile, chloroform or tetrahydrofuran. Similar reactions have been reported in for example Bioorg. & Med. Chem. 2016, 24(22), 5693-5701 and CN114933573. Compounds of formula (XXIV) wherein Z¹ is as defined above for the compound of formula (I), can be obtained by treatment of compounds of formula (XXV) with hydroxylamine hydrochloride in presence of base such K₂CO₃ or Na₂CO₃ in a polar solvent for example ethanol. The reaction can also be performed without base using a solution of hydroxylamine. Such reactions have been described in, for example, Bioorg. & Med. Chem. Lett. 2020, 30(21), 127508 and Bioorg. Med. Chem. Lett. 2016, 26(23), 5679-5684.

Compounds of formula (XXIV) wherein Z¹ is as defined above for the compound of formula (I), may be also prepared by “one-pot” synthesis via Pd-catalyzed cyanation and amidoximation of compound of formula (XXVI) wherein Z¹ is a defined above for the compound of formula (I) using potassium ferrocyanide trihydrate and hydroxylamine hydrochloride as described in for example Org. & Biomol. Chem. 2015, 13(9), 2541-2545.

Compounds of formula (II) wherein A¹ and A² are N, and A³ is O, and Z¹ is as previously described, (namely compounds of formula (IIg)), and A¹ and A² are N, and A³ is S, and Z¹ is as previously described (namely compounds of formula (IIi)) can be prepared as shown in scheme 16.

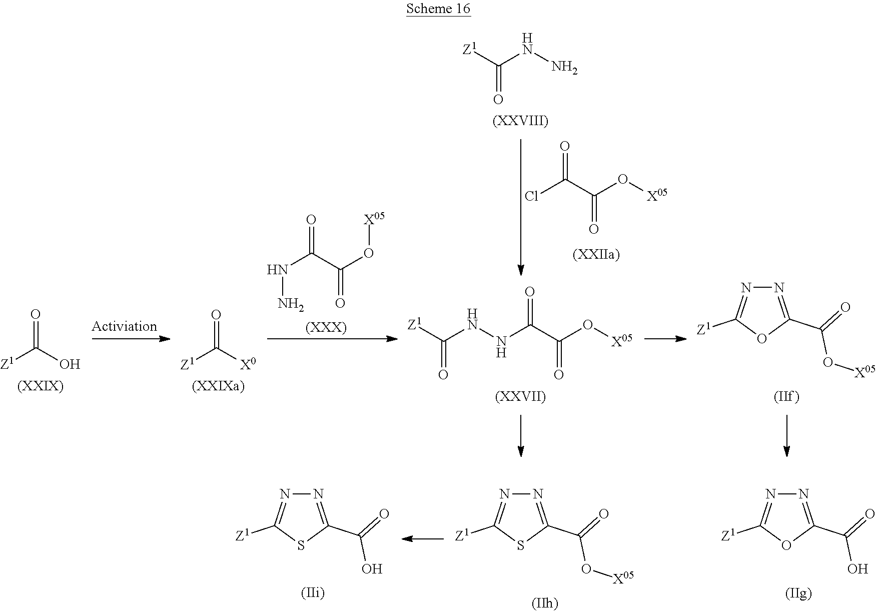

As shown in scheme 16, compounds of formula (IIg) and (Ili) are obtained by ester hydrolysis of (IIf) and (IIg), respectively. In the latter compounds, X⁰⁵ and Z¹ are as previously described. Compounds of formula (IIf) are obtained from compounds of formula (IIf) are obtained by dehydration of compounds of formula (XXVII). Compounds of formula (XXVII) are obtained by acylation of hydrazides of formula (XXVIII) with compounds of formula (XXIIa). Such sequences of reactions to produce oxadiazoles is well known to those skilled in the art. Similar reactions are described in Bioorg. Med. Chem. Lett. 2005, 15, 1423-1428 and WO2006/044617. Compounds of formula (XXVII) can also be prepared by reaction of activated carboxylic acids of formula (XXIXa), wherein Z¹ and X⁰⁵ are as described as previously and in Scheme 1 respectively with compounds of formula (XXX). Compounds of formula (XXIXa) can be prepared from the corresponding acids of formula (XXIX) as described in scheme 1. Such reactions are described for example in, for example, J. Prakt. Chem. 1985, 327, 109-116. Compounds of formula (IIh) can also be prepared from the common intermediate of formula (XXVII), wherein Z¹ and X⁰⁵ are as previously described, by treatment with Lawesson's reagent or phosphorous pentasulfide neat, or in inert solvents such as toluene or xylene. Similar reactions are known in the literature (see for example WO2010/006713, WO2009/149858 and J. Org Chem. 1961, 26, 4410-12).

Compounds of formula (II) wherein A¹ is O, A² is N, A³ is methine, and Z¹ is as previously described, namely compounds of formula (IIk), can be prepared, for example, as shown in scheme 17.

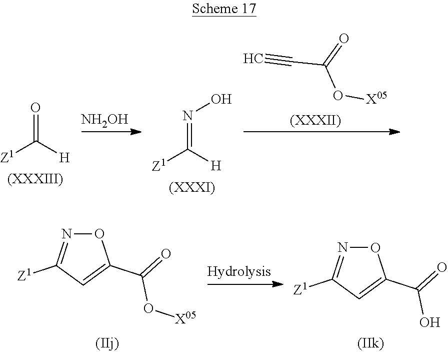

As shown in scheme 17, compounds of formula (IIk) are readily obtained by hydrolysis of esters of formula (IIj) by methods known to those skilled in the art and described vide supra. Compounds of formula (IIj) can be obtained by reaction of compounds of formula (XXXI) with compounds of formula (XXXII) in the presence of an oxidizing agent, for example (diacetoxyiodo)benzene or N-chlorosuccinimide, in an inert solvent such as methanol or DMF respectively. Such reaction sequences have been described. for example, in J. Het. Chem. 2013, 50(4), 774-780 and J. Chin. Chem. Soc. 2007, 54(3), 643-652. Compounds of formula (XXXI) are readily prepared from compounds of formula (XXXIII), wherein Z1 is as described under formula (I), by treatment with hydroxylamine under conditions well known to those skilled in the art.

A compound of formula (I) as defined in any of the embodiments of the present invention can be converted in a manner known per se into another compound as defined in any of the embodiments of the present invention by replacing one or more substituents of the starting compound in the customary manner by (an)other substituent(s) according to the invention. Those skilled in the art will also appreciate that compounds of formula (I) can be further transformed to further derivatives of formula (I) by, for example, alkylation, nucleophilic substitution, elimination, C—C-bond forming reactions in the presence of metal catalysts, heteroatom-carbon bond formation in the presence of metal catalysts, oxidation, and reduction.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula (I) may be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (I), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, or diastereomer mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and herein below, even when stereochemical details are not mentioned specifically in each case.

Diastereomeric mixtures or racemic mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is com-plexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

As an example, compounds with more than one asymmetric carbon atoms may exist in diastereomeric forms which can be optionally separated using for example supercritical fluid chromatography (SFC) chromatography with chiral columns. Such diastereomers can show a different fungicidal activity profile, but all isomers and diastereomers form part of this invention.

The compounds of formula (I) have at least two chiral carbon atoms, (two stereocenters, wherein the star (*) indicates the chiral carbon atom), such there are at least four stereoisomers available. These at least four stereoisomers consist of two sets of enantiomers.

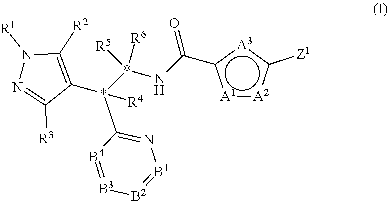

For compounds of formula (I), wherein R¹, R², R³, R⁴, R⁵, R⁶, B¹, B², B³, B⁴, A¹, A², A³, A⁴ and Z¹ are as defined for the compounds of formula (I) and wherein R⁴, and at least one of R⁵ and R⁶ is not hydrogen, the relationship between enantiomers and diastereomers is illustrated in Scheme 18.

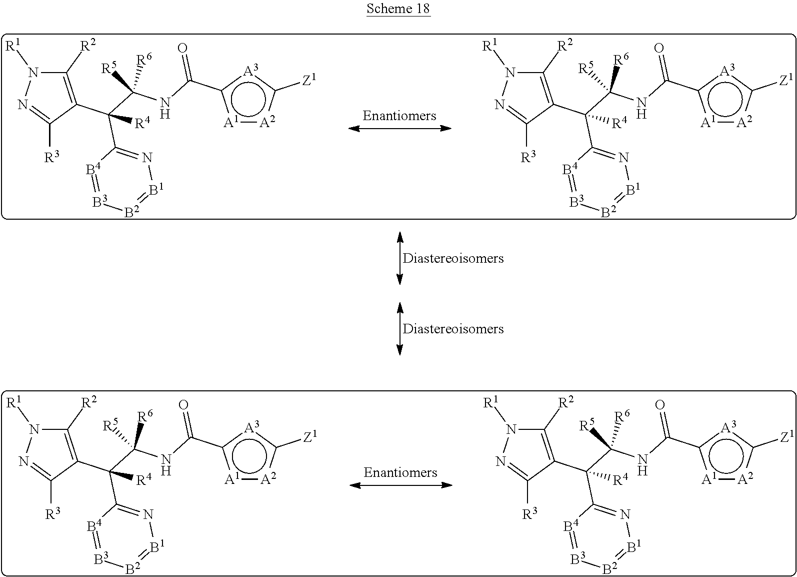

A person skilled in the art is well aware that these diastereomers and enantiomers of formula (I) (as shown in Scheme 18) wherein R¹, R², R³, R⁴, R⁵, R⁶, B¹, B², B³, B⁴, A¹, A², A³, A⁴ and Z¹ are as defined for formula (I) and wherein R⁴, and at least one of R⁵ and R⁶ is not hydrogen, are within the scope of the invention.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

The term “compounds of formula (I)” refers to component A.

The term “fungicide” as used herein means a compound that controls, modifies, or prevents the growth of fungi.

The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

The term “plants” refers to all physical parts of a plant, including seeds, seedlings, saplings, roots, tubers, stems, stalks, foliage, and fruits.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The term “locus” as used herein means fields in or on which plants are growing, or where seeds of cultivated plants are sown, or where seed will be placed into the soil. It includes soil, seeds, and seedlings, as well as established vegetation.

The term “g a.i./ha” as used herein refer to the application rate given in gramm [g] of active ingredient [a.i.] per unit of surface [ha]. The unit hectare (symbol ha) is the metric unit of area that equals a square with 100 m side (1 hm²) or 10,000 square meters. Hectare is a commonly used unit of area in the metric system.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, by the use of known techniques and by observing results obtained under analogous circumstances. In determining the effective amount a number of factors are considered including, but not limited to: the type of plant or derived product to be applied; the pest to be controlled and its lifecycle; the particular compound applied; the type of application; and other relevant circumstances.

As used herein, the term “room temperature” or “RT” or “rt” refer to a temperature of about 15° C. to about 35° C. For example, rt can refer to a temperature of about 20° C. to about 30° C.

Throughout this document the expression “composition” stands for the various mixtures or combinations of components (A) and (B) (including the above-defined embodiments), for example in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the components (A) and (B) is not essential for working the present invention.

The composition according to the invention is effective against harmful microorganisms, such as microorganisms, that cause phytopathogenic diseases, in particular against phytopathogenic fungi and bacteria.

The composition of the invention may be used to control plant diseases caused by a broad spectrum of fungal plant pathogens in the Basidiomycete, Ascomycete, Oomycete and/or Deuteromycete, Blastocladiomycete, Chrytidiomycete, Glomeromycete and/or Mucoromycete classes.

The composition is effective in controlling a broad spectrum of plant diseases, such as foliar pathogens of ornamental, turf, vegetable, field, cereal, and fruit crops.

These pathogens may include:

Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola;

Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f.sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae;

Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-virginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries;

Blastocladiomycetes, such as Physoderma maydis;

Mucoromycetes, such as Choanephora cucurbitarum; Mucor spp.; Rhizopus arrhizus;
as well as diseases caused by other species and genera closely related to those listed above.

In addition to their fungicidal activity, the compositions may also have activity against bacteria such as Erwinia amylovora, Erwinia caratovora, Xanthomonas campestris, Pseudomonas syringae, Strptomyces scabies and other related species as well as certain protozoa.

The composition according to the invention is particularly effective against phytopathogenic fungi belonging to the following classes: Ascomycetes (e.g., Venturia, Podosphaera, Erysiphe, Monilinia, Mycosphaerella, Uncinula); Basidiomycetes (e.g., the genus Hemileia, Rhizoctonia, Phakopsora, Puccinia, Ustilago, Tilletia); Fungi imperfecti (also known as Deuteromycetes; e.g., Botrytis, Helminthosporium, Rhynchosporium, Fusarium, Septoria, Cercospora, Alternaria, Pyricularia and Pseudocercosporella); Oomycetes (e.g., Phytophthora, Peronospora, Pseudoperonospora, Albugo, Bremia, Pythium, Pseudosclerospora, Plasmopara).

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g., improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g., imazamox, by conventional methods of breeding is Clearfield® summer canola. Examples of crops that have been rendered tolerant to herbicides by genetic engineering methods include e.g., glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 6-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus. Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CrylF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIAtoxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as 6-endotoxins, e.g., Cry1Ab, Cry1Ac, Cry1F, Cry1Fa2, Cry2Ab, Cry3A, Cry3Bb1 or Cry9C, or vegetative insecticidal proteins (Vip), e.g., Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plant tolerance to harmful insects. Such insects can occur in any taxonomic group of insects but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a CryIAb toxin); YieldGard Rootworm® (maize variety that expresses a Cry3Bb1 toxin); YieldGard Plus® (maize variety that expresses a CryIAb and a Cry3Bb1 toxin); Starlink® (maize variety that expresses a Cry9C toxin); Herculex I® (maize variety that expresses a Cryl Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a CryIAb toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstakiwhich brings about tolerance to certain Lepidoptera, include the European corn borer.

Additionally, to date, no cross-resistance has been observed between the composition comprising a mixture of components (A) and (B) and any fungicidal solutions used to control phytopathogenic fungi such as Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, including Diaporthe miriciae (also known as Diaporthe ueckeri or Diaporthe ueckerae), Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp., in particular, Zymoseptoria tritici, Puccinia recondita, Puccinia striiformis, Erysiphe graminis, Uncinula necator, Sphaerotheca fuliginea, Leveillula taurica, Phakopsora pachyrhizi, Pyricularia oryzae, Alternaria solani, Alternaria alternata, Mycosphaerella fijiensis, Colletotrichum lagenarium, Didymella bryoniae, Ascochyta pisii, Verticillium dahliae, Pyrenophora teres, Cercospora beticola, Ramularia collo-cygni, Botrytis cinerea, Sclerotinia sclerotiorum, Monilinia laxa, Monographella nivalis, and Venturia inaequalis.

Indeed, fungicidal-resistant strains in any of the species as outlined above have been reported in the scientific literature, with strains resistant to one or more fungicides from at least one of the following fungicidal modes of action classes: quinone-outside-inhibitors (Qol), quinone-inside-inhibitors (Qil), succinate dehydrogenase inhibitors (SDHI) and sterol demethylation-inhibitors (DMI). Such fungicidal-resistant strains may contain:

A mutation in the mitochondrial cytochrome b gene conferring resistance to Qo inhibitors, wherein the mutation is G143A, F129L or G137R. See for example: Gisi et al., Pest Manag Sci. 2000, 56, 833-841, Lucas, Pestic Outlook 2003, 14(6), 268-70, Fraaije et al., Phytopathol 2005, 95(8), 933-41, Sierotzki et al., Pest Manag. Sci. 2007, 63(3), 225-233 (2007), Semar et al., J. Plant Dis. Prot. 2007, (3), 117-119; and Pasche et al., J. Crop Prot. 2008, 27(3-5), 427-435 (2008).

A mutation in the mitochondrial cytochrome b gene conferring resistance to Qi inhibitors, wherein the mutation is G37A/C/D/SN or N31 K. See for example: Meunier et al., Pest Manag Sci. 2019; 75: 2107-2114, Young et al., Pest Manag Sci. 2018; 74(2): 489-498, Walker et al., Environ. Microbiol. 2021 (https://doi.org/10.1111/1462-2920.15760.)

A mutation in the genes encoding the SdhB,C,D subunits conferring resistance to SDHI inhibitors wherein the mutation is in the following major pathogens:

Botrytis cinerea: B—P225H/L/TY/F, B—N2301, B-H272LY/R, C—P80H/L, C—N87S;

Alternaria solani: B—H278R/Y, C—H134R/Q, D-D123E, D-H133R and C—H134R;

Zymoseptoria tritici: sdhB: N225T, N225I, R265P, T268I, T268A. In sdhC: T79N, T791, W80S, W80A, A84F, N86S, N86A, P127A, R151M/S/T/G, R151S, R151T, H152RY, V166M, T168R. In sdhD: 150F, M114V, D129G, T20P+K186R;

Pyrenophora teres: In sdhB: S66P, N235I, H277Y. In sdhC: K49E, R64K, N75S, G79R, H134R, S135R. In sdhD: D124E, H134R, G138V, D145G;

Ramularia collo-cygni: In sdhB: N224T, T267I. In sdhC: N87S, G91R, H146R/L, G171D, H153R;

Phakopsora pachyrhizi: C-186F;

Sclerotinia sclerotiorum: In sdhB: H273Y. In sdhC: G91R, H146R. In sdhD: T108K, H132R, G150R.

Major source of information is www.frac.info, Sierotzki and Scalliet, Phytopathology (2013) 103(9): 880-887 and Simões et al., J. Plant Dis. Prot. 2018, 125: 21-2.

A mutation or combination of mutations in the CYP51 gene conferring resistance to DMI inhibitors wherein the mutations are: L50S, D134G, V136A/C, Y137F, S188N, A379G, I381V, deletion 459-460, Y461H/S, N513K, S524T. Major source of information is www.frac.info, Cools et al., Plant Pathol. 2013, 62: 36-42 and Schmitz H K et al., Pest Manag. Sci. 2014, 70: 378-388.

Thus, in a preferred embodiment, the compositions according to the present invention comprising a mixture of components (A) and (B), are used to control fungal strains which are resistant to one or more fungicides from any of the following fungicidal MoA classes: quinone-outside-inhibitors (Qol), quinone-inside-inhibitors (Qil), succinate dehydrogenase inhibitors (SDHI) and sterol demethylation-inhibitors (DMI).

Compositions of this invention, including all of the above disclosed embodiments and preferred examples thereof, can be mixed with one or more further pesticides including further fungicides, insecticides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection.

Examples of such agricultural protectants with which the composition of this invention can be formulated are: Fungicides such as etridiazole, fluazinam, benalaxyl, benalaxyl-M (kiralaxyl), furalaxyl, metalaxyl, metalaxyl-M (mefenoxam), dodicin, N′-(2,5-dimethyl-4-phenoxy-phenyl)-N-ethyl-N-methyl-formamidine, N′-[4-(4,5-dichloro-thiazol-2-yloxy)-2,5-dimethyl-phenyl]-N-ethyl-N-methyl-formamidine, N′-[4-[[3-[(4-chlorophenyl)methyl]-1,2,4-thiadiazol-5-yl]oxy]-2,5-dimethyl-phenyl]-N-ethyl-N-methyl-formamidine, ethirimol, 3′-chloro-2-methoxy-N-[(3RS)-tetrahydro-2-oxofuran-3-yl]acet-2′,6′-xylidide (clozylacon), cyprodinil, mepanipyrim, pyrimethanil, dithianon, aureofungin, blasticidin-S, biphenyl, chloroneb, dicloran, benzovindiflupyr, pydiflumetofen, hexachlorobenzene, quintozene, tecnazene (TCNB), tolclofos-methyl, metrafenone, 2,6-dichloro-N-(4-trifluoromethylbenzyl)-benzamide, fluopicolide (flupicolide), tioxymid, flusulfamide, benomyl, carbendazim, carbendazim chlorhydrate, chlorfenazole, fuberidazole, thiabendazole, thiophanate-methyl, benthiavalicarb, chlobenthiazone, probenazole, acibenzolar, bethoxazin, pyriofenone (IKF-309), acibenzolar-S-methyl, pyribencarb (KIF-7767), butylamine, 3-iodo-2-propinyl n-butylcarbamate (IPBC), picarbutrazox, polycarbamate, propamocarb, tolprocarb, 3-(difluoromethyl)-N-(7-fluoro-1,1,3,3-tetramethyl-indan-4-yl)-1-methyl-pyrazole-4-carboxamide, diclocymet, N-[(5-chloro-2-isopropyl-phenyl)methyl]-N-cyclopropyl-3-(difluoromethyl)-5-fluoro-1-methyl-pyrazole-4-carboxamide, N-cyclopropyl-3-(difluoromethyl)-5-fluoro-N-[(2-isopropylphenyl)methyl]-1-methyl-pyrazole-4-carboxamide, carpropamid, chlorothalonil, flumorph, oxine-copper, cymoxanil, phenamacril, cyazofamid, flutianil, thicyofen, chlozolinate, iprodione, procymidone, vinclozolin, bupirimate, dinocton, dinopenton, dinobuton, dinocap, meptyldinocap, diphenylamine, phosdiphen, 2,6-dimethyl-[1,4]dithiino[2,3-c:5,6-c′]dipyrrole-1,3,5,7(2H,6H)-tetraone, azithiram, etem, ferbam, mancozeb, maneb, metam, metiram (polyram), metiram-zinc, nabam, propineb, thiram, vapam (metam sodium), zineb, ziram, dithioether, isoprothiolane, ethaboxam, fosetyl, phosetyl-AI (fosetyl-al), methyl bromide, methyl iodide, methyl isothiocyanate, cyclafuramid, fenfuram, validamycin, streptomycin, (2RS)-2-bromo-2-(bromomethyl)glutaronitrile (bromothalonil), dodine, doguadine, guazatine, iminoctadine, iminoctadine triacetate, 2,4-D, 2,4-DB, kasugamycin, dimethirimol, fenhexamid, hymexazole, hydroxyisoxazole imazalil, imazalil sulphate, oxpoconazole, pefurazoate, prochloraz, triflumizole, fenamidone, Bordeaux mixture, calcium polysulfide, copper acetate, copper carbonate, copper hydroxide, copper naphthenate, copper oleate, copper oxychloride, copper oxyquinolate, copper silicate, copper sulphate, copper tallate, cuprous oxide, sulphur, carbaryl, phthalide (fthalide), dingjunezuo (Jun Si Qi), oxathiapiprolin, fluoroimide, mandipropamid, KSF-1002, benzamorf, dimethomorph, fenpropimorph, tridemorph, dodemorph, diethofencarb, fentin acetate, fentin hydroxide, carboxin, oxycarboxin, drazoxolon, famoxadone, m-phenylphenol, p-phenylphenol, tribromophenol (TBP), 2-[2-[(7,8-difluoro-2-methyl-3-quinolyl)oxy]-6-fluoro-phenyl]propan-2-ol 2-[2-fluoro-6-[(8-fluoro-2-methyl-3-quinolyl)oxy]phenyl]propan-2-ol, cyflufenamid, ofurace, oxadixyl, flutolanil, mepronil, isofetamid, fenpiclonil, fludioxonil, pencycuron, edifenphos, iprobenfos, pyrazophos, phosphorus acids, tecloftalam, captafol, captan, ditalimfos, triforine, fenpropidin, piperalin, osthol, 1-methylcyclopropene, 4-CPA, chlormequat, clofencet, dichlorprop, dimethipin, endothal, ethephon, flumetralin, forchlorfenuron, gibberellic acid, gibberellins, hymexazol, maleic hydrazide, mepiquat, naphthalene acetamide, paclobutrazol, prohexadione, prohexadione-calcium, thidiazuron, tribufos (tributyl phosphorotrithioate), trinexapac, uniconazole, α-naphthalene acetic acid, polyoxin D (polyoxrim), BLAD, chitosan, fenoxanil, folpet, 3-(difluoromethyl)-N-methoxy-1-methyl-N-[1-methyl-2-(2,4,6-trichlorophenyl)ethyl]pyrazole-4-carboxamide, bixafen, fluxapyroxad, furametpyr, isopyrazam, penflufen, penthiopyrad, sedaxane, fenpyrazamine, diclomezine, pyrifenox, boscalid, fluopyram, diflumetorim, fenarimol, 5-fluoro-2-(p-tolylmethoxy)pyrimidin-4-amine, ferimzone, dimetachlone (dimethaclone), pyroquilon, proquinazid, ethoxyquin, quinoxyfen, 4,4,5-trifluoro-3,3-dimethyl-1-(3-quinolyl)isoquinoline, 4,4-difluoro-3,3-dimethyl-1-(3-quinolyl)isoquinoline, 5-fluoro-3,3,4,4-tetramethyl-1-(3-quinolyl)isoquinoline, 9-fluoro-2,2-dimethyl-5-(3-quinolyl)-3H-1,4-benzoxazepine, tebufloquin, oxolinic acid, chinomethionate (oxythioquinox, quinoxymethionate), spiroxamine, (E)-N-methyl-2-[2-(2,5-dimethylphenoxymethyl)phenyl]-2-methoxy-iminoacetamide, azoxystrobin, coumoxystrobin, dimoxystrobin, enestroburin, enoxastrobin, fenamistrobin, flufenoxystrobin, fluoxastrobin, kresoxim-methyl, mandestrobin, metaminostrobin, metominostrobin, orysastrobin, picoxystrobin, pyraclostrobin, pyrametostrobin, pyraoxystrobin, triclopyricarb, trifloxystrobin, amisulbrom, dichlofluanid, tolylfluanid, but-3-ynyl N-[6-[[(Z)-[(1-methyltetrazol-5-yl)-phenyl-methylene]amino]oxymethyl]-2-pyridyl]carbamate, dazomet, isotianil, tiadinil, thifluzamide, benthiazole (TCMTB), silthiofam, zoxamide, anilazine, tricyclazole, (rac)-cis-1-(4-chlorophenyl)-2-(1H-1,2,4-triazol-1-yl)-cycloheptanol (huanjunzuo), 1-(5-bromo-2-pyridyl)-2-(2,4-difluorophenyl)-1,1-difluoro-3-(1,2,4-triazol-1-yl)propan-2-ol 2-(1-tert-butyl)-1-(2-chlorophenyl)-3-(1,2,4-triazol-1-yl)-propan-2-ol (TCDP), azaconazole, bitertanol (biloxazol), bromuconazole, climbazole, cyproconazole, difenoconazole, dimetconazole, diniconazole, diniconazole-M, epoxiconazole, etaconazole, fenbuconazole, fluquinconazole, flusilazole, flutriafol, hexaconazole, imibenconazole, ipconazole, ipfentrifluconazole, metconazole, myclobutanil, penconazole, propiconazole, prothioconazole, simeconazole, tebuconazole, tetraconazole, triadimefon, triadimenol, triazoxide, triticonazole, mefentrifluconazole, 2-[[(1R,5S)-5-[(4-fluorophenyl)methyl]-1-hydroxy-2,2-dimethyl-cyclopentyl]methyl]-4H-1,2,4-triazole-3-thione, 2-[[3-(2-chlorophenyl)-2-(2,4-difluorophenyl)oxiran-2-yl]methyl]-4H-1,2,4-triazole-3-thione, ametoctradin (imidium), iprovalicarb, valifenalate, 2-benzyl-4-chlorophenol (Chlorophene), allyl alcohol, azafenidin, benzalkonium chloride, chloropicrin, cresol, daracide, dichlorophen (dichlorophene), difenzoquat, dipyrithione, N-(2-p-chlorobenzoylethyl)-hexaminium chloride, NNF-0721, octhilinone, oxasulfuron, propamidine and propionic acid.

Insecticides such as abamectin, acephate, acetamiprid, amidoflumet (S-1955), avermectin, azadirachtin, azinphos-methyl, bifenthrin, bifenazate, buprofezin, carbofuran, cartap, chlorantraniliprole (DPX-E2Y45), chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; Bactericides such as streptomycin;

Acaricides such as amitraz, chinomethionat, chlorobenzilate, cyenopyrafen, cyhexatin, dicofol, dienochlor, etoxazole, fenazaquin, fenbutatin oxide, fenpropathrin, fenpyroximate, hexythiazox, propargite, pyridaben and tebufenpyrad; and

Biological agents such as Bacillus thuringiensis, Bacillus thuringiensis delta endotoxin, baculovirus, and entomopathogenic bacteria, virus and fungi.

Other examples of “reference” mixture compositions are as follows (wherein the term “TX” represents a compound (according to the definition of component (A) of the compositions of the present invention) selected from compounds of formula (I), or compounds selected from (X.01), (X.02), (X.03), (X.04), (X.05), (X.06), (X.07), (X.08), (X.9), (X.10), (X.11), (X.12), (X.13), (X.14), (X.15), or (X.16), as defined in the Table X above): a compound selected from the group of substances consisting of petroleum oils+TX, 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-i 1-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8,10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B2+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocycloheX.1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl+TX, metalaxyl-M+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim--methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphecy+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N—((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1, 1-dimethylethyl)-α-[4′-(trifluoromethoxy) [1,1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO2018/202428).

The references in brackets behind the active ingredients, e.g., [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The term “compounds of formula (I)” refers to component A.

In the “reference” mixture compositions the mixtures of compounds of formula (I) (selected from (X.01), (X.02), (X.03), (X.04), (X.05), (X.06), (X.07), (X.08), (X.9), (X.10), (X.11), (X.12), (X.13), (X.14), (X.15), or (X.16), as listed in Table X (above) with active ingredients described above comprise a compound selected from Table X (above) and an active ingredient as described above preferably in a mixing ratio of from 100:1 to 1:100, especially from 50:1 to 1:50, more especially in a ratio of from 20:1 to 1:20, even more especially from 10:1 to 1:10, very especially from 5:1 to 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixture compositions as described above (both according to the invention and the “reference” mixture compositions) can be used in a method for controlling pests, which comprises applying a composition comprising a mixture as described above to the pests or their environment.

The mixtures comprising component (A), wherein said component (A) is a compound of formula (I) selected from selected from compounds of formula (I), or compounds selected from (X.01), (X.02), (X.03), (X.04), (X.05), (X.06), (X.07), (X.08), (X.9), (X.10), (X.11), (X.12), (X.13), (X.14), (X.15), or (X.16), as listed in Table X (above), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Table X (above) and the active ingredients as described above is not essential for working the present invention.

The compositions of the present invention may also be used in crop enhancement. According to the present invention, ‘crop enhancement’ means an improvement in plant vigour, an improvement in plant quality, improved tolerance to stress factors, and/or improved input use efficiency.

According to the present invention, an ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use less seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, less dead basal leaves, delay of senescence, improved vitality of the plant, increased levels of amino acids in storage tissues and/or less inputs needed (e.g., less fertiliser, water and/or labour needed). A plant with improved vigour may have an increase in any of the aforementioned traits or any combination or two or more of the aforementioned traits.

According to the present invention, an ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g., seeds, fruits, leaves, vegetables (such improved quality may manifest as improved visual appearance of the harvested material), improved carbohydrate content (e.g., increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g., improved taste) and/or improved consumer health benefits (e.g., increased levels of vitamins and anti-oxidants), improved post-harvest characteristics (e.g., enhanced shelf-life and/or storage stability, easier processability, easier extraction of compounds), more homogenous crop development (e.g., synchronised germination, flowering and/or fruiting of plants), and/or improved seed quality (e.g., for use in following seasons). A plant with improved quality may have an increase in any of the aforementioned traits or any combination or two or more of the aforementioned traits.

According to the present invention, an ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to abiotic stress factors which cause sub-optimal growing conditions such as drought (e.g., any stress which leads to a lack of water content in plants, a lack of water uptake potential or a reduction in the water supply to plants), cold exposure, heat exposure, osmotic stress, UV stress, flooding, increased salinity (e.g., in the soil), increased mineral exposure, ozone exposure, high light exposure and/or limited availability of nutrients (e.g., nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients.

According to the present invention, an ‘improved input use efficiency’ means that the plants are able to grow more effectively using given levels of inputs compared to the grown of control plants which are grown under the same conditions in the absence of the method of the invention. In particular, the inputs include, but are not limited to fertiliser (such as nitrogen, phosphorous, potassium, micronutrients), light and water. A plant with improved input use efficiency may have an improved use of any of the aforementioned inputs or any combination of two or more of the aforementioned inputs.

Other crop enhancements of the present invention include a decrease in plant height, or reduction in tillering, which are beneficial features in crops or conditions where it is desirable to have less biomass and fewer tillers. Any or all of the above crop enhancements may lead to an improved yield by improving e.g., plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g., improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g., perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example, pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The compositions of the present invention may also be used in the field of protecting storage goods against attack of fungi. According to the present invention, the term “storage goods” is understood to denote natural substances of vegetable and/or animal origin and their processed forms, which have been taken from the natural life cycle and for which long-term protection is desired. Storage goods of vegetable origin, such as plants or parts thereof, for example stalks, leaves, tubers, seeds, fruits, or grains, can be protected in the freshly harvested state or in processed form, such as pre-dried, moistened, comminuted, ground, pressed or roasted. Also falling under the definition of storage goods is timber, whether in the form of crude timber, such as construction timber, electricity pylons and barriers, or in the form of finished articles, such as furniture or objects made from wood. Storage goods of animal origin are hides, leather, furs, hairs, and the like. The composition according to the present invention can prevent disadvantageous effects such as decay, discoloration, or mold. Preferably “storage goods” is understood to denote natural substances of vegetable origin and/or their processed forms, more preferably fruits and their processed forms, such as pomes, stone fruits, soft fruits and citrus fruits and their processed forms. In another preferred embodiment of the invention “storage goods” is understood to denote wood.

Therefore, a further aspect of the present invention is a method of protecting storage goods, which comprises applying to the storage goods a composition according to the invention.

The composition of the present invention may also be used in the field of protecting technical material against attack of fungi. According to the present invention, the term “technical material” includes paper; carpets; constructions; cooling and heating systems; wallboards; ventilation and air conditioning systems and the like; preferably “technical material” is understood to denote wallboards. The composition according to the present invention can prevent disadvantageous effects such as decay, discoloration, or mold.

The composition according to the invention is generally formulated in various ways using formulation adjuvants, such as carriers, solvents and surface-active substances. The formulations can be in various physical forms, e.g., in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g., from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g., by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in microcapsules. Microcapsules contain the active ingredients in a porous carrier. This enables the active ingredients to be released into the environment in controlled amounts (e.g., slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane, or chemically modified polymers and starch xanthates, or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the formulations according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g., in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The formulations according to the invention can include an additive comprising an oil of vegetable or animal origin, a mineral oil, alkyl esters of such oils or mixtures of such oils and oil derivatives. The amount of oil additive in the formulation according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The formulations generally comprise from 0.1 to 99% by weight, especially from 0.1 to 95% by weight, of compounds of component (A) and component (B) and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline, the compositions or compounds may be applied at a rate of from 1 to 2000 l/ha, especially from 10 to 1000 l/ha.

Certain mixture compositions comprising a component (A), wherein said component (A) is a compound of formula (I) described above may show a synergistic effect. This occurs whenever the action of an active ingredient combination is greater than the sum of the actions of the individual components. The action to be expected E for a given active ingredient combination obeys the so-called COLBY formula and can be calculated as follows (COLBY, S.R. “Calculating synergistic and antagonistic responses of herbicide combination”. Weeds, Vol. 15, pages 20-22; 1967):

$$ppm = milligrams of active ingredient ( = a . i . ) per liter of spray mixture \\ X = \% action by active ingredient A ) using p ppm of active ingredient \\ Y = \% active by active ingredient B ) using q ppm of active ingredient .$$

According to COLBY, the expected (additive) action of active ingredients A)+B) using p+q ppm of active ingredient is:

$$E = X + Y - \frac{X \cdot Y}{100}$$

If the action actually observed (O) is greater than the expected action (E), then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

However, besides the actual synergistic action with respect to fungicidal activity, the composition according to the invention may also have further surprising advantageous properties. Examples of such advantageous properties that may be mentioned are: more advantageous degradability; improved toxicological and/or ecotoxicological behaviour; or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigor, and early germination.

The composition according to the invention can be applied to the phytopathogenic microorganisms, the useful plants, the locus thereof, the propagation material thereof, storage goods or technical materials threatened by microorganism attack.

The composition according to the invention may be applied before or after infection of the useful plants, the propagation material thereof, storage goods or technical materials by the microorganisms.

The amount of a composition according to the invention to be applied, will depend on various factors, such as the compounds employed; the subject of the treatment, such as, for example plants, soil or seeds; the type of treatment, such as, for example spraying, dusting or seed dressing; the purpose of the treatment, such as, for example prophylactic or therapeutic; the type of fungi to be controlled or the application time.

When applied to the useful plants component (A) is typically applied at a rate of 5 to 2000 g a.i./ha, particularly 10 to 1000 g a.i./ha, e.g., 50, 75, 100 or 200 g a.i./ha, typically in association with 1 to 5000 g a.i./ha, particularly 2 to 2000 g a.i./ha, e.g., 100, 250, 500, 800, 1000, 1500 g a.i./ha of component (B).

In agricultural practice the application rates of the composition according to the invention depend on the type of effect desired, and typically range from 20 to 4000 g of total composition per hectare.

When the composition according to the invention is used fortreating seed, rates of 0.001 to 50 g of a compound of component (A) per kg of seed, preferably from 0.01 to 10 g per kg of seed, and 0.001 to 50 g of a compound of component (B), per kg of seed, preferably from 0.01 to 10 g per kg of seed, are generally sufficient.

For the avoidance of doubt, where a literary reference, patent application, or patent, is cited within the text of this application, the entire text of said citation is herein incorporated by reference.

### EXAMPLES

The Examples which follow serve to illustrate the invention and are not meant in any way to limit the invention.

The compounds of the invention can be distinguished from known compounds by virtue of greater efficacy at low application rates, which can be verified by a person skilled in the art using the experimental procedures outlined in the Examples, using lower application rates, if necessary, for example 60 ppm, 20 ppm or 2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius and “m.p.” means melting point. LC/MS means Liquid Chromatography Mass Spectroscopy and the description of the apparatus, and the methods is as follows.

¹H NMR and ¹⁹F NMR measurements were recorded on a Bruker 400 MHz spectrometer, chemical shifts are given in ppm relevant to a TMS (¹H) and CFCl3 (¹⁹F) standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

LC-MS Method A: Spectra were recorded on a mass spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: binary pump, heated column compartment, diode-array detector and ELSD detector. Column: Waters UPLC HSS T3, 1.8 μm, 30×2.1 mm, Temperature: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min): 0.85.

LC-MS Method B: Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Extractor: V (No extractor voltage for QDa detector) Source Temperature: 120° C., Desolvation Temperature: 600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

LC-MS Method C: Spectra were recorded on a Mass Spectrometer from Agilent Technologies (6410 Triple Quadrupole mass spectrometer) equipped with an electrospray source (Polarity: positive or negative ions, MS2 Scan, Capillary: 4.00 kV, Fragmentor: 100 V, Desolvation Temperature: 350° C., Gas Flow: 11 L/min, Nebulizer Gas: 45 psi, Mass range: 110 to 1000 Da) and a 1200 Series HPLC from Agilent: quaternary pump, heated column compartment and VWD detector. Column: KINETEX EVO C18, 2.6 μm, 50×4.6 mm, Temp: 40° C., Detector VWD Wavelength: 254 nm, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

### FORMULATION EXAMPLES

### Wettable Powders

| | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether (7-8 mol | — | 2% | — |
| of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

### Powders for Dry Seed Treatment

| | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

### Emulsifiable Concentrate

| | |
|---|---|
| active ingredients | 10% |
| octylphenol polyethylene glycol ether (4-5 mol | 3% |
| of ethylene oxide) | |
| calcium dodecylbenzene sulfonate | 3% |
| castor oil polyglycol ether (35 mol of | 4% |
| ethylene oxide) | |
| Cyclohexanone | 30% |
| xylene mixture | 50% |

Emulsions of any required dilution, which can be used in plant protection, can be obtained from this concentrate by dilution with water.

| | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such dusts can also be used for dry dressings for seed.

### Extruder Granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

### Coated Granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol | 6% |
| of ethylene oxide) | |
| Sodium lignosulfonate | 10% |
| carboxymethylcellulose | 1% |
| silicone oil (in the form of a 75% emulsion | 1% |
| in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a | 0.5% |
| 20% solution in water) | |
| monoazo-pigment calcium salt | 5% |
| Silicone oil (in the form of a 75% emulsion in | 0.2% |
| water) | |
| Water | 45.3% |

The finely ground combination is intimately mixed with the adjuvants, giving a flowable concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### ABBREVIATIONS

CDCl₃ deuterated chloroform
DCC dicyclohexyl carbodiimide
DCM dichloromethane (or methylene chloride or methylene dichloride)
DMF dimethylformamide
DMSO dimethyl sulfoxide
DMSO-d6 deuterated Dimethyl sulfoxide
EtOAc ethylacetate
HCl hydrochloric acid
h/hrs hour/hours
LC-MS Liquid Chromatography Mass Spectrometry (LC-MS or LCMS)
rh relative humidity
rt room temperature
Rt retention time
ssp. subspecies
THE tetrahydrofuran

### PREPARATORY EXAMPLES

The compounds of formula (I) according to the invention may be prepared using the synthetic techniques described both above and below.

“Mp” means melting point in ° C. Free radicals represent methyl groups. ¹H NMR and ¹⁹F NMR measurements were recorded on a Bruker 400 MHz spectrometer (or 600 MHz as indicated), chemical shifts are given in ppm relevant to a TMS (¹H) and CFCl3 (¹⁹F) standard. Spectra measured in deuterated solvents as indicated. Either one of the LC-MS methods below was used to characterize the compounds. The characteristic LCMS values obtained for each compound were the retention time (“Rt”, recorded in minutes) and the measured molecular ion (M+H)⁺ or (M−H)⁻.

### Example P1: Preparation of N-[2-(6-chloro-2-pyridyl)-2-(1-methylpyrazol-4-yl)propyl]-5-(2,4-difluorophenyl)isoxazole-3-carboxamide (Compound X.01, Table P)

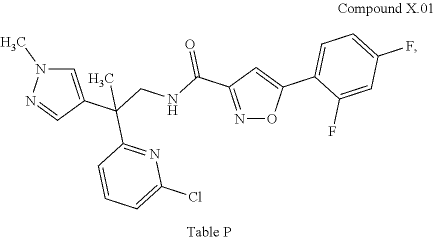 [SMILES: Cn1cc(C(C)(CNC(=O)c2cc(-c3ccc(F)cc3F)on2)c2cccc(Cl)n2)cn1]

### Step 1: Preparation of 2-(1-methylpyrazol-4-yl)propane nitrile

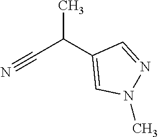

A sample of 2-(1-methyl-1 h-pyrazol-4-yl)acetonitrile (10.00 g, 78.42 mmol) was dissolved in tetrahydrofuran (314 mL) under argon and the pale-yellow solution was cooled to −78° C. To this solution was added dropwise n-butyl lithium (31 mL, 78.42 mmol) and the resulting pale brown suspension was stirred at this temperature for 25 min. After this time, iodomethane (11.24 g, 4.93 mL, 78.42 mmol) was added dropwise. The resulting brown solution was stirred at −78° C. for 5 min, allowed to warm to rt and stirred for 30 min under argon. The reaction mixture was poured into water and extracted twice with EtOAc. The combined organic layers were washed once with brine, dried over anhydrous sodium sulfate, filtered and concentrated under reduced pressure to give 2-(1-methylpyrazol-4-yl)propanenitrile as a light brown liquid. LC/MS (Method A); 136 [M+H]⁺; retention time: 0.43 min, 0.44 min, 0.55 min. ¹H NMR (400 MHz, CDCl₃) δ ppm 1.64 (d, J=6.90 Hz, 3H) 3.86-3.93 (m, 4H) 7.40 (s, 1H) 7.46 (s, 1H).

### Step 2: Preparation of 2-(6-chloro-2-pyridyl)-2-(1-methylpyrazol-4-yl)propanenitrile

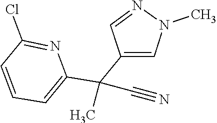 [SMILES: Cn1cc(C(C)(C#N)c2cccc(Cl)n2)cn1]

A sample of 2-(1-methylpyrazol-4-yl)propanenitrile (2.00 g, 14.80 mmol) was dissolved in tetrahydrofuran (59.18 mL) under argon to give a pale-yellow solution. The solution was cooled to −78° C. and then treated dropwise with n-butyl lithium (5.90 mL, 14.80 mmol). The resulting pale brown suspension was stirred at this temperature for 10 min before adding 2,6-dichloropyridine (2.23 g, 14.80 mmol) portion wise. The resulting brown suspension was stirred at −78° C. for 5 min, let to reach rt and stirred for 30 min under argon to give a light brown solution. The reaction mixture was poured into water and extracted twice with EtOAc. The combined organic layers were washed once with brine, dried over anhydrous sodium sulfate, filtered and concentrated under reduced pressure at 60° C. to give 3.68 g of a dark brown liquid. The crude was purified by flash chromatography with an eluent of EtOAc in cyclohexane to yield 2-(6-chloro-2-pyridyl)-2-(1-methylpyrazol-4-yl)propanenitrile (2.71 g, 74.2 yield) as a pale-yellow oil. LC/MS (method A); 247 [M+H]⁺; retention time: 0.82 min. ¹H NMR (400 MHz, CDCl₃) δ ppm 2.12 (s, 3H) 3.90 (s, 3H) 7.27-7.32 (m, 1H) 7.45 (s, 1H) 7.46-7.54 (m, 2H) 7.62-7.74 (m, 1H).

### Step 3: Preparation of 2-(6-chloro-2-pyridyl)-2-(1-methylpyrazol-4-yl)propan-1-amine

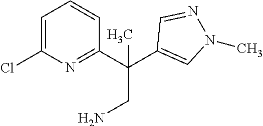 [SMILES: Cn1cc(C(C)(CN)c2cccc(Cl)n2)cn1]

A solution of 2-(6-chloro-2-pyridyl)-2-(1-methylpyrazol-4-yl)propanenitrile (2.71 g, 11.0 mmol) in tetrahydrofuran (33.0 mL) was treated dropwise with BMS (2.50 g, 3.13 mL, 33.0 mmol) at rt under argon and the resulting pale-yellow solution was stirred for 4 hr at 65° C. The reaction mixture was cooled to 0° C. before adding dropwise concentrated hydrochloric acid (7.36 mL, 44.2 mmol), and stirred for 1 hr at 50° C. The reaction mixture was cooled and treated with water. The reaction mixture was adjusted to pH 12 with NaOH 6N and extracted with EtOAc (3×20 mL). The combined organic layers were washed once with brine, dried over anhydrous sodium sulfate and concentrated under reduced pressure to give 2-(6-chloro-2-pyridyl)-2-(1-methylpyrazol-4-yl)propan-1-amine as a pale-yellow oil, that could be used in the next step without further purification. ¹H NMR (400 MHz, DMSO d6) δ ppm 1.57 (s, 3H) 2.98 (d, J=12.72 Hz, 1H) 3.16 (d, J=12.72 Hz, 1H) 3.77 (s, 3H) 7.20-7.35 (m, 3H) 7.50 (s, 1H) 7.75 (t, J=7.81 Hz, 1H).

### Step 4: Preparation of N-[2-(6-chloro-2-pyridyl)-2-(1-methylpyrazol-4-yl)propyl]-5-(2,4-difluorophenyl) isoxazole-3-carboxamide (Compound X.01. Table P)

A sample of (1-cyano-2-ethoxy-2-oxoethylidenaminooxy)dimethylamino-morpholino-carbenium-hexafluorophosphate (COMU) (0.10 g, 0.23 mmol) was added to a white suspension of 2-(6-chloro-2-pyridyl)-2-(1-methylpyrazol-4-yl)propan-1-amine (0.05 g, 0.20 mmol), 5-(2,4-difluorophenyl)isoxazole-3-carboxylic acid (0.05 g, 0.22 mmol, prepared as described in WO2018/019929) and N,N-diisopropylethylamine (0.05 g, 0.07 mL, 0.40 mmol) in 2-MeTHF (2.0 mL) under argon. After reaction completion, the reaction mixture was diluted with EtOAc, and the separated EtOAc phase washed once with 10 ml of 0.05 M aqueous HCl, once with saturated aqueous sodium bicarbonate, and then dried over anhydrous Na₂SO₄, filtered and concentrated under reduced pressure to give a pale brown oily powder. The crude mixture was first purified by flash chromatography eluting with EtOAc in cyclohexane and then by reversed phase flash chromatography eluting with acetonitrile in water to give N-[2-(6-chloro-2-pyridyl)-2-(1-methylpyrazol-4-yl)propyl]-5-(2,4-difluorophenyl)isoxazole-3-carboxamide as a white powder. LC/MS (Method A); 458 [M+H]⁺; retention time: 1.08 min. ¹H NMR (400 MHz, CDCl₃) δ ppm 1.74 (s, 3H) 3.89 (s, 3H) 4.01-4.15 (m, 2H) 6.96-7.16 (m, 4H) 7.23-7.31 (m, 3H) 7.60 (t, J=7.81 Hz, 1H) 7.94 (qd, J=8.90, 6.36 Hz, 2H).

### Example P2: Preparation of N-[2-(6-chloro-2-pyridyl)-2-(1-methylpyrazol-4-yl)propyl]-5-(2,4-difluorophenyl)-1,3,4-thiadiazole-2-carboxamide (Compound X.13, Table P)

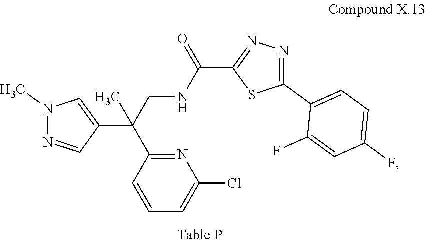 [SMILES: Cn1cc(C(C)(CNC(=O)c2nnc(-c3ccc(F)cc3F)s2)c2cccc(Cl)n2)cn1]

Step 1 to 3 are the same as for Example P1.

### Step 4: Preparation of N-[2-(6-chloro-2-pyridyl)-2-(1-methylpyrazol-4-yl)propyl]-5-(2,4-difluorophenyl)-1,3,4-thiadiazole-2-carboxamide (Compound X.13, Table P)

Trimethylaluminum (0.20 mL, 0.40 mmol) was added dropwise to a pale beige suspension of ethyl 5-(2,4-difluorophenyl)-1,3,4-thiadiazole-2-carboxylate (0.070 g, 0.26 mmol, prepared as described in WO2019/014308) in 0.4 ml of toluene under argon atmosphere (gas evolution and exothermic addition). The resulting pale brown solution was stirred at rt for 15 min. A pale brown solution of 2-(6-chloro-2-pyridyl)-2-(1-methylpyrazol-4-yl)propan-1-amine (as prepared in Example P1, step 3) (0.05 g, 0.20 mmol) in 0.4 ml of toluene was then added dropwise at rt (gas evolution). The resulting yellow solution was stirred for 2 hr at 90° C. under argon. The reaction mixture was cooled to 0° C. and quenched carefully by addition of HCl 1 M (0.25 mL). The mixture was diluted with saturated NaHCO₃ and extracted twice with EtOAc. The combined organic layers were washed once with brine, dried over anhydrous sodium sulfate, filtered and concentrated under reduced pressure to give the crude product which was purified by flash chromatography eluting with acetonitrile in water to give the title compound as a white powder. LC/MS (method A); 475 [M+H]⁺, retention time: 1.08 min. ¹H NMR (400 MHz, CDCl₃) δ ppm 1.75 (s, 3H) 3.89 (s, 3H) 4.05-4.20 (m, 2H) 7.00-7.18 (m, 3H) 7.24-7.30 (m, 3H) 7.60 (t, J=7.81 Hz, 1H) 8.31 (br t, J=6.18 Hz, 1H) 8.45 (td, J=8.54, 6.54 Hz, 1H).

### Example P3: Preparation of N-[2-(6-chloro-2-pyridyl)-2-(1-methylpyrazol-4-yl)propyl]-5-(3,5-difluoro-2-pyridyl)-1,3,4-thiadiazole-2-carboxamide (compound X.15, Table P)

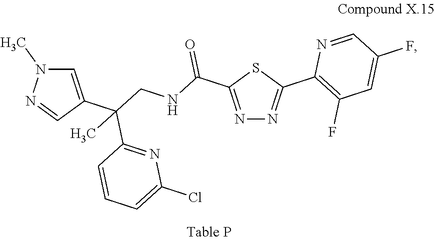 [SMILES: Cn1cc(C(C)(CNC(=O)c2nnc(-c3ncc(F)cc3F)s2)c2cccc(Cl)n2)cn1]

### Step 1 to 3: Preparation of 2-(6-chloro-2-pyridyl)-2-(1-methylpyrazol-4-yl)propan-1-amine (See Example P1, step 1 to 3)

### Step 4: Preparation of tert-butyl N-[(3,5-difluoropyridine-2-carbonyl)amino]carbamate

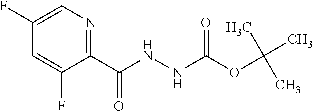 [SMILES: CC(C)(C)OC(=O)NNC(=O)c1ncc(F)cc1F]

A solution of tert-butyl N-aminocarbamate (0.82 g, 6.26 mmol) in EtOAc (12 mL) was treated dropwise with a solution of 3,5-difluoropyridine-2-carbonyl chloride (1.17 g, 6.26 mmol) in EtOAc (12 mL) at rt under argon. The reaction mixture was stirred at rt for 1 hr upon which LCMS showed reaction completion. The reaction mixture was diluted with water, extracted with EtOAc (×3), and the combined organic layers dried over sodium sulfate and concentrated under reduced pressure to yield the title compound as a pale yellow solid. LCMS (Method B): retention time 0.57 min, m/z 174 (M-99)

### Step 5: Preparation of 3,5-difluoropyridine-2-carbohydrazide

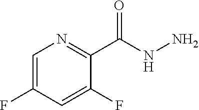 [SMILES: NNC(=O)c1ncc(F)cc1F]

A sample of tert-butyl N-[(3,5-difluoropyridine-2-carbonyl)amino]carbamate (1.7 g, 6.2 mmol) was dissolved in HCl/dioxane (16 mL, 4 mol/L) and stirred at rt for 12 hr. After completion of the reaction (monitored by LCMS and TLC), the reaction mixture was quenched with sodium bicarbonate and extracted with EtOAc (×3). The combined organic layers were dried over sodium sulfate and concentrated under reduced pressure to yield 3,5-difluoropyridine-2-carbohydrazide as a pale yellow solid. LCMS (Method B): retention time 0.16 min, m/z 174 (M+1)

### Step 6: Preparation of methyl 2-[2-(3,5-difluoropyridine-2-carbonyl)hydrazino]-2-oxo-acetate

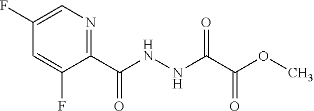 [SMILES: COC(=O)C(=O)NNC(=O)c1ncc(F)cc1F]

A solution of 3,5-difluoropyridine-2-carbohydrazide (0.7 g, 4 mmol) and triethylamine (1 mL, 10 mmol) dissolved in acetonitrile (7 mL) was cooled to 0° C. and treated dropwise with methyl oxalyl chloride (0.4 mL, 4 mmol). The resulting reaction mixture was stirred at rt for 1 hr. The reaction mixture was then diluted with water (20 mL) and extracted with EtOAc (×3). The combined organic layers were dried over sodium sulphate and concentrated under reduced pressure to obtain methyl 2-[2-(3,5-difluoropyridine-2-carbonyl)hydrazino]-2-oxo-acetate. LCMS (Method B): retention time 0.19 min, m/z 260 (M+1)

### Step 7: Preparation of methyl 5-(3,5-difluoro-2-pyridyl)-1,3,4-thiadiazole-2-carboxylate

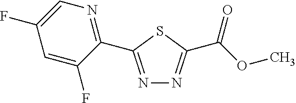 [SMILES: COC(=O)c1nnc(-c2ncc(F)cc2F)s1]

A solution of methyl 2-[2-(3,5-difluoropyridine-2-carbonyl)hydrazino]-2-oxo-acetate (0.5 g, 2 mmol) and phosphorus pentasulfide (0.1 mL, 1 mmol) in toluene (5 mL) was refluxed for 3 hr. The progress of the reaction was monitored by LCMS and upon completion, the reaction mixture was quenched with water and extracted with EtOAc (×3). The combined organic layers were dried over sodium sulphate and concentrated under reduced pressure to obtain the crude product which was purified by silica gel column chromatography (eluting with 0-25% EtOAc in Cyclohexane) to obtain methyl 5-(3,5-difluoro-2-pyridyl)-1,3,4-thiadiazole-2-carboxylate. LCMS (Method B): retention time 1.02 min, m/z 258 (M+1)

### Step 8: Preparation of N-[2-(6-chloro-2-pyridyl)-2-(1-methylpyrazol-4-yl)propyl]-5-(3,5-difluoro-2-pyridyl)-1,3,4-thiadiazole-2-carboxamide (compound X.15, Table P)

To a solution of methyl 5-(3,5-difluoro-2-pyridyl)-1,3,4-thiadiazole-2-carboxylate (0.05 g, 0.19 mmol) in toluene (1 mL), was added 2-(6-chloro-2-pyridyl)-2-(1-methylpyrazol-4-yl)propan-1-amine (as prepared in Example P3, step 3) (0.058 g, 0.23 mmol), at 0° C. To this was added a solution of trimethylaluminum solution (2.0 mol/L) in toluene (0.29 mL, 0.58 mmol) and the reaction mixture then heated to 70° C. and stirred at this temperature. After the completion of the reaction (LCMS analysis) the reaction mixture was quenched slowly in ice cooled brine solution and the brine solution then extracted with EtOAc (×3). The combined organic layers were dried over sodium sulphate and concentrated under reduced pressure. The crude product was adsorbed onto silica and purified by normal phase column chromatography using (0-60% EtOAc in cyclohexane) to obtain the pure compound N-[2-(6-chloro-2-pyridyl)-2-(1-methylpyrazol-4-yl)propyl]-5-(3,5-difluoro-2-pyridyl)-1,3,4-thiadiazole-2-carboxamide as a white solid. LCMS (Method B): retention time 1.15 min, m/z 476 (M+H); ¹H NMR (400 MHz, CDCl₃) δ ppm 8.46 (d, J=2.25 Hz, 1H), 8.30 (br t, J=6.32 Hz, 1H), 7.59 (t, J=7.82 Hz, 1H), 7.45 (ddd, J=9.72, 7.72, 2.31 Hz, 1H), 7.33 (s, 1H), 7.29-7.21 (m, 2H), 7.14-7.10 (m, 1H), 4.18-4.03 (m, 2H), 3.87 (s, 3H), 1.74 (s, 3H)

### Example P4: Preparation of N-[2-(6-cyano-2-pyridyl)-2-(1-methylpyrazol-4-yl)propyl]-5-(3,5-difluoro-2-pyridyl)-1,3,4-thiadiazole-2-carboxamide (Compound X.14. Table P)

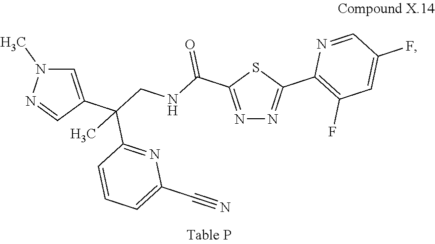 [SMILES: Cn1cc(C(C)(CNC(=O)c2nnc(-c3ncc(F)cc3F)s2)c2cccc(C#N)n2)cn1]

A solution of N-[2-(6-chloro-2-pyridyl)-2-(1-methylpyrazol-4-yl)propyl]-5-(3,5-difluoro-2-pyridyl)-1,3,4-thiadiazole-2-carboxamide (0.1 g, 0.21 mmol, prepared as described in example P3, vide supra) and zinc cyanide (0.05 g, 0.42 mmol) in N,N-Dimethylformamide (1.05 mL) was de-gassed under nitrogen for 10 min. To this solution was added tetrakis(triphenylphosphine)palladium(0) (24.5 mg, 0.021 mmol) and the resultant pale brown suspension was stirred in microwave at 120° C. for 3 hr. The progress of the reaction was monitored by LCMS, and upon completion, the reaction mixture was cooled and diluted with ice cold water (20 mL). The mixture was extracted with EtOAc (×3) and the combined organic layers were washed with brine, dried over sodium sulphate, and concentrated under reduced pressure. The crude product was absorbed on silica gel and purified by combiflash eluting with 0-80% EtOAc in cyclohexane to yield the product as a yellow gummy solid. The product was further purified by reverse phase column chromatography by using 0-70% acetonitrile in water to afford N-[2-(6-cyano-2-pyridyl)-2-(1-methylpyrazol-4-yl)propyl]-5-(3,5-difluoro-2-pyridyl)-1,3,4-thiadiazole-2-carboxamide as yellow gummy mass. LCMS (Method B): retention time 1.04 min, m/z 467 (M+H); ¹H NMR (400 MHz, CDCl₃) δ ppm 8.46 (d, J=2.25 Hz, 1H), 8.00 (brt, J=6.44 Hz, 1H), 7.77 (t, J=7.85 Hz, 1H), 7.63 (d, J=7.67 Hz, 1H), 7.45-7.42 (m, 2H), 7.30-7.31 (m, 1H), 7.26 (d, J=0.75 Hz, 1H), 4.25-4.05 (m, 2H), 3.88 (s, 3H), 1.76 (s, 3H)

### Example P5: Preparation of N-[2-(6-chloro-2-pyridyl)-2-(1-methylpyrazol-4-yl)propyl]-5-(3,5-difluoro-2-pyridyl)isoxazole-3-carboxamide (Compound X.11, Table P)

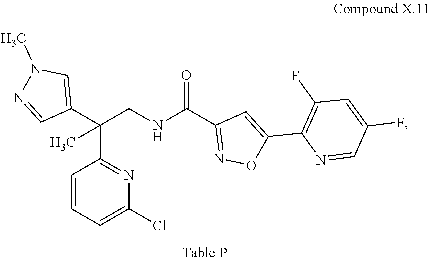 [SMILES: Cn1cc(C(C)(CNC(=O)c2cc(-c3ncc(F)cc3F)on2)c2cccc(Cl)n2)cn1]

### Step 1: Preparation of 3,5-difluoro-N-methoxy-N-methyl-pyridine-2-carboxamide

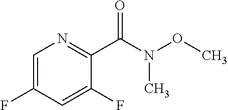

To a suspension of 3,5-difluoropyridine-2-carboxylic acid (10 g, 62.8 mmol) and N,O-dimethylhydroxylamine hydrochloride (6.56 g, 66.0 mmol) in EtOAc (250 mL) was added 1-propanephosphonic anhydride (74.8 mL, 125 mmol) followed by the N,N-diisopropylethylamine (33.0 mL, 188 mmol), at rt. The resulting reaction mixture was stirred at rt for 18 hr where TLC analysis showed reaction completion. The reaction mixture was diluted with water (100 mL) and extracted with EtOAc (×3). The combined organic layers were washed with brine, dried over sodium sulphate, and concentrated under reduced pressure. The crude product was purified by combi flash chromatography to afford 3,5-difluoro-N-methoxy-N-methyl-pyridine-2-carboxamide as a brown liquid. LCMS (Method B): retention time 0.60 min, m/z 203 (M+H)

### Step 2: Preparation of 1-(3,5-difluoro-2-pyridyl)ethenone

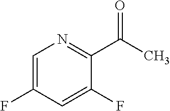

A solution of methyl magnesium bromide (2M in THF, 33 mL, 98.9 mmol) was added dropwise to 3,5-difluoro-N-methoxy-N-methyl-pyridine-2-carboxamide (10 g, 49.4 mmol) in dry THE (150 mL) at −15° C. under nitrogen. The resulting pale brown suspension was allowed to warm to rt and stirred for 1 hr where LCMS and TLC analysis showed reaction completion. The reaction mixture was slowly quenched with concentrated hydrochloric acid (10.7 mL, 118 mmol) at 0° C. The mixture was extracted with EtOAc (×3) and the organic layers combined, washed successively with water and brine, dried over anhydrous sodium sulphate, filtered, and concentrated under reduced pressure to give 1-(3,5-difluoro-2-pyridyl) ethanone as a brown liquid which was used without further purification. LCMS (Method A): retention time 0.94 min, m/z 158.0 (M+H)

### Step 3: Preparation of ethyl 5-(3,5-difluoro-2-pyridyl)-5-hydroxy-4H-isoxazole-3-carboxylate

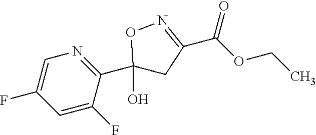 [SMILES: CCOC(=O)C1=NOC(O)(c2ncc(F)cc2F)C1]

A solution of 1-(3,5-difluoro-2-pyridyl)ethanone (2.00 g, 12.09 mmol) and diethyl oxalate (9.22 mL, 66.5 mmol) in toluene (40 mL) was treated with potassium tert-butoxide (1.39 g, 12.1 mmol) at −78° C. The cooling bath was removed, and the resulting suspension was allowed to warm to rt and stirred for 20 min. The reaction mixture was then cooled to −5-0° C. and treated with hydroxylamine hydrochloride (1.71 g, 24.2 mmol) and acetic acid (2.16 mL, 36.2 mmol) and stirred for 12 hr at rt. The reaction mixture was poured into water (50 mL) and extracted with EtOAc (×3). The combined organic layers were washed with brine, dried over anhydrous sodium sulfate, filtered, and concentrated under reduced pressure. The resulting crude residue containing ethyl 5-(3,5-difluoro-2-pyridyl)-5-hydroxy-4H-isoxazole-3-carboxylate was used as such for the next step. LCMS (Method B): retention time 1.63 min, m/z 272 (M+)

### Step 4: Preparation of ethyl 5-(3,5-difluoro-2-pyridyl)isoxazole-3-carboxylate

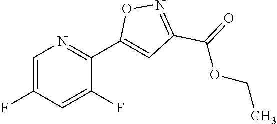 [SMILES: CCOC(=O)c1cc(-c2ncc(F)cc2F)on1]

To a stirred solution of ethyl 5-(3,5-difluoro-2-pyridyl)-5-hydroxy-4H-isoxazole-3-carboxylate (12.8 g, 47.0 mmol) in toluene (256 mL) was added p-toluenesulfonic acid (8.52 g, 47.0 mmol) at rt. The reaction mixture was then heated to 90° C. and stirred for 16 hr. After completion of reaction, the mixture was cooled to 0° C. and slowly quenched with saturated aqueous sodium bicarbonate solution. The mixture was extracted with EtOAc (×3) and the combined organic layers were washed with brine, dried over anhydrous sodium sulfate, filtered, and concentrated under reduced pressure. The crude product was purified by reverse phase column and again purified by normal phase to get ethyl 5-(3,5-difluoro-2-pyridyl)isoxazole-3-carboxylate as a white solid. LCMS (Method B): retention time 1.88 min, m/z 255.0 (M+H)

### Step 5: Preparation of 5-(3,5-difluoro-2-pyridyl)isoxazole-3-carboxylic acid

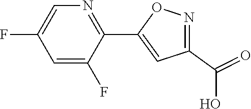 [SMILES: O=C(O)c1cc(-c2ncc(F)cc2F)on1]

A solution of ethyl 5-(3,5-difluoro-2-pyridyl)isoxazole-3-carboxylate (4.95 g, 18.5 mmol) in THF (20 mL) and water (5 mL) was treated with lithium hydroxide hydrate (0.88 g, 37.0 mmol) and stirred at rt for 2 hr. After completion of the reaction, the reaction mixture was diluted with water and extracted with TBME. The aqueous layer was then acidified with 2N HCl—. This led to precipitation of a solid, which was filtered over a Buchner funnel and concentrated under reduced pressure to yield 5-(3,5-difluoro-2-pyridyl)isoxazole-3-carboxylic acid as off-white solid. LCMS (Method B): retention time 0.31 min, m/z 227 (M+H)

### Step 6: Preparation of N-[2-(6-chloro-2-pyridyl)-2-(1-methylpyrazol-4-yl)propyl]-5-(3,5-difluoro-2-pyridyl)isoxazole-3-carboxamide (Compound X.11, Table P)

A solution of 2-(6-chloro-2-pyridyl)-2-(1-methylpyrazol-4-yl)propan-1-amine (13.7 g, 53.6 mmol, prepared as described in example P1,vide infra) and 5-(3,5-difluoro-2-pyridyl)isoxazole-3-carboxylic acid (13.5 g, 53.6 mmol) in EtOAc (214 mL) was treated with N,N-diisopropylethylamine (28.1 mL, 161 mmol) and 1-propanephosphonic anhydride (63.9 mL, 107 mmol) at rt. The reaction mixture was stirred at rt monitoring by TLC and LCMS. After reaction completion, the mixture was diluted with cold water and extracted with EtOAc (×3). The combined organic layers were washed with brine (40 mL), dried over sodium sulphate, and concentrated under reduced pressure. The crude product was purified by reverse phase combiflash using 0-70% acetonitrile in water as eluent to yield the product N-[2-(6-chloro-2-pyridyl)-2-(1-methylpyrazol-4-yl)propyl]-5-(3,5-difluoro-2-pyridyl)isoxazole-3-carboxamide as an off white solid. LCMS (Method B): retention time 1.12 min, m/z 459 (M+H); ¹H NMR (400 MHz, CDCl₃) δ ppm 8.52 (d, 1H), 8.01 (t, 1H), 7.61-7.57 (t, 1H), 7.43-7.39 (m, 1H), 7.33 (s, 1H), 7.28-7.22 (m, 3H), 7.12-7.10 (d, 1H), 4.12-4.01 (m, 2H), 3.88 (s, 3H), 1.73 (s, 3H); ¹⁹F NMR (400 MHz, CDCl₃) δ ppm −114.22 (s, 1F) −118.96 (s, 1F)

### Example P6: Preparation of N-[2-(6-chloro-2-pyridyl)-2-(1-methylpyrazol-4-yl)propyl]-5-(2,6-difluoro-3-pyridyl)-1,3,4-thiadiazole-2-carboxamide (Compound X.16. Table P)

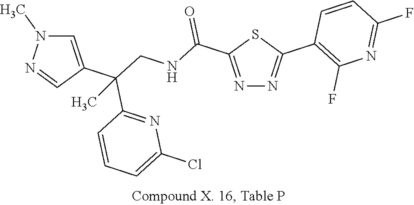 [SMILES: Cn1cc(C(C)(CNC(=O)c2nnc(-c3ccc(F)nc3F)s2)c2cccc(Cl)n2)cn1]

### Step 1: Preparation of 2,6-difluoropyridine-3-carbonyl chloride

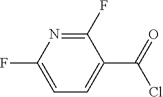 [SMILES: O=C(Cl)c1ccc(F)nc1F]

A To a solution of 2,6-difluoropyridine-3-carboxylic acid (10.0 g, 59.7 mmol) in EtOAc (200 mL) was added dropwise oxalyl chloride (7.89 mL, 89.6 mmol). Catalytic dimethyl formamide (0.46 mL, 5.9 mmol) was added dropwise and the resulting reaction mixture was allowed to stir at rt for 1 hr. After the completion of the reaction, the reaction mixture was concentrated under reduced pressure to obtain the crude title product, which was used as such without any further purification.

### Step 2: Preparation of tert-butyl N-[(2,6-difluoropyridine-3-carbonyl)amino]carbamate

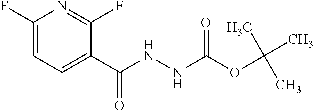 [SMILES: CC(C)(C)OC(=O)NNC(=O)c1ccc(F)nc1F]

A solution of 2,6-difluoropyridine-3-carbonyl chloride (11 g, 58.8 mmol) in EtOAc (110 mL) was added dropwise under stirring to a solution of tert-butyl N-aminocarbamate (7.78 g, 58.8 mmol) in EtOAc (80 mL) at rt. The resulting reaction mixture was allowed to stir at rt and then diluted with water and extracted with EtOAc (×3). The combined organic layers were washed with brine, dried over sodium sulphate, and concentrated under reduced pressure to obtain the crude title compound as a white solid which was used as such for the next step without any further purification. LCMS (Method B): retention time 0.99 min, m/z 174 (M-100)

### Step 3: Preparation of 2,6-difluoropyridine-3-carbohydrazide

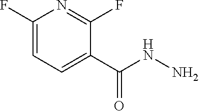 [SMILES: NNC(=O)c1ccc(F)nc1F]

A sample of tert-butyl N-[(2,6-difluoropyridine-3-carbonyl)amino]carbamate (8.9 g, 31 mmol) was dissolved in 4M hydrochloric acid in dioxane (77 mL, 310 mmol) under stirring and the resulting reaction mixture was allowed to stir at rt for 12 hr. After the completion of the reaction, the reaction mixture was diluted with water and extracted with EtOAc (×3). The combined organic layers were washed with brine, dried over sodium sulphate, and concentrated under reduced pressure to obtain the title compound as a pale-yellow solid which was used as such for the next step without any further purification. LCMS (Method B): retention time 0.19 min, m/z 174 (M+H)

### Step 4: Preparation of methyl 2-[2-(2,6-difluoropyridine-3-carbonyl)hydrazino]-2-oxo-acetate

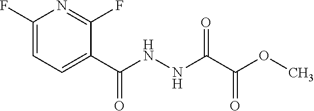 [SMILES: COC(=O)C(=O)NNC(=O)c1ccc(F)nc1F]

To a solution of 2,6-difluoropyridine-3-carbohydrazide (3.45 g, 18.9 mmol) and triethylamine (5.3 mL, 37.9 mmol) in DCM (35 mL) was added dropwise methyl oxalyl chloride (1.82 mL, 18.9 mmol) at 0° C. The progress of the reaction was monitored by LCMS and upon completion, the reaction mixture was quenched with 150 mL sat. sodium bicarbonate solution and extracted with EtOAc (2×100 mL). The combined organic layers were washed successively with water and brine, dried over sodium sulphate, and concentrated under reduced pressure to obtain the title compound as a pale-yellow solid which was used as such for the next step without further purification. LCMS (Method C): retention time 0.30 min, m/z 260 (M+H)

### Step 5: Preparation of methyl 5-(2,6-difluoro-3-pyridyl)-1,3,4-thiadiazole-2-carboxylate

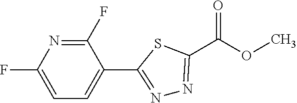 [SMILES: COC(=O)c1nnc(-c2ccc(F)nc2F)s1]

To a solution of methyl 2-[2-(2,6-difluoropyridine-3-carbonyl)hydrazino]-2-oxo-acetate (0.75 g, 2.31 mmol) in THE (15 mL) was added Lawesson's reagent (1.15 g, 2.78 mmol) and the reaction mixture was refluxed for 3 hr. The progress of the reaction was monitored by LCMS and upon completion, the reaction mixture was diluted with water and extracted with EtOAc. The organic layer was washed with brine, dried over sodium sulphate, and concentrated under reduced pressure. The resulting crude product was purified by silica gel chromatography (eluting with 0-30% EtOAc in cyclohexane) to afford methyl 5-(2,6-difluoro-3-pyridyl)-1,3,4-thiadiazole-2-carboxylate as a pale-yellow solid. LCMS (Method A): retention time 1.07 min, 258 (M+H)

### Step 6: Preparation of N-[2-(6-chloro-2-pyridyl)-2-(1-methylpyrazol-4-yl)propyl]-5-(2,6-difluoro-3-pyridyl)-1,3,4-thiadiazole-2-carboxamide (Compound X.16, Table P)

To a solution of methyl 5-(2,6-difluoro-3-pyridyl)-1,3,4-thiadiazole-2-carboxylate (0.15 g, 0.55 mmol) and 2-(6-chloro-2-pyridyl)-2-(1-methylpyrazol-4-yl)propan-1-amine (as prepared in Example P3, step 3) (0.17 g, 0.66 mmol, prepared as described in example P1 vide supra) in toluene (3 mL) was added dropwise trimethylaluminum solution (2.0 mol/L) in toluene (0.83 mL, 1.66 mmol) at 0° C., The resulting reaction mixture was stirred at 70° C. for 2 hr. After the completion of the reaction, the reaction mixture was quenched slowly with ice cooled brine solution, and then was extracted with EtOAc (×3). The combined organic layers were dried over sodium sulphate and concentrated under reduced pressure. The crude product was adsorbed onto silica and purified by normal phase column chromatography eluting with 0-50% EtOAc in cyclohexane to yield N-[2-(6-chloro-2-pyridyl)-2-(1-methylpyrazol-4-yl)propyl]-5-(2,6-difluoro-3-pyridyl)-1,3,4-thiadiazole-2-carboxamide as a pale brown solid. LCMS (Method B): retention time 1.17 min, m/z 476 (M+H); ¹H NMR (400 MHz, CDCl₃) δ ppm 8.92-9.00 (m, 1H), 8.41 (br t, J=6.30 Hz, 1H), 7.60 (t, J=7.65 Hz, 1H), 7.32 (s, 1H), 7.23-7.30 (m, 2H), 7.12 (d, J=7.70 Hz, 1H), 7.08 (dd, J=8.38, 2.63 Hz, 1H), 4.04-4.18 (m, 2H), 3.88 (s, 3H), 1.74 (s, 3H)

### Example P7: Preparation of N-[2-(6-chloro-2-pyridyl)-2-(1-methylpyrazol-4-yl)propyl]-3-(2,4-difluorophenyl)-1,2,4-oxadiazole-5-carboxamide (Compound X.12 Table P)

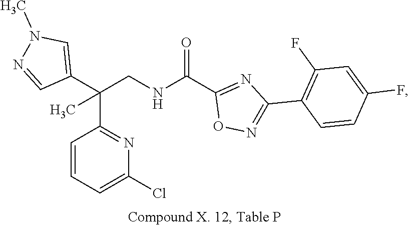 [SMILES: Cn1cc(C(C)(CNC(=O)c2nc(-c3ccc(F)cc3F)no2)c2cccc(Cl)n2)cn1]

### Step 1: Preparation of 2,4-difluoro-N′-hydroxy-benzamidine

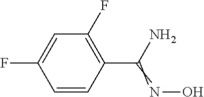 [SMILES: NC(=NO)c1ccc(F)cc1F]

To a solution of 2,4-difluorobenzonitrile ([CAS: 3939-09-1], 5 g, 35.94 mmol) in ethanol (143 mL) was added hydroxylamine (50% solution in water, 4.40 mL, 71.89 mmol). The reaction mixture was stirred for 1 hour at 50° C. Ethanol was evaporated, the residue was dissolved in EtOAc and washed with brine. The organic layer was dried over Na₂SO₄, filtered and concentrated under reduced pressure to afford 2,4-difluoro-N′-hydroxy-benzamidine. LC-MS (Method B): retention time 0.22 min, m/z 173 [M+H+]; ¹H NMR (400 MHz, CDCl₃) δ ppm: 7.70 (td, J=8.63, 6.50 Hz, 1H) 6.85-6.99 (m, 2H) 5.13 (br s, 2H)

### Step 2: Preparation of methyl 3-(2,4-difluorophenyl)-1,2,4-oxadiazole-5-carboxylate

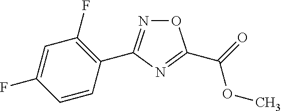 [SMILES: COC(=O)c1nc(-c2ccc(F)cc2F)no1]

N,N-Diisopropylethylamine (19 mL, 110 mL) was added dropwise to a stirred suspension of 2,4-difluoro-N′-hydroxy-benzamidine (6.2 g, 36 mmol) in acetonitrile (160 mL), at rt, under nitrogen. The mixture was cooled to 0° C. and methyl 2-chloro-2-oxo-acetate (5.0 mL, 54 mmol) added dropwise over 10 min. After stirring at 0° C. for 10 min, the mixture was warmed to rt for 10 min and then to 80° C. and stirred for 2 hr. After completion, the reaction mixture was cooled down to rt, quenched with sat. NH₄Cl, and the aqueous phase was extracted with EtOAc (×3). The organic phases were combined, washed with sat NaHCO₃ and brine, dried over Na₂SO₄, filtered and concentrated under reduced pressure. The crude product was adsorbed on silica and purified on combiflash using a silica gel column and eluting with EtOAc and cyclohexane to obtain methyl 3-(2,4-difluorophenyl)-1,2,4-oxadiazole-5-carboxylate. LC-MS (Method B): retention time 1.04 min, m/z desired mass not detected; ¹H NMR (400 MHz, CDCl₃) δ ppm: 8.13-8.20 (m, 1H) 7.02-7.10 (m, 2H) 4.14 (s, 3H)

### Step 3: Preparation of N-[2-(6-chloro-2-pyridyl)-2-(1-methylpyrazol-4-yl)propyl]-3-(2,4-difluorophenyl)-1,2,4-oxadiazole-5-carboxamide (Compound X.12, Table P)

In a 250 ml single neck RBF, methyl 3-(2,4-difluorophenyl)-1,2,4-oxadiazole-5-carboxylate (4 g, 16.656 mmol) and 2-(6-chloro-2-pyridyl)-2-(1-methylpyrazol-4-yl)propan-1-amine (as prepared in Example P3, step 3) (5.0 g, 19.987 mmol) was taken in dry tetrahydrofuran (40 mL) and to this mixture, magnesium chloride (5.97 g, 99.933 mmol) was added under nitrogen. The reaction mixture was stirred at rt under inert atmosphere for 3 hr. After the completion of the reaction, the reaction mixture was quenched with water (60 ml). The brine solution was extracted with EtOAc (2×80 mL). The combined organic layer was dried over sodium sulphate and concentrated under reduced pressure to obtain the crude. The crude was adsorbed over silica and purified by normal phase column chromatography using (0-50% EA in cyclohexane) to afford N-[2-(6-chloro-2-pyridyl)-2-(1-methylpyrazol-4-yl)propyl]-3-(2,4-difluorophenyl)-1,2,4-oxadiazole-5-carboxamide. LC-MS (Method B): retention time 1.20 min, m/z 459 (M+H); ¹H NMR (400 MHz, DMSO-d6) δ ppm 9.11 (t, J=6.37 Hz, 1H) 8.14-8.07 (m, 1H) 7.79 (t, J=7.89 Hz, 1H) 7.63 (s, 1H) 7.60 (br dd, J=11.00, 2.32 Hz, 1H) 7.41-7.34 (m, 3H) 7.27 (d, J=7.70 Hz, 1H) 4.05 (dd, J=13.27, 6.17 Hz, 1H) 3.87 (dd, J=13.33, 6.60 Hz, 1H) 3.78 (s, 3H) 1.66 (s, 3H)

Where necessary, enantiomerically pure final compounds may be obtained from racemic materials as appropriate via standard physical separation techniques, such as reverse phase chiral chromatography, or through stereoselective synthetic techniques, (e.g., by using chiral starting materials).

Examples of synthesized compounds of formula (I) (component A) are shown in Table P.

TABLE P
Synthesized compounds and Spectral and Physical Chemical Data for compounds X.01 to X.16
according to formula (I):

| | | | RT | [M + H] | |
|---|---|---|---|---|---|
| Entry | IUPAC name | Structure | (min) | (measured) | Method |
| X.01 | N-[2-(6-chloro-2-pyridyl)-2-(1- methylpyrazol-4-yl)propyl]-5-(2,4- difluorophenyl)isoxazole-3- carboxamide | 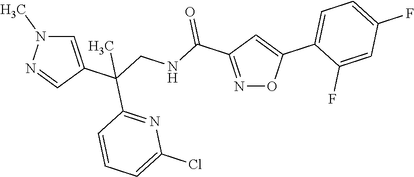 [SMILES: Cn1cc(C(C)(CNC(=O)c2cc(-c3ccc(F)cc3F)on2)c2cccc(Cl)n2)cn1] | 1.08 | 458 | A |
| X.02 | N-[2-(6-cyano-2-pyridyl)-2-(1- methylpyrazol-4-yl)propyl]-5-(2,4- difluorophenyl)isoxazole-3- carboxamide | 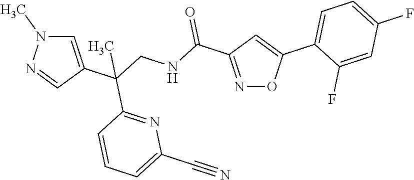 [SMILES: Cn1cc(C(C)(CNC(=O)c2cc(-c3ccc(F)cc3F)on2)c2cccc(C#N)n2)cn1] | 0.99 | 449 | A |
| X.03 | 5-(2,4-difluorophenyl)-N-[2-(1- methylpyrazol-4-yl)-2-(2- pyridyl)propyl]isoxazole-3- carboxamide | 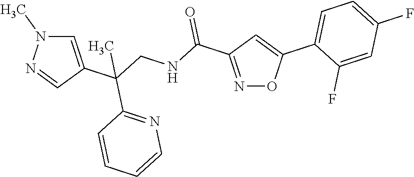 [SMILES: Cn1cc(C(C)(CNC(=O)c2cc(-c3ccc(F)cc3F)on2)c2ccccn2)cn1] | 0.96 | 424 | A |
| X.04 | N-[(2S)-2-(6-chloro-4-methoxy-2- pyridyl)-2-(1-methylpyrazol-4- yl)propyl]-3-(2,4- difluorophenyl)isoxazole-5- carboxamide | 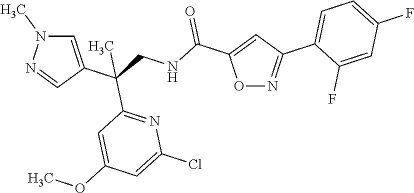 [SMILES: COc1cc(Cl)nc([C@](C)(CNC(=O)c2cc(-c3ccc(F)cc3F)no2)c2cnn(C)c2)c1] | 1.09 | 488 | A |
| X.05 | 5-(2,4-difluorophenyl)-N-[2-[6- [(E)-N-methoxy-C-methyl- carbonimidoyl]-2-pyridyl]-2-(1- methylpyrazol-4- yl)propyl]isoxazole-3- carboxamide | 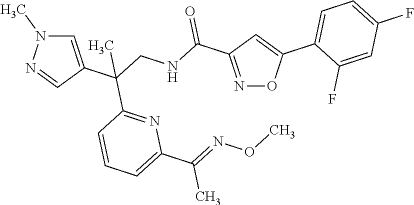 [SMILES: CO/N=C(\C)c1cccc(C(C)(CNC(=O)c2cc(-c3ccc(F)cc3F)on2)c2cnn(C)c2)n1] | 1.15 | 495 | A |
| X.06 | N-[2-(5-chloro-2-pyridyl)-2-(1- methylpyrazol-4-yl)propyl]-5-(2,4- difluorophenyl)-1,3,4-thiadiazole- 2-carboxamide | 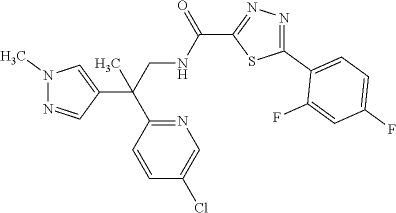 [SMILES: Cn1cc(C(C)(CNC(=O)c2nnc(-c3ccc(F)cc3F)s2)c2ccc(Cl)cn2)cn1] | 1.08 | 475 | A |
| X.07 | N-[(2R)-2-(6-chloro-4-methoxy-2- pyridyl)-2-(1-methylpyrazol-4- yl)propyl]-3-(2,4- difluorophenyl)isoxazole-5- carboxamide | 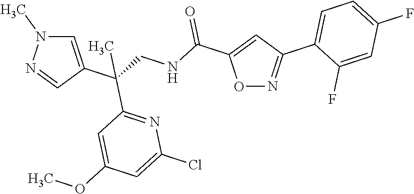 [SMILES: COc1cc(Cl)nc([C@@](C)(CNC(=O)c2cc(-c3ccc(F)cc3F)no2)c2cnn(C)c2)c1] | 1.09 | 488 | A |
| X.08 | 5-(2,4-difluorophenyl)-N-[2-(1- methylpyrazol-4-yl)-2-(5-methyl-2- pyridyl)propyl]isoxazole-3- carboxamide | 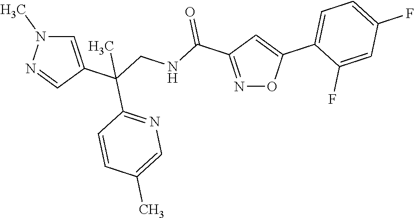 [SMILES: Cc1ccc(C(C)(CNC(=O)c2cc(-c3ccc(F)cc3F)on2)c2cnn(C)c2)nc1] | 0.98 | 438 | A |
| X.09 | N-[2-(6-chloro-2-pyridyl)-2-(1- methylpyrazol-4-yl)propyl]-5-(2,4- dichlorophenyl)isoxazole-3- carboxamide | 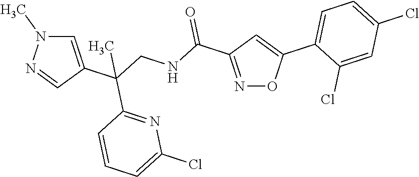 [SMILES: Cn1cc(C(C)(CNC(=O)c2cc(-c3ccc(Cl)cc3Cl)on2)c2cccc(Cl)n2)cn1] | 1.17 | 490 | A |
| X.10 | methyl 6-[2-[[5-(2,4- difluorophenyl)isoxazole-3- carbonyl]amino]-1-methyl-1-(1- methylpyrazol-4-yl)ethyl]pyridine- 3-carboxylate | 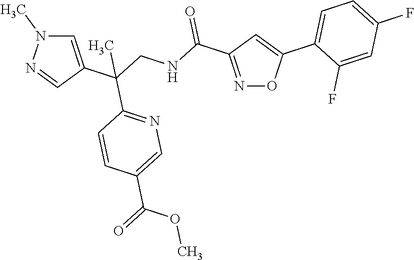 [SMILES: COC(=O)c1ccc(C(C)(CNC(=O)c2cc(-c3ccc(F)cc3F)on2)c2cnn(C)c2)nc1] | 1.02 | 482 | A |
| X.11 | N-[2-(6-chloro-2-pyridyl)-2-(1- methylpyrazol-4-yl)propyl]-5-(3,5- difluoro-2-pyridyl)isoxazole-3- carboxamide | 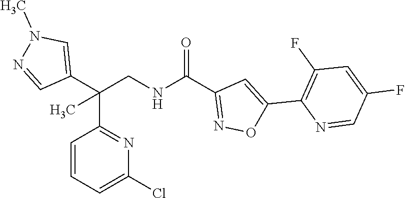 [SMILES: Cn1cc(C(C)(CNC(=O)c2cc(-c3ncc(F)cc3F)on2)c2cccc(Cl)n2)cn1] | 0.96 | 459 | A |
| X.12 | N-[2-(6-chloro-2-pyridyl)-2-(1- methylpyrazol-4-yl)propyl]-3-(2,4- difluorophenyl)-1,2,4-oxadiazole- 5-carboxamide | 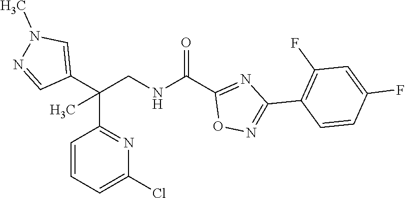 [SMILES: Cn1cc(C(C)(CNC(=O)c2nc(-c3ccc(F)cc3F)no2)c2cccc(Cl)n2)cn1] | 1.53 | 459 | C |
| X.13 | N-[2-(6-chloro-2-pyridyl)-2-(1- methylpyrazol-4-yl)propyl]-5-(2,4- difluorophenyl)-1,3,4-thiadiazole- 2-carboxamide | 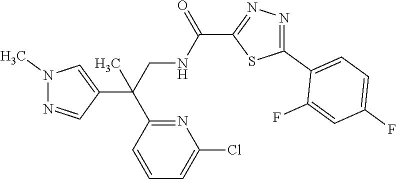 [SMILES: Cn1cc(C(C)(CNC(=O)c2nnc(-c3ccc(F)cc3F)s2)c2cccc(Cl)n2)cn1] | 1.08 | 475 | A |
| X.14 | N-[2-(6-cyano-2-pyridyl)-2-(1- methylpyrazol-4-yl)propyl]-5-(3,5- difluoro-2-pyridyl)-1,3,4- thiadiazole-2-carboxamide | 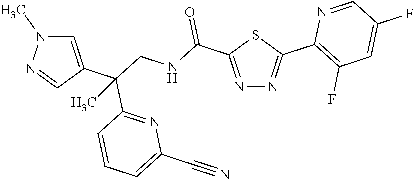 [SMILES: Cn1cc(C(C)(CNC(=O)c2nnc(-c3ncc(F)cc3F)s2)c2cccc(C#N)n2)cn1] | 1.01 | 467 | B |
| X.15 | N-[2-(6-chloro-2-pyridyl)-2-(1- methylpyrazol-4-yl)propyl]-5-(3,5- difluoro-2-pyridyl)-1,3,4- thiadiazole-2-carboxamide | 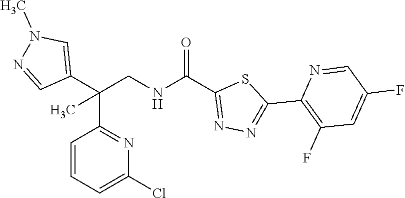 [SMILES: Cn1cc(C(C)(CNC(=O)c2nnc(-c3ncc(F)cc3F)s2)c2cccc(Cl)n2)cn1] | 1.16 | 476 | B |
| X.16 | N-[2-(6-chloro-2-pyridyl)-2-(1- methylpyrazol-4-yl)propyl]-5-(2,6- difluoro-3-pyridyl)-1,3,4- thiadiazole-2-carboxamide | 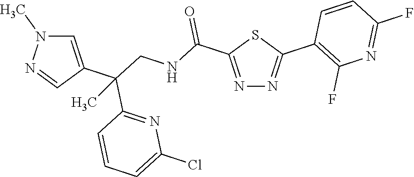 [SMILES: Cn1cc(C(C)(CNC(=O)c2nnc(-c3ccc(F)nc3F)s2)c2cccc(Cl)n2)cn1] | 1.17 | 476 | B |

### BIOLOGICAL EXAMPLES FOR COMPONENT A (SEE TABLE P)

### Example B1: Alternaria solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% rh under a light regime of 12/12 h (light/dark) in a climate cabinet and the activity of a compound is assessed as percent disease control compared to untreated when an appropriate level of disease damage appears on untreated check disk leaf disks (5-7 days after application).

The following compounds gave at least 80% control of Alternaria solani at 200 ppm when compared to untreated control under the same conditions, which showed extensive disease development: X.01, X.02, X.03, X.05, X.08, X.12, X.13

### Example B2: Botryotinia fuckeliana (Botrytis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Botryotinia fuckeliana at 20 ppm when compared to untreated control under the same conditions, which showed extensive disease development: X.01, X.02, X.03, X.04, X.05, X.07, X.08, X.10, X.11, X.12, X.13, X.14, X.15, X.16

### Example B3: Cercospora kikuchii (Leaf Blight of Soybean)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° and the inhibition of growth is determined photometrically 3-4 days after application. The following compounds gave at least 80% control of Botryotinia fuckeliana at 20 ppm when compared to untreated control under the same conditions, which showed extensive disease development: X.01, X.02

### Example B4: Cercospora Sojina (Frogeye Leaf Spot of Soybean)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 3-4 days at 620 nm.

The following compounds gave at least 80% control of Cercospora sojina at 20 ppm when compared to untreated control under the same conditions, which showed extensive disease development: X.01, X.02

### Example B5: Glomerella Lagenarium (Colletotrichum Lagenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3-4 days after application.

The following compounds gave at least 80% control of Glomerella lagenarium at 20 ppm when compared to untreated control under the same conditions, which showed extensive disease development: X.01, X.02, X.03, X.05, X.06, X.07, X.08, X.09, X.10, X.11, X.12, X.13, X.14, X.15, X.16

### Example B6: Corynespora cassiicola (Target Leaf Spot of Tomato)

Conidia of the fungus from cryogenic storage were directly mixed into nutrient broth (PDB potato dextrose broth). A DMSO solution of the test compounds was placed into a microtiter plate (96-well format) and the nutrient broth containing the fungal spores was added to it. The test plates were incubated at 24° C. and the inhibition of growth was determined photometrically after 3-4 days at 620 nm.

The following compounds gave at least 80% control of Corynespora cassiicola at 20 ppm when compared to untreated control under the same conditions, which showed extensive disease development: X.01, X.02

### Example B7: Blumeria Graminis f. Sp. Tritici (Erysiphe graminis f. Sp. Tritici)/Wheat/Leaf Disc Preventative (Powdery Mildew on Wheat)

Wheat leaf segments cv. Kanzler are placed on agar in a multiwell plate (24-well format) and sprayed with the formulated test compound diluted in water. The leaf disks are inoculated by shaking powdery mildew infected plants above the test plates 1 day after application. The inoculated leaf disks are incubated at 20° C. and 60% rh under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application).

The following compounds gave at least 80% control of Blumeria graminis f. sp. tritici at 200 ppm when compared to untreated control under the same conditions, which showed extensive disease development: X.01, X.02, X.03, X.05, X.08, X.11, X.12, X.13, X.14, X.15

### Example B8: Fusarium culmorum/Liquid Culture (Head Blight)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Fusarium culmorum at 20 ppm when compared to untreated control under the same conditions, which showed extensive disease development: X.01, X.02, X.05, X.07, X.11, X.12, X.13, X.15

### Example B9: Fusarium culmorum/Wheat/Spikelet Preventative (Head Blight)

Wheat spikelets cv. Monsun are placed on agar in multiwell plates (24-well format) and sprayed with the formulated test compound diluted in water. The spikelets are inoculated with a spore suspension of the fungus 1 day after application. The inoculated spikelets are incubated at 20° C. and 60% rh under a light regime of 72 h semi darkness followed by 12 h light/12 h darkness in a climate chamber and the activity of a compound is assessed as percent disease control compared to untreated when an appropriate level of disease damage appears on untreated check spikelets (6-8 days after application).

The following compounds gave at least 80% control of Fusarium culmorum at 200 ppm when compared to untreated control under the same conditions, which showed extensive disease development: X.01, X.05, X.07, X.15

### Example B10: Gibberella zeae (Fusarium graminearum)/Wheat/Spikelet Preventative (Head Blight)

Wheat spikelets cv. Monsun are placed on agar in multiwell plates (24-well format) and sprayed with the formulated test compound diluted in water. One day after application, the spikelets are inoculated with a spore suspension of the fungus. The inoculated test leaf disks are incubated at 20° C. and 60% rh under a light regime of 72 h semi darkness followed by 12 h light/12 h darkness in a climate chamber, the activity of a compound is assessed as percent disease control compared to untreated when an appropriate level of disease damage appears on untreated check spikelets (6-8 days after application).

The following compounds gave at least 80% control of Gibberella zeae at 200 ppm when compared to untreated control under the same conditions, which showed extensive disease development: X.07, X.11, X.13

### Example B11: Phaeosphaeria Nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phaeosphaeria nodorum at 200 ppm when compared to untreated control under the same conditions, which showed extensive disease development: X.01, X.02, X.03, X.05, X.12, X.13, X.15

### Example B12: Monographella Nivalis (Microdochium Nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Monographella nivalis at 20 ppm when compared to untreated control under the same conditions, which showed extensive disease development: X.01, X.02, X.03, X.04, X.05, X.06, X.07, X.08, X.09, X.10, X.11, X.12, X.13, X.15, X.16

### Example B13: Mycosphaerella arachidis (Cercospora arachidicola)/Liquid Culture (Early Leaf Spot)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella arachidis at 20 ppm when compared to untreated control under the same conditions, which showed extensive disease development: X.01, X.02, X.05, X.06, X.07, X.11, X.12, X.13, X.15, X.16

### Example B14: Puccinia recondita f. Sp. Tritici/Wheat/Leaf Disc Curative (Brown Rust)

Wheat leaf segments cv. Kanzler are placed on agar in multiwell plates (24-well format). The leaf segments are inoculated with a spore suspension of the fungus. Plates are stored in darkness at 19° C. and 75% rh. The formulated test compound diluted in water is applied 1 day after inoculation. The leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (6-8 days after application).

The following compounds gave at least 80% control of Puccinia recondita f. sp. tritici at 200 ppm when compared to untreated control underthe same conditions, which showed extensive disease development: X.08

### Example B15: Puccinia recondita f. Sp. Tritici/Wheat/Leaf Disc Preventative (Brown Rust)

Wheat leaf segments cv. Kanzler are placed on agar in multiwell plates (24-well format) and sprayed with the formulated test compound diluted in water. The leaf disks are inoculated with a spore suspension of the fungus 1 day after application. The inoculated leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (7-9 days after application).

The following compounds gave at least 80% control of Puccinia recondita f. sp. tritici at 200 ppm when compared to untreated control underthe same conditions, which showed extensive disease development: X.01

### Example B16: Magnaporthe grisea (Pyricularia Oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5-7 days after application).

The following compounds gave at least 80% control of Magnaporthe grisea at 200 ppm when compared to untreated control under the same conditions, which showed extensive disease development: X.01, X.02, X.05

### Example B17: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5-7 days after application).

The following compounds gave at least 80% control of Pyrenophora teres at 200 ppm when compared to untreated control under the same conditions, which showed extensive disease development: X.01, X.02, X.03, X.05, X.08, X.12, X.13, X.14, X.15

### Example B18: Sclerotinia sclerotiorum/Liquid Culture (Cottony Rot)

Mycelia fragments of a newly grown liquid culture of the fungus are directly mixed into nutrient broth (PDB potato dextrose broth). After placing a (DMSO) solution of test compound into a microtiter plate (96-well format) the nutrient broth containing the fungal material is added. The test plates are incubated at 24° C. and the inhibition of growth is determined photometrically 3-4 days after application.

The following compounds gave at least 80% control of Sclerotinia sclerotiorum at 20 ppm when compared to untreated control under the same conditions, which showed extensive disease development: X.01, X.05, X.11, X.12, X.13, X.15

### Example B19: Mycosphaerella graminicola (Septoria tritici)/Liquid Culture (Septoria Blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella graminicola at 20 ppm when compared to untreated control under the same conditions, which showed extensive disease development: X.01, X.02, X.03, X.04, X.05, X.06, X.07, X.08, X.09, X.10, X.11, X.12, X.13, X.14, X.15, X.16

### BIOLOGICAL EXAMPLES FOR FUNGICIDAL MIXTURE

### Example M-B1: Botrytis cinerea (Grey Mould)

Conidia of the fungus from cryogenic storage were directly mixed into nutrient broth (Vogel's). After placing a (DMSO) solution of the test compounds into a microtiter plate (96-well format) the nutrient broth containing the fungal spores was added. The test plates were incubated at 24° C. and the inhibition of growth was determined photometrically and visually after 72 hrs.

The following mixture compositions (A:B) at the reported concentration (in ppm) gave at least 70% disease control in the test system.

The term “component A” or “comp. A” refers to the compound of formula (I).

| | | | Conc. |
|---|---|---|---|
| Comp. | | | (ppm) |
| A | Component B | Ratio A:B | (A:B) |
| X.01 | Pydiflumetofen | 1:30 | 0.2:6 |
| X.01 | Pydiflumetofen | 1:3 | 0.2:0.6 |
| X.01 | Pydiflumetofen | 1:100 | 0.06:6 |
| X.01 | Pydiflumetofen | 1:10 | 0.06:0.6 |
| X.01 | Benzovindiflupyr | 1:10 | 0.2:2 |
| X.01 | Benzovindiflupyr | 1:1 | 0.2:0.2 |
| X.01 | Benzovindiflupyr | 1:33.3 | 0.06:2 |
| X.01 | Benzovindiflupyr | 1:3.3 | 0.06:0.2 |
| X.01 | Cyprodinil | 1:100 | 0.2:20 |
| X.01 | Cyprodinil | 2:1 | 0.2:0.1 |
| X.01 | Cyprodinil | 1:333.3 | 0.06:20 |
| X.01 | Cyprodinil | 1:1.6 | 0.06:0.1 |
| X.01 | Fludioxonil | 1:30 | 0.2:6 |
| X.01 | Fludioxonil | 3.3:1 | 0.2:0.06 |
| X.01 | Fludioxonil | 1:100 | 0.06:6 |
| X.01 | Fludioxonil | 1:1 | 0.06:0.06 |
| X.01 | Azoxystrobin | 1:30 | 0.2:6 |
| X.01 | Azoxystrobin | 1:3 | 0.2:0.6 |
| X.01 | Azoxystrobin | 1:100 | 0.06:6 |
| X.01 | Azoxystrobin | 1:10 | 0.06:0.6 |
| X.01 | Difenoconazole | 1:30 | 0.2:6 |
| X.01 | Difenoconazole | 1:3 | 0.2:0.6 |
| X.01 | Difenoconazole | 1:100 | 0.06:6 |
| X.01 | Prothioconazole | 1:3 | 0.2:0.6 |
| X.01 | Prothioconazole | 3.3:1 | 0.2:0.06 |
| X.01 | Mefentrifluconazole | 1:30 | 0.2:6 |
| X.01 | Mefentrifluconazole | 2:1 | 0.2:0.1 |
| X.01 | Mefentrifluconazole | 1:100 | 0.06:6 |
| X.01 | Mefentrifluconazole | 1:1.6 | 0.06:0.1 |
| X.01 | Florylpicoxamid | 1:5 | 0.2:1 |
| X.01 | Florylpicoxamid | 2:1 | 0.2:0.1 |
| X.01 | Florylpicoxamid | 1:16.6 | 0.06:1 |
| X.01 | Florylpicoxamid | 1:1.6 | 0.06:0.1 |
| X.01 | Acibenzolar-S-methyl | 1:100 | 0.2:20 |
| X.01 | Acibenzolar-S-methyl | 1:30 | 0.2:6 |
| X.01 | Acibenzolar-S-methyl | 1:333.3 | 0.06:20 |
| X.01 | Acibenzolar-S-methyl | 1:100 | 0.06:6 |
| X.05 | Pydiflumetofen | 1:30 | 0.2:6 |
| X.05 | Pydiflumetofen | 1:3 | 0.2:0.6 |
| X.05 | Pydiflumetofen | 1:100 | 0.06:6 |
| X.05 | Pydiflumetofen | 1:10 | 0.06:0.6 |
| X.05 | Benzovindiflupyr | 1:10 | 0.2:2 |
| X.05 | Benzovindiflupyr | 1:1 | 0.2:0.2 |
| X.05 | Benzovindiflupyr | 1:33.3 | 0.06:2 |
| X.05 | Benzovindiflupyr | 1:3.3 | 0.06:0.2 |
| X.05 | Cyprodinil | 1:100 | 0.2:20 |
| X.05 | Cyprodinil | 2:1 | 0.2:0.1 |
| X.05 | Cyprodinil | 1:333.3 | 0.06:20 |
| X.05 | Cyprodinil | 1:1.6 | 0.06:0.1 |
| X.05 | Fludioxonil | 1:30 | 0.2:6 |
| X.05 | Fludioxonil | 3.3:1 | 0.2:0.06 |
| X.05 | Fludioxonil | 1:100 | 0.06:6 |
| X.05 | Fludioxonil | 1:1 | 0.06:0.06 |
| X.05 | Azoxystrobin | 1:30 | 0.2:6 |
| X.05 | Azoxystrobin | 1:3 | 0.2:0.6 |
| X.05 | Azoxystrobin | 1:100 | 0.06:6 |
| X.05 | Azoxystrobin | 1:10 | 0.06:0.6 |
| X.05 | Difenoconazole | 1:30 | 0.2:6 |
| X.05 | Difenoconazole | 1:3 | 0.2:0.6 |
| X.05 | Difenoconazole | 1:100 | 0.06:6 |
| X.05 | Prothioconazole | 1:3 | 0.2:0.6 |
| X.05 | Prothioconazole | 3.3:1 | 0.2:0.06 |
| X.05 | Mefentrifluconazole | 1:30 | 0.2:6 |
| X.05 | Mefentrifluconazole | 2:1 | 0.2:0.1 |
| X.05 | Mefentrifluconazole | 1:100 | 0.06:6 |
| X.05 | Mefentrifluconazole | 1:1.6 | 0.06:0.1 |
| X.05 | Florylpicoxamid | 1:5 | 0.2:1 |
| X.05 | Florylpicoxamid | 2:1 | 0.2:0.1 |
| X.05 | Florylpicoxamid | 1:16.6 | 0.06:1 |
| X.05 | Florylpicoxamid | 1:1.6 | 0.06:0.1 |
| X.05 | Acibenzolar-S-methyl | 1:100 | 0.2:20 |
| X.05 | Acibenzolar-S-methyl | 1:30 | 0.2:6 |
| X.05 | Acibenzolar-S-methyl | 1:333.3 | 0.06:20 |
| X.05 | Acibenzolar-S-methyl | 1:100 | 0.06:6 |
| X.03 | Pydiflumetofen | 1:1 | 6:6 |
| X.03 | Pydiflumetofen | 10:1 | 6:0.6 |
| X.03 | Pydiflumetofen | 1:6 | 1:6 |
| X.03 | Pydiflumetofen | 1.6:1 | 1:0.6 |
| X.03 | Benzovindiflupyr | 3:1 | 6:2 |
| X.03 | Benzovindiflupyr | 30:1 | 6:0.2 |
| X.03 | Benzovindiflupyr | 1:2 | 1:2 |
| X.03 | Benzovindiflupyr | 5:1 | 1:0.2 |
| X.03 | Cyprodinil | 1:3.3 | 6:20 |
| X.03 | Cyprodinil | 60:1 | 6:0.1 |
| X.03 | Cyprodinil | 1:20 | 1:20 |
| X.03 | Cyprodinil | 10:1 | 1:0.1 |
| X.03 | Fludioxonil | 1:1 | 6:6 |
| X.03 | Fludioxonil | 100:1 | 6:0.06 |
| X.03 | Fludioxonil | 1:6 | 1:6 |
| X.03 | Fludioxonil | 16.6:1 | 1:0.6 |
| X.03 | Azoxystrobin | 1:1 | 6:6 |
| X.03 | Azoxystrobin | 10:1 | 6:0.6 |
| X.03 | Azoxystrobin | 1:6 | 1:6 |
| X.03 | Azoxystrobin | 1.6:1 | 1:0.6 |
| X.03 | Difenoconazole | 1:1 | 6:6 |
| X.03 | Difenoconazole | 10:1 | 6:0.6 |
| X.03 | Difenoconazole | 1:6 | 1:6 |
| X.03 | Difenoconazole | 1.6:1 | 1:0.6 |
| X.03 | Prothioconazole | 10:1 | 6:0.6 |
| X.03 | Prothioconazole | 100:1 | 6:0.06 |
| X.03 | Mefentrifluconazole | 1:1 | 6:6 |
| X.03 | Mefentrifluconazole | 60:1 | 6:0.1 |
| X.03 | Mefentrifluconazole | 1:6 | 1:6 |
| X.03 | Mefentrifluconazole | 10:1 | 1:0.1 |
| X.03 | Florylpicoxamid | 6:1 | 6:1 |
| X.03 | Florylpicoxamid | 60:1 | 6:0.1 |
| X.03 | Florylpicoxamid | 1:1 | 1:1 |
| X.03 | Florylpicoxamid | 10:1 | 1:0.1 |
| X.03 | Acibenzolar-S-methyl | 1:3.3 | 6:20 |
| X.03 | Acibenzolar-S-methyl | 1:1 | 6:6 |
| X.08 | Pydiflumetofen | 1:1 | 6:6 |
| X.08 | Pydiflumetofen | 10:1 | 6:0.6 |
| X.08 | Pydiflumetofen | 1:6 | 1:6 |
| X.08 | Pydiflumetofen | 1.6:1 | 1:0.6 |
| X.08 | Benzovindiflupyr | 3:1 | 6:2 |
| X.08 | Benzovindiflupyr | 30:1 | 6:0.2 |
| X.08 | Benzovindiflupyr | 1:2 | 1:2 |
| X.08 | Benzovindiflupyr | 5:1 | 1:0.2 |
| X.08 | Cyprodinil | 1:3.3 | 6:20 |
| X.08 | Cyprodinil | 60:1 | 6:0.1 |
| X.08 | Cyprodinil | 1:20 | 1:20 |
| X.08 | Cyprodinil | 10:1 | 1:0.1 |
| X.08 | Fludioxonil | 1:1 | 6:6 |
| X.08 | Fludioxonil | 100:1 | 6:0.06 |
| X.08 | Fludioxonil | 1:6 | 1:6 |
| X.08 | Fludioxonil | 16.6:1 | 1:0.6 |
| X.08 | Azoxystrobin | 10:1 | 6:0.6 |
| X.08 | Azoxystrobin | 1:6 | 1:6 |
| X.08 | Azoxystrobin | 1.6:1 | 1:0.6 |
| X.08 | Difenoconazole | 1:1 | 6:6 |
| X.08 | Difenoconazole | 10:1 | 6:0.6 |
| X.08 | Difenoconazole | 1:6 | 1:6 |
| X.08 | Prothioconazole | 10:1 | 6:0.6 |
| X.08 | Prothioconazole | 100:1 | 6:0.06 |
| X.08 | Mefentrifluconazole | 1:1 | 6:6 |
| X.08 | Mefentrifluconazole | 60:1 | 6:0.1 |
| X.08 | Mefentrifluconazole | 1:6 | 1:6 |
| X.08 | Mefentrifluconazole | 10:1 | 1:0.1 |
| X.08 | Florylpicoxamid | 6:1 | 6:1 |
| X.08 | Florylpicoxamid | 60:1 | 6:0.1 |
| X.08 | Florylpicoxamid | 1:1 | 1:1 |
| X.08 | Acibenzolar-S-methyl | 1:1 | 6:6 |
| X.10 | Pydiflumetofen | 1:1 | 6:6 |
| X.10 | Pydiflumetofen | 10:1 | 6:0.6 |
| X.10 | Pydiflumetofen | 1:6 | 1:6 |
| X.10 | Pydiflumetofen | 1.6:1 | 1:0.6 |
| X.10 | Benzovindiflupyr | 3:1 | 6:2 |
| X.10 | Benzovindiflupyr | 30:1 | 6:0.2 |
| X.10 | Benzovindiflupyr | 1:2 | 1:2 |
| X.10 | Benzovindiflupyr | 5:1 | 1:0.2 |
| X.10 | Cyprodinil | 1:3.3 | 6:20 |
| X.10 | Cyprodinil | 60:1 | 6:0.1 |
| X.10 | Cyprodinil | 1:20 | 1:20 |
| X.10 | Cyprodinil | 10:1 | 1:0.1 |
| X.10 | Fludioxonil | 1:1 | 6:6 |
| X.10 | Fludioxonil | 100:1 | 6:0.06 |
| X.10 | Fludioxonil | 1:6 | 1:6 |
| X.10 | Fludioxonil | 16.6:1 | 1:0.6 |
| X.10 | Azoxystrobin | 1:1 | 6:6 |
| X.10 | Azoxystrobin | 10:1 | 6:0.6 |
| X.10 | Azoxystrobin | 1:6 | 1:6 |
| X.10 | Azoxystrobin | 1.6:1 | 1:0.6 |
| X.10 | Difenoconazole | 1:1 | 6:6 |
| X.10 | Difenoconazole | 10:1 | 6:0.6 |
| X.10 | Difenoconazole | 1:6 | 1:6 |
| X.10 | Mefentrifluconazole | 1:1 | 6:6 |
| X.10 | Mefentrifluconazole | 60:1 | 6:0.1 |
| X.10 | Mefentrifluconazole | 1:6 | 1:6 |
| X.10 | Mefentrifluconazole | 10:1 | 1:0.1 |
| X.10 | Florylpicoxamid | 6:1 | 6:1 |
| X.10 | Florylpicoxamid | 60:1 | 6:0.1 |
| X.10 | Florylpicoxamid | 1:1 | 1:1 |
| X.10 | Florylpicoxamid | 10:1 | 1:0.1 |
| X.02 | Pydiflumetofen | 1:3 | 2:6 |
| X.02 | Pydiflumetofen | 3.3:1 | 2:0.6 |
| X.02 | Pydiflumetofen | 1:10 | 0.6:6 |
| X.02 | Pydiflumetofen | 1:1 | 0.6:0.6 |
| X.02 | Benzovindiflupyr | 1:1 | 2:2 |
| X.02 | Benzovindiflupyr | 10:1 | 2:0.2 |
| X.02 | Benzovindiflupyr | 1:3.3 | 0.6:2 |
| X.02 | Benzovindiflupyr | 3:1 | 0.6:0.2 |
| X.02 | Cyprodinil | 1:10 | 2:20 |
| X.02 | Cyprodinil | 20:1 | 2:0.1 |
| X.02 | Cyprodinil | 1:33.3 | 0.6:20 |
| X.02 | Cyprodinil | 6:1 | 0.6:0.1 |
| X.02 | Fludioxonil | 1:3 | 2:6 |
| X.02 | Fludioxonil | 33.3:1 | 2:0.06 |
| X.02 | Fludioxonil | 1:10 | 0.6:6 |
| X.02 | Fludioxonil | 10:1 | 0.6:0.06 |
| X.02 | Azoxystrobin | 1:3 | 2:6 |
| X.02 | Azoxystrobin | 3.3:1 | 2:0.6 |
| X.02 | Azoxystrobin | 1:10 | 0.6:6 |
| X.02 | Azoxystrobin | 1:1 | 0.6:0.6 |
| X.02 | Difenoconazole | 1:3 | 2:6 |
| X.02 | Difenoconazole | 3.3:1 | 2:0.6 |
| X.02 | Difenoconazole | 1:10 | 0.6:6 |
| X.02 | Difenoconazole | 1:1 | 0.6:0.6 |
| X.02 | Prothioconazole | 3.3:1 | 2:0.6 |
| X.02 | Prothioconazole | 3.3:1 | 2:0.06 |
| X.02 | Prothioconazole | 1:1 | 0.6:0.6 |
| X.02 | Prothioconazole | 10:1 | 0.6:0.06 |
| X.02 | Mefentrifluconazole | 1:3 | 2:6 |
| X.02 | Mefentrifluconazole | 20:1 | 2:0.1 |
| X.02 | Mefentrifluconazole | 1:10 | 0.6:6 |
| X.02 | Mefentrifluconazole | 6:1 | 0.6:0.1 |
| X.02 | Florylpicoxamid | 2:1 | 2:1 |
| X.02 | Florylpicoxamid | 20:1 | 2:0.1 |
| X.02 | Florylpicoxamid | 1:1.6 | 0.6:1 |
| X.02 | Florylpicoxamid | 6:1 | 0.6:0.1 |
| X.02 | Acibenzolar-S-methyl | 1:10 | 2:20 |
| X.02 | Acibenzolar-S-methyl | 1:3 | 2:6 |
| X.02 | Acibenzolar-S-methyl | 1:33.3 | 0.6:20 |
| X.02 | Acibenzolar-S-methyl | 1:10 | 0.6:6 |
| X.04 | Pydiflumetofen | 1:1 | 6:6 |
| X.04 | Pydiflumetofen | 10:1 | 6:0.6 |
| X.04 | Pydiflumetofen | 1:10 | 0.6:6 |
| X.04 | Pydiflumetofen | 1:1 | 0.6:0.6 |
| X.04 | Benzovindiflupyr | 3:1 | 6:2 |
| X.04 | Benzovindiflupyr | 1:3.3 | 0.6:2 |
| X.04 | Benzovindiflupyr | 3:1 | 0.6:0.2 |
| X.04 | Cyprodinil | 1:3.3 | 6:20 |
| X.04 | Cyprodinil | 60:1 | 6:0.1 |
| X.04 | Cyprodinil | 1:33.3 | 0.6:20 |
| X.04 | Cyprodinil | 6:1 | 0.6:0.1 |
| X.04 | Fludioxonil | 1:1 | 6:6 |
| X.04 | Fludioxonil | 100:1 | 6:0.06 |
| X.04 | Fludioxonil | 1:10 | 0.6:6 |
| X.04 | Fludioxonil | 10:1 | 0.6:0.06 |
| X.04 | Azoxystrobin | 1:10 | 0.6:6 |
| X.04 | Azoxystrobin | 1:1 | 0.6:0.6 |
| X.04 | Difenoconazole | 1:1 | 6:6 |
| X.04 | Difenoconazole | 1:10 | 0.6:6 |
| X.04 | Mefentrifluconazole | 1:1 | 6:6 |
| X.04 | Mefentrifluconazole | 1:10 | 0.6:6 |
| X.04 | Mefentrifluconazole | 6:1 | 0.6:0.1 |
| X.04 | Florylpicoxamid | 1:1.6 | 0.6:1 |
| X.04 | Florylpicoxamid | 6:1 | 0.6:1 |
| X.04 | Acibenzolar-S-methyl | 1:10 | 0.6:6 |
| X.06 | Pydiflumetofen | 3.3:1 | 20:6 |
| X.06 | Pydiflumetofen | 33.3:1 | 20:0.6 |
| X.06 | Pydiflumetofen | 1:6 | 1:6 |
| X.06 | Pydiflumetofen | 1.6:1 | 1:0.6 |
| X.06 | Benzovindiflupyr | 10:1 | 20:2 |
| X.06 | Benzovindiflupyr | 100:1 | 20:0.2 |
| X.06 | Benzovindiflupyr | 1:2 | 1:2 |
| X.06 | Benzovindiflupyr | 5:1 | 1:0.2 |
| X.06 | Cyprodinil | 1:1 | 20:20 |
| X.06 | Cyprodinil | 200:1 | 20:0.1 |
| X.06 | Cyprodinil | 1:20 | 1:20 |
| X.06 | Cyprodinil | 10:1 | 1:0.1 |
| X.06 | Fludioxonil | 3.3:1 | 20:6 |
| X.06 | Fludioxonil | 333.3:1 | 20:0.06 |
| X.06 | Fludioxonil | 1:6 | 1:6 |
| X.06 | Fludioxonil | 16.6:1 | 1:0.6 |
| X.06 | Azoxystrobin | 3.3:1 | 20:6 |
| X.06 | Azoxystrobin | 33.3:1 | 20:0.6 |
| X.06 | Azoxystrobin | 1:6 | 1:6 |
| X.06 | Azoxystrobin | 1.6:1 | 1:0.6 |
| X.06 | Difenoconazole | 3.3:1 | 20:6 |
| X.06 | Difenoconazole | 1:6 | 1:6 |
| X.06 | Mefentrifluconazole | 3.3:1 | 20:6 |
| X.06 | Mefentrifluconazole | 200:1 | 20:0.1 |
| X.06 | Mefentrifluconazole | 1:6 | 1:6 |
| X.06 | Mefentrifluconazole | 10:1 | 1:0.1 |
| X.06 | Florylpicoxamid | 20:1 | 20:1 |
| X.06 | Florylpicoxamid | 200:1 | 20:0.1 |
| X.06 | Florylpicoxamid | 1:1 | 1:1 |
| X.07 | Pydiflumetofen | 1:6 | 1:6 |
| X.07 | Pydiflumetofen | 1.6:1 | 1:0.6 |
| X.07 | Pydiflumetofen | 1:60 | 0.1:6 |
| X.07 | Pydiflumetofen | 1:6 | 0.1:0.6 |
| X.07 | Benzovindiflupyr | 1:2 | 1:2 |
| X.07 | Benzovindiflupyr | 5:1 | 1:0.2 |
| X.07 | Benzovindiflupyr | 1:20 | 0.1:2 |
| X.07 | Benzovindiflupyr | 1:2 | 0.1:0.2 |
| X.07 | Cyprodinil | 1:20 | 1:20 |
| X.07 | Cyprodinil | 10:1 | 1:0.1 |
| X.07 | Cyprodinil | 1:200 | 0.1:20 |
| X.07 | Cyprodinil | 1:1 | 0.1:0.1 |
| X.07 | Fludioxonil | 1:6 | 1:6 |
| X.07 | Fludioxonil | 16.6:1 | 1:0.6 |
| X.07 | Fludioxonil | 1:60 | 0.1:6 |
| X.07 | Fludioxonil | 1.6:1 | 0.1:0.06 |
| X.07 | Azoxystrobin | 1:6 | 1:6 |
| X.07 | Azoxystrobin | 1.6:1 | 1:0.6 |
| X.07 | Azoxystrobin | 1:60 | 0.1:6 |
| X.07 | Azoxystrobin | 1:6 | 0.1:0.6 |
| X.07 | Difenoconazole | 1:6 | 1:6 |
| X.07 | Difenoconazole | 1.6:1 | 1:0.6 |
| X.07 | Difenoconazole | 1:60 | 0.1:6 |
| X.07 | Prothioconazole | 16.6:1 | 1:0.6 |
| X.07 | Mefentrifluconazole | 1:6 | 1:6 |
| X.07 | Mefentrifluconazole | 10:1 | 1:0.1 |
| X.07 | Mefentrifluconazole | 1:60 | 0.1:6 |
| X.07 | Mefentrifluconazole | 1:1 | 0.1:0.1 |
| X.07 | Florylpicoxamid | 1:1 | 1:1 |
| X.07 | Florylpicoxamid | 10:1 | 1:0.1 |
| X.07 | Florylpicoxamid | 1:10 | 0.1:1 |
| X.07 | Florylpicoxamid | 1:1 | 0.1:0.1 |
| X.07 | Acibenzolar-S-methyl | 1:20 | 1:20 |
| X.07 | Acibenzolar-S-methyl | 1:6 | 1:6 |
| X.07 | Acibenzolar-S-methyl | 1:200 | 0.1:20 |
| X.07 | Acibenzolar-S-methyl | 1:60 | 0.1:6 |
| X.09 | Pydiflumetofen | 3.3:1 | 20:6 |
| X.09 | Pydiflumetofen | 33.3:1 | 20:0.6 |
| X.09 | Pydiflumetofen | 1:3 | 2:6 |
| X.09 | Pydiflumetofen | 3.3:1 | 2:0.6 |
| X.09 | Benzovindiflupyr | 10:1 | 20:2 |
| X.09 | Benzovindiflupyr | 100:1 | 20:0.2 |
| X.09 | Benzovindiflupyr | 1:1 | 2:2 |
| X.09 | Benzovindiflupyr | 10:1 | 2:0.2 |
| X.09 | Cyprodinil | 1:1 | 20:20 |
| X.09 | Cyprodinil | 200:1 | 20:0.1 |
| X.09 | Cyprodinil | 1:10 | 2:20 |
| X.09 | Cyprodinil | 20:1 | 2:0.1 |
| X.09 | Fludioxonil | 3.3:1 | 20:6 |
| X.09 | Fludioxonil | 333.3:1 | 20:0.06 |
| X.09 | Fludioxonil | 1:3 | 2:6 |
| X.09 | Fludioxonil | 33.3:1 | 2:0.06 |
| X.09 | Azoxystrobin | 3.3:1 | 20:6 |
| X.09 | Azoxystrobin | 33.3:1 | 20:0.6 |
| X.09 | Azoxystrobin | 1:3 | 2:6 |
| X.09 | Azoxystrobin | 3.3:1 | 2:0.6 |
| X.09 | Difenoconazole | 3.3:1 | 20:6 |
| X.09 | Difenoconazole | 1:3 | 2:6 |
| X.09 | Mefentrifluconazole | 3.3:1 | 20:6 |
| X.09 | Mefentrifluconazole | 200:1 | 20:0.1 |
| X.09 | Mefentrifluconazole | 1:3 | 2:6 |
| X.09 | Mefentrifluconazole | 20:1 | 2:0.1 |
| X.09 | Florylpicoxamid | 20:1 | 20:1 |
| X.09 | Florylpicoxamid | 2:1 | 2:1 |
| X.11 | Pydiflumetofen | 1:3 | 2:6 |
| X.11 | Pydiflumetofen | 3.3:1 | 2:0.6 |
| X.11 | Pydiflumetofen | 1:30 | 0.2:6 |
| X.11 | Pydiflumetofen | 1:3 | 0.2:0.6 |
| X.11 | Benzovindiflupyr | 1:1 | 2:2 |
| X.1 | Benzovindiflupyr | 10:1 | 2:0.2 |
| X.11 | Benzovindiflupyr | 1:10 | 0.2:2 |
| X.11 | Benzovindiflupyr | 1:1 | 0.2:0.2 |
| X.11 | Cyprodinil | 1:10 | 2:20 |
| X.11 | Cyprodinil | 20:1 | 2:0.1 |
| X.11 | Cyprodinil | 1:100 | 0.2:20 |
| X.11 | Cyprodinil | 2:1 | 0.2:0.1 |
| X.11 | Fludioxonil | 1:3 | 2:6 |
| X.11 | Fludioxonil | 33.3:1 | 2:0.06 |
| X.11 | Fludioxonil | 1:30 | 0.2:6 |
| X.11 | Fludioxonil | 3.3:1 | 0.2:0.06 |
| X.11 | Azoxystrobin | 1:3 | 2:6 |
| X.11 | Azoxystrobin | 3.3:1 | 2:0.6 |
| X.11 | Azoxystrobin | 1:30 | 0.2:6 |
| X.11 | Azoxystrobin | 1:3 | 0.2:0.6 |
| X.11 | Difenoconazole | 1:3 | 2:6 |
| X.11 | Difenoconazole | 3.3:1 | 2:0.6 |
| X.11 | Difenoconazole | 1:30 | 0.2:6 |
| X.11 | Difenoconazole | 1:3 | 0.2:0.6 |
| X.11 | Prothioconazole | 3.3:1 | 2:0.6 |
| X.11 | Prothioconazole | 33.3:1 | 2:0.06 |
| X.11 | Prothioconazole | 1:3 | 0.2:0.6 |
| X.11 | Prothioconazole | 3.3:1 | 0.2:0.06 |
| X.11 | Mefentrifluconazole | 1:3 | 2:6 |
| X.11 | Mefentrifluconazole | 20:1 | 2:0.1 |
| X.11 | Mefentrifluconazole | 1:30 | 0.2:6 |
| X.11 | Mefentrifluconazole | 2:1 | 0.2:0.1 |
| X.11 | Florylpicoxamid | 2:1 | 2:1 |
| X.11 | Florylpicoxamid | 20:1 | 2:0.1 |
| X.11 | Florylpicoxamid | 1:5 | 0.2:1 |
| X.11 | Florylpicoxamid | 2:1 | 0.2:0.1 |
| X.11 | Acibenzolar-S-methyl | 1:10 | 2:20 |
| X.11 | Acibenzolar-S-methyl | 1:3 | 2:6 |
| X.11 | Acibenzolar-S-methyl | 1:100 | 0.2:20 |
| X.11 | Acibenzolar-S-methyl | 1:30 | 0.2:6 |
| X.12 | Pydiflumetofen | 1:3 | 2:6 |
| X.12 | Pydiflumetofen | 3.3:1 | 2:0.6 |
| X.12 | Pydiflumetofen | 1:30 | 0.2:6 |
| X.12 | Pydiflumetofen | 1:3 | 0.2:0.6 |
| X.12 | Benzovindiflupyr | 1:1 | 2:2 |
| X.12 | Benzovindiflupyr | 10:1 | 2:0.2 |
| X.12 | Benzovindiflupyr | 1:10 | 0.2:2 |
| X.12 | Benzovindiflupyr | 1:1 | 0.2:0.2 |
| X.12 | Cyprodinil | 1:10 | 2:20 |
| X.12 | Cyprodinil | 20:1 | 2:0.1 |
| X.12 | Cyprodinil | 1:100 | 0.2:20 |
| X.12 | Cyprodinil | 2:1 | 0.2:0.1 |
| X.12 | Fludioxonil | 1:3 | 2:6 |
| X.12 | Fludioxonil | 33.3:1 | 2:0.06 |
| X.12 | Fludioxonil | 1:30 | 0.2:6 |
| X.12 | Fludioxonil | 3.3:1 | 0.2:0.06 |
| X.12 | Azoxystrobin | 1:3 | 2:6 |
| X.12 | Azoxystrobin | 3.3:1 | 2:0.6 |
| X.12 | Azoxystrobin | 1:30 | 0.2:6 |
| X.12 | Azoxystrobin | 1:3 | 0.2:0.6 |
| X.12 | Difenoconazole | 1:3 | 2:6 |
| X.12 | Difenoconazole | 3.3:1 | 2:0.6 |
| X.12 | Difenoconazole | 1:30 | 0.2:6 |
| X.12 | Difenoconazole | 1:3 | 0.2:0.6 |
| X.12 | Prothioconazole | 3.3:1 | 2:0.6 |
| X.12 | Prothioconazole | 33.3:1 | 2:0.06 |
| X.12 | Prothioconazole | 1:3 | 0.2:0.6 |
| X.12 | Prothioconazole | 3.3:1 | 0.2:0.06 |
| X.12 | Mefentrifluconazole | 1:3 | 2:6 |
| X.12 | Mefentrifluconazole | 20:1 | 2:0.1 |
| X.12 | Mefentrifluconazole | 1:30 | 0.2:6 |
| X.12 | Mefentrifluconazole | 2:1 | 0.2:0.1 |
| X.12 | Florylpicoxamid | 2:1 | 2:1 |
| X.12 | Florylpicoxamid | 20:1 | 2:0.1 |
| X.12 | Florylpicoxamid | 1:5 | 0.2:1 |
| X.12 | Florylpicoxamid | 2:1 | 0.2:0.1 |
| X.12 | Acibenzolar-S-methyl | 1:10 | 2:20 |
| X.12 | Acibenzolar-S-methyl | 1:3 | 2:6 |
| X.12 | Acibenzolar-S-methyl | 1:100 | 0.2:20 |
| X.12 | Acibenzolar-S-methyl | 1:30 | 0.2:6 |
| X.13 | Pydiflumetofen | 1:30 | 0.2:6 |
| X.13 | Pydiflumetofen | 1:3 | 0.2:0.6 |
| X.13 | Pydiflumetofen | 1:300 | 0.02:6 |
| X.13 | Pydiflumetofen | 1:30 | 0.02:0.6 |
| X.13 | Benzovindiflupyr | 1:10 | 0.2:2 |
| X.13 | Benzovindiflupyr | 1:1 | 0.2:0.2 |
| X.13 | Benzovindiflupyr | 1:100 | 0.02:2 |
| X.13 | Benzovindiflupyr | 1:10 | 0.02:0.2 |
| X.13 | Cyprodinil | 1:100 | 0.2:20 |
| X.13 | Cyprodinil | 2:1 | 0.2:0.1 |
| X.13 | Cyprodinil | 1:1000 | 0.02:20 |
| X.13 | Cyprodinil | 1:5 | 0.02:0.1 |
| X.13 | Fludioxonil | 1:30 | 0.2:6 |
| X.13 | Fludioxonil | 3.3:1 | 0.2:0.06 |
| X.13 | Fludioxonil | 1:300 | 0.02:6 |
| X.13 | Fludioxonil | 1:3 | 0.02:0.06 |
| X.13 | Azoxystrobin | 1:30 | 0.2:6 |
| X.13 | Azoxystrobin | 1:3 | 0.2:0.6 |
| X.13 | Azoxystrobin | 1:300 | 0.02:6 |
| X.13 | Azoxystrobin | 1:30 | 0.02:0.6 |
| X.13 | Difenoconazole | 1:30 | 0.2:6 |
| X.13 | Difenoconazole | 1:3 | 0.2:0.6 |
| X.13 | Difenoconazole | 1:300 | 0.02:6 |
| X.13 | Difenoconazole | 1:30 | 0.02:0.6 |
| X.13 | Prothioconazole | 1:3 | 0.2:0.6 |
| X.13 | Prothioconazole | 3.3:1 | 0.2:0.06 |
| X.13 | Prothioconazole | 1:30 | 0.02:0.6 |
| X.13 | Prothioconazole | 1:3 | 0.02:0.06 |
| X.13 | Mefentrifluconazole | 1:30 | 0.2:6 |
| X.13 | Mefentrifluconazole | 2:1 | 0.2:0.1 |
| X.13 | Mefentrifluconazole | 1:300 | 0.02:6 |
| X.13 | Mefentrifluconazole | 1:5 | 0.02:0.1 |
| X.13 | Florylpicoxamid | 1:5 | 0.2:1 |
| X.13 | Florylpicoxamid | 2:1 | 0.2:0.1 |
| X.13 | Florylpicoxamid | 1:50 | 0.02:1 |
| X.13 | Florylpicoxamid | 1:5 | 0.02:0.1 |
| X.13 | Acibenzolar-S-methyl | 1:100 | 0.2:20 |
| X.13 | Acibenzolar-S-methyl | 1:30 | 0.2:6 |
| X.15 | Pydiflumetofen | 1:3 | 2:6 |
| X.15 | Pydiflumetofen | 3.3:1 | 2:0.6 |
| X.15 | Pydiflumetofen | 1:10 | 0.6:6 |
| X.15 | Pydiflumetofen | 1:1 | 0.6:0.6 |
| X.15 | Benzovindiflupyr | 1:1 | 2:2 |
| X.15 | Benzovindiflupyr | 10:1 | 2:0.2 |
| X.15 | Benzovindiflupyr | 1:3.3 | 0.6:2 |
| X.15 | Benzovindiflupyr | 3:1 | 0.6:0.2 |
| X.15 | Cyprodinil | 1:10 | 2:20 |
| X.15 | Cyprodinil | 20:1 | 2:0.1 |
| X.15 | Cyprodinil | 1:33.3 | 0.6:20 |
| X.15 | Cyprodinil | 6:1 | 0.6:0.1 |
| X.15 | Fludioxonil | 1:3 | 2:6 |
| X.15 | Fludioxonil | 33.3:1 | 2:0.06 |
| X.15 | Fludioxonil | 1:10 | 0.6:6 |
| X.15 | Fludioxonil | 10:1 | 0.6:0.06 |
| X.15 | Azoxystrobin | 1:3 | 2:6 |
| X.15 | Azoxystrobin | 3.3:1 | 2:0.6 |
| X.15 | Azoxystrobin | 1:10 | 0.6:6 |
| X.15 | Azoxystrobin | 1:1 | 0.6:0.6 |
| X.15 | Difenoconazole | 1:3 | 2:6 |
| X.15 | Difenoconazole | 3.3:1 | 2:0.6 |
| X.15 | Difenoconazole | 1:10 | 0.6:6 |
| X.15 | Difenoconazole | 1:1 | 0.6:0.6 |
| X.15 | Prothioconazole | 3.3:1 | 2:0.6 |
| X.15 | Prothioconazole | 33.3:1 | 2:0.06 |
| X.15 | Prothioconazole | 1:1 | 0.6:0.6 |
| X.15 | Prothioconazole | 10:1 | 0.6:0.06 |
| X.15 | Mefentrifluconazole | 1:3 | 2:6 |
| X.15 | Mefentrifluconazole | 20:1 | 2:0.1 |
| X.15 | Mefentrifluconazole | 1:10 | 0.6:6 |
| X.15 | Mefentrifluconazole | 6:1 | 0.6:0.1 |
| X.15 | Florylpicoxamid | 2:1 | 2:1 |
| X.15 | Florylpicoxamid | 20:1 | 2:0.1 |
| X.15 | Florylpicoxamid | 1:1.6 | 0.6:1 |
| X.15 | Florylpicoxamid | 6:1 | 0.6:0.1 |
| X.15 | Acibenzolar-S-methyl | 1:10 | 2:20 |
| X.15 | Acibenzolar-S-methyl | 1:3 | 2:6 |
| X.15 | Acibenzolar-S-methyl | 1:33.3 | 0.6:20 |
| X.15 | Acibenzolar-S-methyl | 1:10 | 0.6:6 |
| X.16 | Pydiflumetofen | 1:1 | 6:6 |
| X.16 | Pydiflumetofen | 10:1 | 6:0.6 |
| X.16 | Pydiflumetofen | 1:6 | 1:6 |
| X.16 | Pydiflumetofen | 1.6:1 | 1:0.6 |
| X.16 | Benzovindiflupyr | 3:1 | 6:2 |
| X.16 | Benzovindiflupyr | 30:1 | 6:0.2 |
| X.16 | Benzovindiflupyr | 1:2 | 1:2 |
| X.16 | Benzovindiflupyr | 5:1 | 1:0.2 |
| X.16 | Cyprodinil | 1:3.3 | 6:20 |
| X.16 | Cyprodinil | 60:1 | 6:0.1 |
| X.16 | Cyprodinil | 1:20 | 1:20 |
| X.16 | Cyprodinil | 10:1 | 1:0.1 |
| X.16 | Fludioxonil | 1:1 | 6:6 |
| X.16 | Fludioxonil | 100:1 | 6:0.06 |
| X.16 | Fludioxonil | 1:6 | 1:6 |
| X.16 | Fludioxonil | 16.6:1 | 1:0.6 |
| X.16 | Azoxystrobin | 1:1 | 6:6 |
| X.16 | Azoxystrobin | 10:1 | 6:0.6 |
| X.16 | Azoxystrobin | 1:6 | 1:6 |
| X.16 | Azoxystrobin | 1.6:1 | 1:0.6 |
| X.16 | Difenoconazole | 1:1 | 6:6 |
| X.16 | Difenoconazole | 10:1 | 6:0.6 |
| X.16 | Difenoconazole | 1:6 | 1:6 |
| X.16 | Difenoconazole | 1.6:1 | 1:0.6 |
| X.16 | Prothioconazole | 10:1 | 6:0.6 |
| X.16 | Prothioconazole | 100:1 | 6:0.06 |
| X.16 | Prothioconazole | 1.6:1 | 1:0.6 |
| X.16 | Mefentrifluconazole | 1:1 | 6:6 |
| X.16 | Mefentrifluconazole | 60:1 | 6:0.1 |
| X.16 | Mefentrifluconazole | 1:6 | 1:6 |
| X.16 | Mefentrifluconazole | 10:1 | 1:0.1 |
| X.16 | Florylpicoxamid | 6:1 | 6:1 |
| X.16 | Florylpicoxamid | 60:1 | 6:0.1 |
| X.16 | Florylpicoxamid | 1:1 | 1:1 |
| X.16 | Acibenzolar-S-methyl | 1:3.3 | 6:20 |
| X.16 | Acibenzolar-S-methyl | 1:1 | 6:6 |
| X.14 | Pydiflumetofen | 3.3:1 | 20:6 |
| X.14 | Pydiflumetofen | 33.3:1 | 20:0.6 |
| X.14 | Pydiflumetofen | 1:1 | 6:6 |
| X.14 | Pydiflumetofen | 10:1 | 6:0.6 |
| X.14 | Benzovindiflupyr | 10:1 | 20:2 |
| X.14 | Benzovindiflupyr | 100:1 | 20:0.2 |
| X.14 | Benzovindiflupyr | 3:1 | 6:2 |
| X.14 | Benzovindiflupyr | 30:1 | 6:0.2 |
| X.14 | Cyprodinil | 1:1 | 20:20 |
| X.14 | Cyprodinil | 200:1 | 20:0.1 |
| X.14 | Cyprodinil | 1:3.3 | 6:20 |
| X.14 | Cyprodinil | 60:1 | 6:0.1 |
| X.14 | Fludioxonil | 3.3:1 | 20:6 |
| X.14 | Fludioxonil | 333.3:1 | 20:0.06 |
| X.14 | Fludioxonil | 1:1 | 6:6 |
| X.14 | Fludioxonil | 100:1 | 6:0.06 |
| X.14 | Azoxystrobin | 3.3:1 | 20:6 |
| X.14 | Azoxystrobin | 33.3:1 | 20:0.6 |
| X.14 | Azoxystrobin | 1:1 | 6:6 |
| X.14 | Azoxystrobin | 10:1 | 6:0.6 |
| X.14 | Difenoconazole | 3.3:1 | 20:6 |
| X.14 | Difenoconazole | 33.3:1 | 20:0.6 |
| X.14 | Difenoconazole | 1:1 | 6:6 |
| X.14 | Difenoconazole | 10:1 | 6:0.6 |
| X.14 | Prothioconazole | 33.3:1 | 20:0.6 |
| X.14 | Prothioconazole | 333.3:1 | 20:0.06 |
| X.14 | Prothioconazole | 10:1 | 6:0.6 |
| X.14 | Prothioconazole | 100:1 | 6:0.06 |
| X.14 | Mefentrifluconazole | 3.3:1 | 20:6 |
| X.14 | Mefentrifluconazole | 200:1 | 20:0.1 |
| X.14 | Mefentrifluconazole | 1:1 | 6:6 |
| X.14 | Mefentrifluconazole | 60:1 | 6:0.1 |
| X.14 | Florylpicoxamid | 20:1 | 20:1 |
| X.14 | Florylpicoxamid | 200:1 | 20:0.1 |
| X.14 | Florylpicoxamid | 6:1 | 6:1 |
| X.14 | Florylpicoxamid | 60:1 | 6:0.1 |
| X.14 | Acibenzolar-S-methyl | 1:1 | 20:20 |
| X.14 | Acibenzolar-S-methyl | 3.3:1 | 20:6 |
| X.14 | Acibenzolar-S-methyl | 1:1 | 6:6 |

### Example M-B2: Glomerella lagenarium syn. Colletotrichum lagenarium (anthracnose of cucurbits)

Conidia of the fungus, prepared from a freshly cultivated petri dish, were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically and visually after 72 hrs.

The following mixture compositions (A:B) at the X.05 Benzovindiflupyr 1:10 0.2:2 reported concentration (in ppm) gave at least 70% X.05 Benzovindiflupyr 1:1 0.2:0.2 disease control in the test system.

| | | | Conc. |
|---|---|---|---|
| Comp. | | Ratio | (ppm) |
| A | Component B | A:B | (A:B) |
| X.01 | Pydiflumetofen | 1:30 | 0.2:6 |
| X.01 | Pydiflumetofen | 1:3 | 0.2:0.6 |
| X.01 | Benzovindiflupyr | 1:10 | 0.2:2 |
| X.01 | Benzovindiflupyr | 1:1 | 0.2:0.2 |
| X.01 | Benzovindiflupyr | 1:33.3 | 0.06:2 |
| X.01 | Benzovindiflupyr | 1:3.3 | 0.06:0.2 |
| X.01 | Cyprodinil | 1:100 | 0.2:20 |
| X.01 | Cyprodinil | 2:1 | 0.2:0.1 |
| X.01 | Cyprodinil | 1:333.3 | 0.06:20 |
| X.01 | Fludioxonil | 1:30 | 0.2:6 |
| X.01 | Fludioxonil | 3.3:1 | 0.2:0.06 |
| X.01 | Fludioxonil | 1:100 | 0.06:6 |
| X.01 | Fludioxonil | 1:1 | 0.06:0.06 |
| X.01 | Azoxystrobin | 1:30 | 0.2:6 |
| X.01 | Azoxystrobin | 1:3 | 0.2:0.6 |
| X.01 | Azoxystrobin | 1:100 | 0.06:6 |
| X.01 | Azoxystrobin | 1:10 | 0.06:0.6 |
| X.01 | Difenoconazole | 1:30 | 0.2:6 |
| X.01 | Difenoconazole | 1:3 | 0.2:0.6 |
| X.01 | Difenoconazole | 1:100 | 0.06:6 |
| X.01 | Difenoconazole | 1:10 | 0.06:0.6 |
| X.01 | Prothioconazole | 1:3 | 0.2:0.6 |
| X.01 | Prothioconazole | 3.3:1 | 0.2:0.06 |
| X.01 | Prothioconazole | 1:10 | 0.06:0.6 |
| X.01 | Prothioconazole | 1:1 | 0.06:0.06 |
| X.01 | Mefentrifluconazole | 1:30 | 0.2:6 |
| X.01 | Mefentrifluconazole | 2:1 | 0.2:0.1 |
| X.01 | Mefentrifluconazole | 1:100 | 0.06:6 |
| X.01 | Florylpicoxamid | 1:5 | 0.2:1 |
| X.01 | Florylpicoxamid | 2:1 | 0.2:0.1 |
| X.01 | Acibenzolar-S-methyl | 1:100 | 0.2:20 |
| X.01 | Acibenzolar-S-methyl | 1:30 | 0.2:6 |
| X.01 | Acibenzolar-S-methyl | 1:333.3 | 0.06:20 |
| X.05 | Pydiflumetofen | 1:30 | 0.2:6 |
| X.05 | Pydiflumetofen | 1:3 | 0.2:0.6 |
| X.05 | Benzovindiflupyr | 1:10 | 0.2:2 |
| X.05 | Benzovindiflupyr | 1:1 | 0.2:0.2 |
| X.05 | Benzovindiflupyr | 1:33.3 | 0.06:2 |
| X.05 | Benzovindiflupyr | 1:3.3 | 0.06:0.2 |
| X.05 | Cyprodinil | 1:100 | 0.2:20 |
| X.05 | Cyprodinil | 2:1 | 0.2:0.1 |
| X.05 | Cyprodinil | 1:333.3 | 0.06:20 |
| X.05 | Fludioxonil | 1:30 | 0.2:6 |
| X.05 | Fludioxonil | 3.3:1 | 0.2:0.06 |
| X.05 | Fludioxonil | 1:100 | 0.06:6 |
| X.05 | Fludioxonil | 1:1 | 0.06:0.06 |
| X.05 | Azoxystrobin | 1:30 | 0.2:6 |
| X.05 | Azoxystrobin | 1:3 | 0.2:0.6 |
| X.05 | Azoxystrobin | 1:100 | 0.06:6 |
| X.05 | Azoxystrobin | 1:10 | 0.06:0.6 |
| X.05 | Difenoconazole | 1:30 | 0.2:6 |
| X.05 | Difenoconazole | 1:3 | 0.2:0.6 |
| X.05 | Difenoconazole | 1:100 | 0.06:6 |
| X.05 | Difenoconazole | 1:10 | 0.06:0.6 |
| X.05 | Prothioconazole | 1:3 | 0.2:0.6 |
| X.05 | Prothioconazole | 3.3:1 | 0.2:0.06 |
| X.05 | Prothioconazole | 1:10 | 0.06:0.6 |
| X.05 | Prothioconazole | 1:1 | 0.06:0.06 |
| X.05 | Mefentrifluconazole | 1:30 | 0.2:6 |
| X.05 | Mefentrifluconazole | 2:1 | 0.2:0.1 |
| X.05 | Mefentrifluconazole | 1:100 | 0.06:6 |
| X.05 | Mefentrifluconazole | 1:1.6 | 0.06:0.1 |
| X.05 | Florylpicoxamid | 1:5 | 0.2:1 |
| X.05 | Florylpicoxamid | 2:1 | 0.2:0.1 |
| X.05 | Florylpicoxamid | 1:16.6 | 0.06:1 |
| X.05 | Florylpicoxamid | 1:1.6 | 0.06:0.1 |
| X.05 | Acibenzolar-S-methyl | 1:100 | 0.2:20 |
| X.05 | Acibenzolar-S-methyl | 1:30 | 0.2:6 |
| X.05 | Acibenzolar-S-methyl | 1:333.3 | 0.06:20 |
| X.05 | Acibenzolar-S-methyl | 1:100 | 0.06:6 |
| X.03 | Pydiflumetofen | 1:1 | 6:6 |
| X.03 | Pydiflumetofen | 10:1 | 6:0.6 |
| X.03 | Pydiflumetofen | 1:6 | 1:6 |
| X.03 | Pydiflumetofen | 1.6:1 | 1:0.6 |
| X.03 | Benzovindiflupyr | 3:1 | 6:2 |
| X.03 | Benzovindiflupyr | 30:1 | 6:0.2 |
| X.03 | Benzovindiflupyr | 1:2 | 1:2 |
| X.03 | Benzovindiflupyr | 5:1 | 1:0.2 |
| X.03 | Cyprodinil | 1:3.3 | 6:20 |
| X.03 | Cyprodinil | 60:1 | 6:0.1 |
| X.03 | Cyprodinil | 1:20 | 1:20 |
| X.03 | Cyprodinil | 10:1 | 1:0.1 |
| X.03 | Fludioxonil | 1:1 | 6:6 |
| X.03 | Fludioxonil | 100:1 | 6:0.06 |
| X.03 | Fludioxonil | 1:6 | 1:6 |
| X.03 | Fludioxonil | 16.6:1 | 1:0.6 |
| X.03 | Azoxystrobin | 1:1 | 6:6 |
| X.03 | Azoxystrobin | 10:1 | 6:0.6 |
| X.03 | Azoxystrobin | 1:6 | 1:6 |
| X.03 | Azoxystrobin | 1.6:1 | 1:0.6 |
| X.03 | Difenoconazole | 1:1 | 6:6 |
| X.03 | Difenoconazole | 10:1 | 6:0.6 |
| X.03 | Difenoconazole | 1:6 | 1:6 |
| X.03 | Difenoconazole | 1.6:1 | 1:0.6 |
| X.03 | Prothioconazole | 10:1 | 6:0.6 |
| X.03 | Prothioconazole | 100:1 | 6:0.06 |
| X.03 | Prothioconazole | 1.6:1 | 1:0.6 |
| X.03 | Prothioconazole | 16.6:1 | 1:0.6 |
| X.03 | Mefentrifluconazole | 1:1 | 6:6 |
| X.03 | Mefentrifluconazole | 60:1 | 6:0.1 |
| X.03 | Mefentrifluconazole | 1:6 | 1:6 |
| X.03 | Mefentrifluconazole | 10:1 | 1:0.1 |
| X.03 | Florylpicoxamid | 6:1 | 6:1 |
| X.03 | Florylpicoxamid | 60:1 | 6:0.1 |
| X.03 | Florylpicoxamid | 1:1 | 1:1 |
| X.03 | Florylpicoxamid | 10:1 | 1:0.1 |
| X.03 | Acibenzolar-S-methyl | 1:3.3 | 6:20 |
| X.03 | Acibenzolar-S-methyl | 1:1 | 6:6 |
| X.03 | Acibenzolar-S-methyl | 1:6 | 1:6 |
| X.08 | Pydiflumetofen | 1:1 | 6:6 |
| X.08 | Pydiflumetofen | 10:1 | 6:0.6 |
| X.08 | Pydiflumetofen | 1:6 | 1:6 |
| X.08 | Pydiflumetofen | 1.6:1 | 1:0.6 |
| X.08 | Benzovindiflupyr | 3:1 | 6:2 |
| X.08 | Benzovindiflupyr | 30:1 | 6:0.2 |
| X.08 | Benzovindiflupyr | 1:2 | 1:2 |
| X.08 | Benzovindiflupyr | 5:1 | 1:0.2 |
| X.08 | Cyprodinil | 1:3.3 | 6:20 |
| X.08 | Cyprodinil | 60:1 | 6:0.1 |
| X.08 | Cyprodinil | 1:20 | 1:20 |
| X.08 | Cyprodinil | 10:1 | 1:0.1 |
| X.08 | Fludioxonil | 1:1 | 6:6 |
| X.08 | Fludioxonil | 100:1 | 6:0.06 |
| X.08 | Fludioxonil | 1:6 | 1:6 |
| X.08 | Fludioxonil | 16.6:1 | 1:0.6 |
| X.08 | Azoxystrobin | 1:1 | 6:6 |
| X.08 | Azoxystrobin | 10:1 | 6:0.6 |
| X.08 | Azoxystrobin | 1:6 | 1:6 |
| X.08 | Azoxystrobin | 1.6:1 | 1:0.6 |
| X.08 | Difenoconazole | 1:1 | 6:6 |
| X.08 | Difenoconazole | 10:1 | 6:0.6 |
| X.08 | Difenoconazole | 1:6 | 1:6 |
| X.08 | Difenoconazole | 1.6:1 | 1:0.6 |
| X.08 | Prothioconazole | 10:1 | 6:0.6 |
| X.08 | Prothioconazole | 100:1 | 6:0.06 |
| X.08 | Prothioconazole | 1.6:1 | 1:0.6 |
| X.08 | Prothioconazole | 16.6:1 | 1:0.6 |
| X.08 | Mefentrifluconazole | 1:1 | 6:6 |
| X.08 | Mefentrifluconazole | 60:1 | 6:0.1 |
| X.08 | Mefentrifluconazole | 1:6 | 1:6 |
| X.08 | Mefentrifluconazole | 10:1 | 1:0.1 |
| X.08 | Florylpicoxamid | 6:1 | 6:1 |
| X.08 | Florylpicoxamid | 60:1 | 6:0.1 |
| X.08 | Florylpicoxamid | 1:1 | 1:1 |
| X.08 | Florylpicoxamid | 10:1 | 1:0.1 |
| X.08 | Acibenzolar-S-methyl | 1:3.3 | 6:20 |
| X.08 | Acibenzolar-S-methyl | 1:1 | 6:6 |
| X.08 | Acibenzolar-S-methyl | 1:20 | 1:20 |
| X.08 | Acibenzolar-S-methyl | 1:6 | 1:6 |
| X.10 | Pydiflumetofen | 1:1 | 6:6 |
| X.10 | Pydiflumetofen | 10:1 | 6:0.6 |
| X.10 | Benzovindiflupyr | 3:1 | 6:2 |
| X.10 | Benzovindiflupyr | 30:1 | 6:0.2 |
| X.10 | Benzovindiflupyr | 1:2 | 1:2 |
| X.10 | Benzovindiflupyr | 5:1 | 1:0.2 |
| X.10 | Cyprodinil | 1:3.3 | 6:20 |
| X.10 | Cyprodinil | 60:1 | 6:0.1 |
| X.10 | Cyprodinil | 1:20 | 1:20 |
| X.10 | Fludioxonil | 1:1 | 6:6 |
| X.10 | Fludioxonil | 100:1 | 6:0.06 |
| X.10 | Fludioxonil | 1:6 | 1:6 |
| X.10 | Azoxystrobin | 1:1 | 6:6 |
| X.10 | Azoxystrobin | 10:1 | 6:0.6 |
| X.10 | Azoxystrobin | 1:6 | 1:6 |
| X.10 | Azoxystrobin | 1.6:1 | 1:0.6 |
| X.10 | Difenoconazole | 1:1 | 6:6 |
| X.10 | Difenoconazole | 10:1 | 6:0.6 |
| X.10 | Difenoconazole | 1:6 | 1:6 |
| X.10 | Prothioconazole | 10:1 | 6:0.6 |
| X.10 | Prothioconazole | 100:1 | 6:0.06 |
| X.10 | Prothioconazole | 1.6:1 | 1:0.6 |
| X.10 | Prothioconazole | 16.6:1 | 1:0.6 |
| X.10 | Mefentrifluconazole | 1:1 | 6:6 |
| X.10 | Mefentrifluconazole | 60:1 | 6:0.1 |
| X.10 | Florylpicoxamid | 6:1 | 6:1 |
| X.10 | Florylpicoxamid | 60:1 | 6:0.1 |
| X.10 | Acibenzolar-S-methyl | 1:3.3 | 6:20 |
| X.10 | Acibenzolar-S-methyl | 1:1 | 6:6 |
| X.10 | Acibenzolar-S-methyl | 1:20 | 1:20 |
| X.02 | Pydiflumetofen | 1:3 | 2:6 |
| X.02 | Pydiflumetofen | 3.3:1 | 2:0.6 |
| X.02 | Pydiflumetofen | 1:10 | 0.6:6 |
| X.02 | Pydiflumetofen | 1:1 | 0.6:0.6 |
| X.02 | Benzovindiflupyr | 1:1 | 2:2 |
| X.02 | Benzovindiflupyr | 10:1 | 2:0.2 |
| X.02 | Benzovindiflupyr | 1:3.3 | 0.6:2 |
| X.02 | Benzovindiflupyr | 3:1 | 0.6:0.2 |
| X.02 | Cyprodinil | 1:10 | 2:20 |
| X.02 | Cyprodinil | 20:1 | 2:0.1 |
| X.02 | Cyprodinil | 1:33.3 | 0.6:20 |
| X.02 | Cyprodinil | 6:1 | 0.6:0.1 |
| X.02 | Fludioxonil | 1:3 | 2:6 |
| X.02 | Fludioxonil | 33.3:1 | 2:0.06 |
| X.02 | Fludioxonil | 1:10 | 0.6:6 |
| X.02 | Fludioxonil | 10:1 | 0.6:0.06 |
| X.02 | Azoxystrobin | 1:3 | 2:6 |
| X.02 | Azoxystrobin | 3.3:1 | 2:0.6 |
| X.02 | Azoxystrobin | 1:10 | 0.6:6 |
| X.02 | Azoxystrobin | 1:1 | 0.6:0.6 |
| X.02 | Difenoconazole | 1:3 | 2:6 |
| X.02 | Difenoconazole | 3.3:1 | 2:0.6 |
| X.02 | Difenoconazole | 1:10 | 0.6:6 |
| X.02 | Difenoconazole | 1:1 | 0.6:0.6 |
| X.02 | Prothioconazole | 3.3:1 | 2:0.6 |
| X.02 | Prothioconazole | 33.3:1 | 2:0.06 |
| X.02 | Prothioconazole | 1:1 | 0.6:0.6 |
| X.02 | Prothioconazole | 10:1 | 0.6:0.06 |
| X.02 | Mefentrifluconazole | 1:3 | 2:6 |
| X.02 | Mefentrifluconazole | 20:1 | 2:0.1 |
| X.02 | Mefentrifluconazole | 1:10 | 0.6:6 |
| X.02 | Mefentrifluconazole | 6:1 | 0.6:0.1 |
| X.02 | Florylpicoxamid | 2:1 | 2:1 |
| X.02 | Florylpicoxamid | 20:1 | 2:0.1 |
| X.02 | Florylpicoxamid | 1:1.6 | 0.6:1 |
| X.02 | Florylpicoxamid | 6:1 | 0.6:0.1 |
| X.02 | Acibenzolar-S-methyl | 1:10 | 2:20 |
| X.02 | Acibenzolar-S-methyl | 1:3 | 2:6 |
| X.02 | Acibenzolar-S-methyl | 1:33.3 | 0.6:20 |
| X.02 | Acibenzolar-S-methyl | 1:10 | 0.6:6 |
| X.04 | Benzovindiflupyr | 3:1 | 6:2 |
| X.04 | Benzovindiflupyr | 30:1 | 6:0.2 |
| X.04 | Benzovindiflupyr | 1:3.3 | 0.6:2 |
| X.04 | Benzovindiflupyr | 3:1 | 0.6:0.2 |
| X.04 | Fludioxonil | 1:1 | 6:6 |
| X.04 | Fludioxonil | 100:1 | 6:0.06 |
| X.04 | Azoxystrobin | 1:1 | 6:6 |
| X.04 | Azoxystrobin | 10:1 | 6:0.6 |
| X.04 | Azoxystrobin | 1:10 | 0.6:6 |
| X.04 | Azoxystrobin | 1:1 | 0.6:0.6 |
| X.04 | Difenoconazole | 1:1 | 6:6 |
| X.04 | Difenoconazole | 10:1 | 6:0.6 |
| X.04 | Prothioconazole | 10:1 | 6:0.6 |
| X.04 | Prothioconazole | 1:1 | 0.6:0.6 |
| X.04 | Prothioconazole | 10:1 | 0.6:0.06 |
| X.06 | Pydiflumetofen | 3.3:1 | 20:6 |
| X.06 | Pydiflumetofen | 33.3:1 | 20:0.6 |
| X.06 | Benzovindiflupyr | 10:1 | 20:2 |
| X.06 | Benzovindiflupyr | 100:1 | 20:0.2 |
| X.06 | Benzovindiflupyr | 1:2 | 1:2 |
| X.06 | Benzovindiflupyr | 5:1 | 1:0.2 |
| X.06 | Cyprodinil | 1:1 | 20:20 |
| X.06 | Cyprodinil | 200:1 | 20:0.1 |
| X.06 | Cyprodinil | 1:20 | 1:20 |
| X.06 | Fludioxonil | 3.3:1 | 20:6 |
| X.06 | Fludioxonil | 333.3:1 | 20:0.06 |
| X.06 | Fludioxonil | 1:6 | 1:6 |
| X.06 | Fludioxonil | 16.6:1 | 1:0.6 |
| X.06 | Azoxystrobin | 3.3:1 | 20:6 |
| X.06 | Azoxystrobin | 33.3:1 | 20:0.6 |
| X.06 | Azoxystrobin | 1:6 | 1:6 |
| X.06 | Azoxystrobin | 1.6:1 | 1:0.6 |
| X.06 | Difenoconazole | 3.3:1 | 20:6 |
| X.06 | Difenoconazole | 33.3:1 | 20:0.6 |
| X.06 | Difenoconazole | 1:6 | 1:6 |
| X.06 | Difenoconazole | 1.6:1 | 1:0.6 |
| X.06 | Prothioconazole | 33.3:1 | 20:0.6 |
| X.06 | Prothioconazole | 333.3:1 | 20:0.06 |
| X.06 | Prothioconazole | 1.6:1 | 1:0.6 |
| X.06 | Prothioconazole | 16.6:1 | 1:0.6 |
| X.06 | Mefentrifluconazole | 3.3:1 | 20:6 |
| X.06 | Mefentrifluconazole | 200:1 | 20:0.1 |
| X.06 | Mefentrifluconazole | 1:6 | 1:6 |
| X.06 | Florylpicoxamid | 20:1 | 20:1 |
| X.06 | Florylpicoxamid | 200:1 | 20:0.1 |
| X.06 | Florylpicoxamid | 1:1 | 1:1 |
| X.06 | Florylpicoxamid | 10:1 | 1:0.1 |
| X.06 | Acibenzolar-S-methyl | 1:1 | 20:20 |
| X.06 | Acibenzolar-S-methyl | 3.3:1 | 20:6 |
| X.06 | Acibenzolar-S-methyl | 1:20 | 1:20 |
| X.06 | Acibenzolar-S-methyl | 1:6 | 1:6 |
| X.07 | Pydiflumetofen | 1:6 | 1:6 |
| X.07 | Pydiflumetofen | 1.6:1 | 1:0.6 |
| X.07 | Benzovindiflupyr | 1:2 | 1:2 |
| X.07 | Benzovindiflupyr | 5:1 | 1:0.2 |
| X.07 | Benzovindiflupyr | 1:20 | 0.1:2 |
| X.07 | Benzovindiflupyr | 1:2 | 0.1:0.2 |
| X.07 | Cyprodinil | 1:20 | 1:20 |
| X.07 | Cyprodinil | 10:1 | 1:0.1 |
| X.07 | Cyprodinil | 1:200 | 0.1:20 |
| X.07 | Fludioxonil | 1:6 | 1:6 |
| X.07 | Fludioxonil | 16.6:1 | 1:0.6 |
| X.07 | Fludioxonil | 1:60 | 0.1:6 |
| X.07 | Fludioxonil | 1.6:1 | 0.1:0.06 |
| X.07 | Azoxystrobin | 1:6 | 1:6 |
| X.07 | Azoxystrobin | 1.6:1 | 1:0.6 |
| X.07 | Azoxystrobin | 1:6 | 0.1:6 |
| X.07 | Azoxystrobin | 1:6 | 0.1:0.6 |
| X.07 | Difenoconazole | 1:6 | 1:6 |
| X.07 | Difenoconazole | 1.6:1 | 1:0.6 |
| X.07 | Difenoconazole | 1:60 | 0.1:6 |
| X.07 | Difenoconazole | 1:6 | 0.1:0.6 |
| X.07 | Prothioconazole | 1.6:1 | 1:0.6 |
| X.07 | Prothioconazole | 16.6:1 | 1:0.6 |
| X.07 | Prothioconazole | 1:6 | 0.1:0.6 |
| X.07 | Prothioconazole | 1.6:1 | 0.1:0.06 |
| X.07 | Mefentrifluconazole | 1:6 | 1:6 |
| X.07 | Mefentrifluconazole | 10:1 | 1:0.1 |
| X.07 | Florylpicoxamid | 1:1 | 1:1 |
| X.07 | Florylpicoxamid | 10:1 | 1:0.1 |
| X.07 | Acibenzolar-S-methyl | 1:20 | 1:20 |
| X.07 | Acibenzolar-S-methyl | 1:6 | 1:6 |
| X.07 | Acibenzolar-S-methyl | 1:200 | 0.1:20 |
| X.09 | Benzovindiflupyr | 10:1 | 20:2 |
| X.09 | Benzovindiflupyr | 100:1 | 20:0.2 |
| X.09 | Benzovindiflupyr | 1:1 | 2:2 |
| X.09 | Benzovindiflupyr | 10:1 | 2:0.2 |
| X.09 | Azoxystrobin | 3.3:1 | 20:6 |
| X.09 | Azoxystrobin | 33.3:1 | 20:0.6 |
| X.09 | Azoxystrobin | 1:3 | 2:6 |
| X.09 | Azoxystrobin | 3.3:1 | 2:0.6 |
| X.09 | Difenoconazole | 3.3:1 | 20:6 |
| X.09 | Difenoconazole | 1:3 | 2:6 |
| X.09 | Prothioconazole | 33.3:1 | 20:0.6 |
| X.09 | Prothioconazole | 333.3:1 | 20:0.06 |
| X.09 | Prothioconazole | 3.3:1 | 2:0.6 |
| X.09 | Prothioconazole | 33.3:1 | 2:0.06 |
| X.11 | Pydiflumetofen | 1:3 | 2:6 |
| X.11 | Pydiflumetofen | 3.3:1 | 2:0.6 |
| X.11 | Pydiflumetofen | 1:30 | 0.2:6 |
| X.11 | Pydiflumetofen | 1:3 | 0.2:0.6 |
| X.11 | Benzovindiflupyr | 1:1 | 2:2 |
| X.11 | Benzovindiflupyr | 10:1 | 2:0.2 |
| X.11 | Benzovindiflupyr | 1:10 | 0.2:2 |
| X.11 | Benzovindiflupyr | 1:1 | 0.2:0.2 |
| X.11 | Cyprodinil | 1:10 | 2:20 |
| X.11 | Cyprodinil | 20:1 | 2:0.1 |
| X.11 | Cyprodinil | 1:100 | 0.2:20 |
| X.11 | Cyprodinil | 2:1 | 0.2:0.1 |
| X.11 | Fludioxonil | 1:3 | 2:6 |
| X.11 | Fludioxonil | 33.3:1 | 2:0.06 |
| X.11 | Fludioxonil | 1:30 | 0.2:6 |
| X.11 | Fludioxonil | 3.3:1 | 0.2:0.06 |
| X.11 | Azoxystrobin | 1:3 | 2:6 |
| X.11 | Azoxystrobin | 3.3:1 | 2:0.6 |
| X.11 | Azoxystrobin | 1:30 | 0.2:6 |
| X.11 | Azoxystrobin | 1:3 | 0.2:0.6 |
| X.11 | Difenoconazole | 1:3 | 2:6 |
| X.11 | Difenoconazole | 3.3:1 | 2:0.6 |
| X.11 | Difenoconazole | 1:30 | 0.2:6 |
| X.11 | Difenoconazole | 1:3 | 0.2:0.6 |
| X.11 | Prothioconazole | 3.3:1 | 2:0.6 |
| X.11 | Prothioconazole | 33.3:1 | 2:0.06 |
| X.11 | Prothioconazole | 1:3 | 0.2:0.6 |
| X.11 | Prothioconazole | 3.3:1 | 0.2:0.06 |
| X.11 | Mefentrifluconazole | 1:3 | 2:6 |
| X.11 | Mefentrifluconazole | 20:1 | 2:0.1 |
| X.11 | Mefentrifluconazole | 1:30 | 0.2:6 |
| X.11 | Mefentrifluconazole | 2:1 | 0.2:0.1 |
| X.11 | Florylpicoxamid | 2:1 | 2:1 |
| X.11 | Florylpicoxamid | 20:1 | 2:0.1 |
| X.11 | Florylpicoxamid | 1:5 | 0.2:1 |
| X.11 | Florylpicoxamid | 2:1 | 0.2:0.1 |
| X.11 | Acibenzolar-S-methyl | 1:10 | 2:20 |
| X.11 | Acibenzolar-S-methyl | 1:3 | 2:6 |
| X.11 | Acibenzolar-S-methyl | 1:100 | 0.2:20 |
| X.11 | Acibenzolar-S-methyl | 1:30 | 0.2:6 |
| X.12 | Pydiflumetofen | 1:3 | 2:6 |
| X.12 | Pydiflumetofen | 3.3:1 | 2:0.6 |
| X.12 | Pydiflumetofen | 1:30 | 0.2:6 |
| X.12 | Pydiflumetofen | 1:3 | 0.2:0.6 |
| X.12 | Benzovindiflupyr | 1:1 | 2:2 |
| X.12 | Benzovindiflupyr | 10:1 | 2:0.2 |
| X.12 | Benzovindiflupyr | 1:10 | 0.2:2 |
| X.12 | Benzovindiflupyr | 1:1 | 0.2:0.2 |
| X.12 | Cyprodinil | 1:10 | 2:20 |
| X.12 | Cyprodinil | 20:1 | 2:0.1 |
| X.12 | Cyprodinil | 1:100 | 0.2:20 |
| X.12 | Cyprodinil | 2:1 | 0.2:0.1 |
| X.12 | Fludioxonil | 1:3 | 2:6 |
| X.12 | Fludioxonil | 33.3:1 | 2:0.06 |
| X.12 | Fludioxonil | 1:30 | 0.2:6 |
| X.12 | Fludioxonil | 3.3:1 | 0.2:0.06 |
| X.12 | Azoxystrobin | 1:3 | 2:6 |
| X.12 | Azoxystrobin | 3.3:1 | 2:0.6 |
| X.12 | Azoxystrobin | 1:30 | 0.2:6 |
| X.12 | Azoxystrobin | 1:3 | 0.2:0.6 |
| X.12 | Difenoconazole | 1:3 | 2:6 |
| X.12 | Difenoconazole | 3.3:1 | 2:0.6 |
| X.12 | Difenoconazole | 1:30 | 0.2:6 |
| X.12 | Difenoconazole | 1:3 | 0.2:0.6 |
| X.12 | Prothioconazole | 3.3:1 | 2:0.6 |
| X.12 | Prothioconazole | 33.3:1 | 2:0.06 |
| X.12 | Prothioconazole | 1:3 | 0.2:0.6 |
| X.12 | Prothioconazole | 3.3:1 | 0.2:0.06 |
| X.12 | Mefentrifluconazole | 1:3 | 2:6 |
| X.12 | Mefentrifluconazole | 20:1 | 2:0.1 |
| X.12 | Mefentrifluconazole | 1:30 | 0.2:6 |
| X.12 | Mefentrifluconazole | 2:1 | 0.2:0.1 |
| X.12 | Florylpicoxamid | 2:1 | 2:1 |
| X.12 | Florylpicoxamid | 20:1 | 2:0.1 |
| X.12 | Florylpicoxamid | 1:5 | 0.2:1 |
| X.12 | Florylpicoxamid | 2:1 | 0.2:0.1 |
| X.12 | Acibenzolar-S-methyl | 1:10 | 2:20 |
| X.12 | Acibenzolar-S-methyl | 1:3 | 2:6 |
| X.12 | Acibenzolar-S-methyl | 1:100 | 0.2:20 |
| X.12 | Acibenzolar-S-methyl | 1:30 | 0.2:6 |
| X.13 | Pydiflumetofen | 1:30 | 0.2:6 |
| X.13 | Pydiflumetofen | 1:3 | 0.2:0.6 |
| X.13 | Pydiflumetofen | 1:300 | 0.02:6 |
| X.13 | Pydiflumetofen | 1:30 | 0.02:0.6 |
| X.13 | Benzovindiflupyr | 1:10 | 0.2:2 |
| X.13 | Benzovindiflupyr | 1:1 | 0.2:0.2 |
| X.13 | Benzovindiflupyr | 1:100 | 0.02:2 |
| X.13 | Benzovindiflupyr | 1:10 | 0.02:0.2 |
| X.13 | Cyprodinil | 1:100 | 0.2:20 |
| X.13 | Cyprodinil | 2:1 | 0.2:0.1 |
| X.13 | Cyprodinil | 1:1000 | 0.02:20 |
| X.13 | Cyprodinil | 1:5 | 0.02:0.1 |
| X.13 | Fludioxonil | 1:30 | 0.2:6 |
| X.13 | Fludioxonil | 3.3:1 | 0.2:0.06 |
| X.13 | Fludioxonil | 1:300 | 0.02:6 |
| X.13 | Fludioxonil | 1:3 | 0.02:0.06 |
| X.13 | Azoxystrobin | 1:30 | 0.2:6 |
| X.13 | Azoxystrobin | 1:3 | 0.2:0.6 |
| X.13 | Azoxystrobin | 1:300 | 0.02:6 |
| X.13 | Azoxystrobin | 1:30 | 0.02:0.6 |
| X.13 | Difenoconazole | 1:30 | 0.2:6 |
| X.13 | Difenoconazole | 1:3 | 0.2:0.6 |
| X.13 | Difenoconazole | 1:300 | 0.02:6 |
| X.13 | Difenoconazole | 1:30 | 0.02:0.6 |
| X.13 | Prothioconazole | 1:3 | 0.2:0.6 |
| X.13 | Prothioconazole | 3.3:1 | 0.2:0.06 |
| X.13 | Prothioconazole | 1:30 | 0.02:0.6 |
| X.13 | Prothioconazole | 1:3 | 0.02:0.06 |
| X.13 | Mefentrifluconazole | 1:30 | 0.2:6 |
| X.13 | Mefentrifluconazole | 2:1 | 0.2:0.1 |
| X.13 | Mefentrifluconazole | 1:300 | 0.02:6 |
| X.13 | Mefentrifluconazole | 1:5 | 0.02:0.1 |
| X.13 | Florylpicoxamid | 1:5 | 0.2:1 |
| X.13 | Florylpicoxamid | 2:1 | 0.2:0.1 |
| X.13 | Florylpicoxamid | 1:50 | 0.02:1 |
| X.13 | Florylpicoxamid | 1:5 | 0.02:0.1 |
| X.13 | Acibenzolar-S-methyl | 1:100 | 0.2:20 |
| X.13 | Acibenzolar-S-methyl | 1:30 | 0.2:6 |
| X.13 | Acibenzolar-S-methyl | 1:1000 | 0.02:20 |
| X.13 | Acibenzolar-S-methyl | 1:300 | 0.02:6 |
| X.15 | Pydiflumetofen | 1:3 | 2:6 |
| X.15 | Pydiflumetofen | 3.3:1 | 2:0.6 |
| X.15 | Pydiflumetofen | 1:10 | 0.6:6 |
| X.15 | Pydiflumetofen | 1:1 | 0.6:0.6 |
| X.15 | Benzovindiflupyr | 1:1 | 2:2 |
| X.15 | Benzovindiflupyr | 10:1 | 2:0.2 |
| X.15 | Benzovindiflupyr | 1:3.3 | 0.6:2 |
| X.15 | Benzovindiflupyr | 3:1 | 0.6:0.2 |
| X.15 | Cyprodinil | 1:10 | 2:20 |
| X.15 | Cyprodinil | 20:1 | 2:0.1 |
| X.15 | Cyprodinil | 1:33.3 | 0.6:20 |
| X.15 | Cyprodinil | 6:1 | 0.6:0.1 |
| X.15 | Fludioxonil | 1:3 | 2:6 |
| X.15 | Fludioxonil | 33.3:1 | 2:0.06 |
| X.15 | Fludioxonil | 1:10 | 0.6:6 |
| X.15 | Fludioxonil | 10:1 | 0.6:0.06 |
| X.15 | Azoxystrobin | 1:3 | 2:6 |
| X.15 | Azoxystrobin | 3.3:1 | 2:0.6 |
| X.15 | Azoxystrobin | 1:10 | 0.6:6 |
| X.15 | Azoxystrobin | 1:1 | 0.6:0.6 |
| X.15 | Difenoconazole | 1:3 | 2:6 |
| X.15 | Difenoconazole | 3.3:1 | 2:0.6 |
| X.15 | Difenoconazole | 1:10 | 0.6:6 |
| X.15 | Difenoconazole | 1:1 | 0.6:0.6 |
| X.15 | Prothioconazole | 3.3:1 | 2:0.6 |
| X.15 | Prothioconazole | 33.3:1 | 2:0.06 |
| X.15 | Prothioconazole | 1:1 | 0.6:0.6 |
| X.15 | Prothioconazole | 10:1 | 0.6:0.06 |
| X.15 | Mefentrifluconazole | 1:3 | 2:6 |
| X.15 | Mefentrifluconazole | 20:1 | 2:0.1 |
| X.15 | Mefentrifluconazole | 1:10 | 0.6:6 |
| X.15 | Mefentrifluconazole | 6:1 | 0.6:0.1 |
| X.15 | Florylpicoxamid | 2:1 | 2:1 |
| X.15 | Florylpicoxamid | 20:1 | 2:0.1 |
| X.15 | Florylpicoxamid | 1:1.6 | 0.6:1 |
| X.15 | Florylpicoxamid | 6:1 | 0.6:0.1 |
| X.15 | Acibenzolar-S-methyl | 1:10 | 2:20 |
| X.15 | Acibenzolar-S-methyl | 1:3 | 2:6 |
| X.15 | Acibenzolar-S-methyl | 1:33.3 | 0.6:20 |
| X.15 | Acibenzolar-S-methyl | 1:10 | 0.6:6 |
| X.16 | Pydiflumetofen | 1:1 | 6:6 |
| X.16 | Pydiflumetofen | 10:1 | 6:0.6 |
| X.16 | Pydiflumetofen | 1:6 | 1:6 |
| X.16 | Pydiflumetofen | 1.6:1 | 1:0.6 |
| X.16 | Benzovindiflupyr | 3:1 | 6:2 |
| X.16 | Benzovindiflupyr | 30:1 | 6:0.2 |
| X.16 | Benzovindiflupyr | 1:2 | 1:2 |
| X.16 | Benzovindiflupyr | 5:1 | 1:0.2 |
| X.16 | Cyprodinil | 1:3.3 | 6:20 |
| X.16 | Cyprodinil | 60:1 | 6:0.1 |
| X.16 | Cyprodinil | 1:20 | 1:20 |
| X.16 | Cyprodinil | 10:1 | 1:0.1 |
| X.16 | Fludioxonil | 1:1 | 6:6 |
| X.16 | Fludioxonil | 100:1 | 6:0.06 |
| X.16 | Fludioxonil | 1:6 | 1:6 |
| X.16 | Azoxystrobin | 1:1 | 6:6 |
| X.16 | Azoxystrobin | 10:1 | 6:0.6 |
| X.16 | Azoxystrobin | 1:6 | 1:6 |
| X.16 | Azoxystrobin | 1.6:1 | 1:0.6 |
| X.16 | Difenoconazole | 1:1 | 6:6 |
| X.16 | Difenoconazole | 10:1 | 6:0.6 |
| X.16 | Difenoconazole | 1:6 | 1:6 |
| X.16 | Prothioconazole | 10:1 | 6:0.6 |
| X.16 | Prothioconazole | 100:1 | 6:0.06 |
| X.16 | Mefentrifluconazole | 1:1 | 6:6 |
| X.16 | Mefentrifluconazole | 60:1 | 6:0.1 |
| X.16 | Florylpicoxamid | 6:1 | 6:1 |
| X.16 | Florylpicoxamid | 60:1 | 6:0.1 |
| X.16 | Florylpicoxamid | 1:1 | 1:1 |
| X.16 | Acibenzolar-S-methyl | 1:3.3 | 6:20 |
| X.16 | Acibenzolar-S-methyl | 1:1 | 6:6 |
| X.14 | Pydiflumetofen | 3.3:1 | 20:6 |
| X.14 | Pydiflumetofen | 33.3:1 | 20:0.6 |
| X.14 | Pydiflumetofen | 1:1 | 6:6 |
| X.14 | Pydiflumetofen | 10:1 | 6:0.6 |
| X.14 | Benzovindiflupyr | 10:1 | 20:2 |
| X.14 | Benzovindiflupyr | 100:1 | 20:0.2 |
| X.14 | Benzovindiflupyr | 3:1 | 6:2 |
| X.14 | Benzovindiflupyr | 30:1 | 6:0.2 |
| X.14 | Cyprodinil | 1:1 | 20:20 |
| X.14 | Cyprodinil | 200:1 | 20:0.1 |
| X.14 | Cyprodinil | 1:3.3 | 6:20 |
| X.14 | Cyprodinil | 60:1 | 6:0.1 |
| X.14 | Fludioxonil | 3.3:1 | 20:6 |
| X.14 | Fludioxonil | 333.3:1 | 20:0.06 |
| X.14 | Fludioxonil | 1:1 | 6:6 |
| X.14 | Fludioxonil | 100:1 | 6:0.06 |
| X.14 | Azoxystrobin | 3.3:1 | 20:6 |
| X.14 | Azoxystrobin | 33.3:1 | 20:0.6 |
| X.14 | Azoxystrobin | 1:1 | 6:6 |
| X.14 | Azoxystrobin | 10:1 | 6:0.6 |
| X.14 | Difenoconazole | 3.3:1 | 20:6 |
| X.14 | Difenoconazole | 33.3:1 | 20:0.6 |
| X.14 | Difenoconazole | 1:1 | 6:6 |
| X.14 | Difenoconazole | 10:1 | 6:0.6 |
| X.14 | Prothioconazole | 33.3:1 | 20:0.6 |
| X.14 | Prothioconazole | 333.3:1 | 20:0.06 |
| X.14 | Prothioconazole | 10:1 | 6:0.6 |
| X.14 | Prothioconazole | 100:1 | 6:0.06 |
| X.14 | Mefentrifluconazole | 3.3:1 | 20:6 |
| X.14 | Mefentrifluconazole | 200:1 | 20:0.1 |
| X.14 | Mefentrifluconazole | 1:1 | 6:6 |
| X.14 | Mefentrifluconazole | 60:1 | 6:0.1 |
| X.14 | Florylpicoxamid | 20:1 | 20:1 |
| X.14 | Florylpicoxamid | 200:1 | 20:0.1 |
| X.14 | Florylpicoxamid | 6:1 | 6:1 |
| X.14 | Florylpicoxamid | 60:1 | 6:0.1 |
| X.14 | Acibenzolar-S-methyl | 1:1 | 20:20 |
| X.14 | Acibenzolar-S-methyl | 3.3:1 | 20:6 |
| X.14 | Acibenzolar-S-methyl | 1:3.3 | 6:20 |
| X.14 | Acibenzolar-S-methyl | 1:1 | 6:6 |

### Example M-B3: Septoria tritici (Leaf Blotch)

Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically and visually after 72 hrs.

The following mixture compositions (A:B) at the reported concentration (in ppm) gave at least 70% disease control in the test system. Comp. A means Component A

| | | | Conc. |
|---|---|---|---|
| Comp. | | Ratio | (ppm) |
| A | Component B | A:B | (A:B) |
| X.01 | Pydiflumetofen | 1:30 | 0.2:6 |
| X.01 | Pydiflumetofen | 1:3 | 0.2:0.6 |
| X.01 | Pydiflumetofen | 1:100 | 0.06:6 |
| X.01 | Pydiflumetofen | 1:10 | 0.06:0.6 |
| X.01 | Benzovindiflupyr | 1:10 | 0.2:2 |
| X.01 | Benzovindiflupyr | 1:1 | 0.2:0.2 |
| X.01 | Benzovindiflupyr | 1:33.3 | 0.06:2 |
| X.01 | Benzovindiflupyr | 1:3.3 | 0.06:0.2 |
| X.01 | Cyprodinil | 1:100 | 0.2:20 |
| X.01 | Cyprodinil | 2:1 | 0.2:0.1 |
| X.01 | Cyprodinil | 1:333.3 | 0.06:20 |
| X.01 | Cyprodinil | 1:1.6 | 0.06:0.1 |
| X.01 | Fludioxonil | 1:30 | 0.2:6 |
| X.01 | Fludioxonil | 3.3:1 | 0.2:0.06 |
| X.01 | Fludioxonil | 1:100 | 0.06:6 |
| X.01 | Fludioxonil | 1:1 | 0.06:0.06 |
| X.01 | Azoxystrobin | 1:30 | 0.2:6 |
| X.01 | Azoxystrobin | 1:3 | 0.2:0.6 |
| X.01 | Azoxystrobin | 1:100 | 0.06:6 |
| X.01 | Azoxystrobin | 1:10 | 0.06:0.6 |
| X.01 | Difenoconazole | 1:30 | 0.2:6 |
| X.01 | Difenoconazole | 1:3 | 0.2:0.6 |
| X.01 | Difenoconazole | 1:100 | 0.06:6 |
| X.01 | Difenoconazole | 1:10 | 0.06:0.6 |
| X.01 | Prothioconazole | 1:3 | 0.2:0.6 |
| X.01 | Prothioconazole | 3.3:1 | 0.2:0.06 |
| X.01 | Prothioconazole | 1:10 | 0.06:0.6 |
| X.01 | Prothioconazole | 1:1 | 0.06:0.06 |
| X.01 | Mefentrifluconazole | 1:30 | 0.2:6 |
| X.01 | Mefentrifluconazole | 2:1 | 0.2:0.1 |
| X.01 | Mefentrifluconazole | 1:100 | 0.06:6 |
| X.01 | Mefentrifluconazole | 1:1.6 | 0.06:0.1 |
| X.01 | Florylpicoxamid | 1:5 | 0.2:1 |
| X.01 | Florylpicoxamid | 2:1 | 0.2:0.1 |
| X.01 | Florylpicoxamid | 1:16.6 | 0.06:1 |
| X.01 | Florylpicoxamid | 1:1.6 | 0.06:0.1 |
| X.01 | Acibenzolar-S-methyl | 1:100 | 0.2:20 |
| X.01 | Acibenzolar-S-methyl | 1:30 | 0.2:6 |
| X.01 | Acibenzolar-S-methyl | 1:333.3 | 0.06:20 |
| X.01 | Acibenzolar-S-methyl | 1:100 | 0.06:6 |
| X.05 | Pydiflumetofen | 1:30 | 0.2:6 |
| X.05 | Pydiflumetofen | 1:3 | 0.2:0.6 |
| X.05 | Pydiflumetofen | 1:100 | 0.06:6 |
| X.05 | Pydiflumetofen | 1:10 | 0.06:0.6 |
| X.05 | Benzovindiflupyr | 1:10 | 0.2:2 |
| X.05 | Benzovindiflupyr | 1:1 | 0.2:0.2 |
| X.05 | Benzovindiflupyr | 1:33.3 | 0.06:2 |
| X.05 | Benzovindiflupyr | 1:3.3 | 0.06:0.2 |
| X.05 | Cyprodinil | 1:100 | 0.2:20 |
| X.05 | Cyprodinil | 2:1 | 0.2:0.1 |
| X.05 | Cyprodinil | 1:333.3 | 0.06:20 |
| X.05 | Cyprodinil | 1:1.6 | 0.06:0.1 |
| X.05 | Fludioxonil | 1:30 | 0.2:6 |
| X.05 | Fludioxonil | 3.3:1 | 0.2:0.06 |
| X.05 | Fludioxonil | 1:100 | 0.06:6 |
| X.05 | Fludioxonil | 1:1 | 0.06:0.06 |
| X.05 | Azoxystrobin | 1:30 | 0.2:6 |
| X.05 | Azoxystrobin | 1:3 | 0.2:0.6 |
| X.05 | Azoxystrobin | 1:100 | 0.06:6 |
| X.05 | Azoxystrobin | 1:10 | 0.06:0.6 |
| X.05 | Difenoconazole | 1:30 | 0.2:6 |
| X.05 | Difenoconazole | 1:3 | 0.2:0.6 |
| X.05 | Difenoconazole | 1:100 | 0.06:6 |
| X.05 | Difenoconazole | 1:10 | 0.06:0.6 |
| X.05 | Prothioconazole | 1:3 | 0.2:0.6 |
| X.05 | Prothioconazole | 3.3:1 | 0.2:0.06 |
| X.05 | Prothioconazole | 1:10 | 0.06:0.6 |
| X.05 | Prothioconazole | 1:1 | 0.06:0.06 |
| X.05 | Mefentrifluconazole | 1:30 | 0.2:6 |
| X.05 | Mefentrifluconazole | 2:1 | 0.2:0.1 |
| X.05 | Mefentrifluconazole | 1:100 | 0.06:6 |
| X.05 | Mefentrifluconazole | 1:1.6 | 0.06:0.1 |
| X.05 | Florylpicoxamid | 1:5 | 0.2:1 |
| X.05 | Florylpicoxamid | 2:1 | 0.2:0.1 |
| X.05 | Florylpicoxamid | 1:16.6 | 0.06:1 |
| X.05 | Florylpicoxamid | 1:1.6 | 0.06:0.1 |
| X.05 | Acibenzolar-S-methyl | 1:100 | 0.2:20 |
| X.05 | Acibenzolar-S-methyl | 1:30 | 0.2:6 |
| X.05 | Acibenzolar-S-methyl | 1:333.3 | 0.06:20 |
| X.05 | Acibenzolar-S-methyl | 1:100 | 0.06:6 |
| X.03 | Pydiflumetofen | 1:1 | 6:6 |
| X.03 | Pydiflumetofen | 10:1 | 6:0.6 |
| X.03 | Pydiflumetofen | 1:6 | 1:6 |
| X.03 | Pydiflumetofen | 1.6:1 | 1:0.6 |
| X.03 | Benzovindiflupyr | 3:1 | 6:2 |
| X.03 | Benzovindiflupyr | 30:1 | 6:0.2 |
| X.03 | Benzovindiflupyr | 1:2 | 1:2 |
| X.03 | Benzovindiflupyr | 5:1 | 1:0.2 |
| X.03 | Cyprodinil | 1:3.3 | 6:20 |
| X.03 | Cyprodinil | 60:1 | 6:0.1 |
| X.03 | Cyprodinil | 1:20 | 1:20 |
| X.03 | Cyprodinil | 10:1 | 1:0.1 |
| X.03 | Fludioxonil | 1:1 | 6:6 |
| X.03 | Fludioxonil | 100:1 | 6:0.06 |
| X.03 | Fludioxonil | 1:6 | 1:6 |
| X.03 | Fludioxonil | 16.6:1 | 1:0.6 |
| X.03 | Azoxystrobin | 1:1 | 6:6 |
| X.03 | Azoxystrobin | 10:1 | 6:0.6 |
| X.03 | Azoxystrobin | 1:6 | 1:6 |
| X.03 | Azoxystrobin | 1.6:1 | 1:0.6 |
| X.03 | Difenoconazole | 1:1 | 6:6 |
| X.03 | Difenoconazole | 10:1 | 6:0.6 |
| X.03 | Difenoconazole | 1:6 | 1:6 |
| X.03 | Difenoconazole | 1.6:1 | 1:0.6 |
| X.03 | Prothioconazole | 10:1 | 6:0.6 |
| X.03 | Prothioconazole | 100:1 | 6:0.06 |
| X.03 | Prothioconazole | 1.6:1 | 1:0.6 |
| X.03 | Prothioconazole | 16.6:1 | 1:0.6 |
| X.03 | Mefentrifluconazole | 1:1 | 6:6 |
| X.03 | Mefentrifluconazole | 60:1 | 6:0.1 |
| X.03 | Mefentrifluconazole | 1:6 | 1:6 |
| X.03 | Mefentrifluconazole | 10:1 | 1:0.1 |
| X.03 | Florylpicoxamid | 6:1 | 6:1 |
| X.03 | Florylpicoxamid | 60:1 | 6:0.1 |
| X.03 | Florylpicoxamid | 1:1 | 1:1 |
| X.03 | Florylpicoxamid | 10:1 | 1:0.1 |
| X.03 | Acibenzolar-S-methyl | 1:3.3 | 6:20 |
| X.03 | Acibenzolar-S-methyl | 1:1 | 6:6 |
| X.03 | Acibenzolar-S-methyl | 1:20 | 1:20 |
| X.03 | Acibenzolar-S-methyl | 1:6 | 1:6 |
| X.08 | Pydiflumetofen | 1:1 | 6:6 |
| X.08 | Pydiflumetofen | 10:1 | 6:0.6 |
| X.08 | Pydiflumetofen | 1:6 | 1:6 |
| X.08 | Pydiflumetofen | 1.6:1 | 1:0.6 |
| X.08 | Benzovindiflupyr | 3:1 | 6:2 |
| X.08 | Benzovindiflupyr | 30:1 | 6:0.2 |
| X.08 | Benzovindiflupyr | 1:2 | 1:2 |
| X.08 | Benzovindiflupyr | 5:1 | 1:0.2 |
| X.08 | Cyprodinil | 1:3.3 | 6:20 |
| X.08 | Cyprodinil | 60:1 | 6:0.1 |
| X.08 | Cyprodinil | 1:20 | 1:20 |
| X.08 | Cyprodinil | 10:1 | 1:0.1 |
| X.08 | Fludioxonil | 1:1 | 6:6 |
| X.08 | Fludioxonil | 100:1 | 6:0.06 |
| X.08 | Fludioxonil | 1:6 | 1:6 |
| X.08 | Fludioxonil | 16.6:1 | 1:0.6 |
| X.08 | Azoxystrobin | 1:1 | 6:6 |
| X.08 | Azoxystrobin | 10:1 | 6:0.6 |
| X.08 | Azoxystrobin | 1:6 | 1:6 |
| X.08 | Azoxystrobin | 1.6:1 | 1:0.6 |
| X.08 | Difenoconazole | 1:1 | 6:6 |
| X.08 | Difenoconazole | 10:1 | 6:0.6 |
| X.08 | Difenoconazole | 1:6 | 1:6 |
| X.08 | Difenoconazole | 1.6:1 | 1:0.6 |
| X.08 | Prothioconazole | 10:1 | 6:0.6 |
| X.08 | Prothioconazole | 100:1 | 6:0.06 |
| X.08 | Prothioconazole | 1.6:1 | 1:0.6 |
| X.08 | Prothioconazole | 16.6:1 | 1:0.6 |
| X.08 | Mefentrifluconazole | 1:1 | 6:6 |
| X.08 | Mefentrifluconazole | 60:1 | 6:0.1 |
| X.08 | Mefentrifluconazole | 1:6 | 1:6 |
| X.08 | Mefentrifluconazole | 10:1 | 1:0.1 |
| X.08 | Florylpicoxamid | 6:1 | 6:1 |
| X.08 | Florylpicoxamid | 60:1 | 6:0.1 |
| X.08 | Florylpicoxamid | 1:1 | 1:1 |
| X.08 | Florylpicoxamid | 10:1 | 1:0.1 |
| X.08 | Acibenzolar-S-methyl | 1:3.3 | 6:20 |
| X.08 | Acibenzolar-S-methyl | 1:1 | 6:6 |
| X.08 | Acibenzolar-S-methyl | 1:20 | 1:20 |
| X.08 | Acibenzolar-S-methyl | 1:6 | 1:6 |
| X.10 | Pydiflumetofen | 1:1 | 6:6 |
| X.10 | Pydiflumetofen | 10:1 | 6:0.6 |
| X.10 | Pydiflumetofen | 1:6 | 1:6 |
| X.10 | Pydiflumetofen | 1.6:1 | 1:0.6 |
| X.10 | Benzovindiflupyr | 3:1 | 6:2 |
| X.10 | Benzovindiflupyr | 30:1 | 6:0.2 |
| X.10 | Benzovindiflupyr | 1:2 | 1:2 |
| X.10 | Benzovindiflupyr | 5:1 | 1:0.2 |
| X.10 | Cyprodinil | 1:3.3 | 6:20 |
| X.10 | Cyprodinil | 60:1 | 6:0.1 |
| X.10 | Cyprodinil | 1:20 | 1:20 |
| X.10 | Cyprodinil | 10:1 | 1:0.1 |
| X.10 | Fludioxonil | 1:1 | 6:6 |
| X.10 | Fludioxonil | 100:1 | 6:0.06 |
| X.10 | Fludioxonil | 1:6 | 1:6 |
| X.10 | Fludioxonil | 16.6:1 | 1:0.6 |
| X.10 | Azoxystrobin | 1:1 | 6:6 |
| X.10 | Azoxystrobin | 10:1 | 6:0.6 |
| X.10 | Azoxystrobin | 1:6 | 1:6 |
| X.10 | Azoxystrobin | 1.6:1 | 1:0.6 |
| X.10 | Difenoconazole | 1:1 | 6:6 |
| X.10 | Difenoconazole | 10:1 | 6:0.6 |
| X.10 | Difenoconazole | 1:6 | 1:6 |
| X.10 | Difenoconazole | 1.6:1 | 1:0.6 |
| X.10 | Prothioconazole | 10:1 | 6:0.6 |
| X.10 | Prothioconazole | 100:1 | 6:0.06 |
| X.10 | Prothioconazole | 1.6:1 | 1:0.6 |
| X.10 | Prothioconazole | 16.6:1 | 1:0.6 |
| X.10 | Mefentrifluconazole | 1:1 | 6:6 |
| X.10 | Mefentrifluconazole | 60:1 | 6:0.1 |
| X.10 | Mefentrifluconazole | 1:6 | 1:6 |
| X.10 | Mefentrifluconazole | 10:1 | 1:0.1 |
| X.10 | Florylpicoxamid | 6:1 | 6:1 |
| X.10 | Florylpicoxamid | 60:1 | 6:0.1 |
| X.10 | Florylpicoxamid | 1:1 | 1:1 |
| X.10 | Florylpicoxamid | 10:1 | 1:0.1 |
| X.10 | Acibenzolar-S-methyl | 1:1 | 6:6 |
| X.10 | Acibenzolar-S-methyl | 1:20 | 1:20 |
| X.10 | Acibenzolar-S-methyl | 1:6 | 1:6 |
| X.02 | Pydiflumetofen | 1:3 | 2:6 |
| X.02 | Pydiflumetofen | 3.3:1 | 2:0.6 |
| X.02 | Pydiflumetofen | 1:10 | 0.6:6 |
| X.02 | Pydiflumetofen | 1:1 | 0.6:0.6 |
| X.02 | Benzovindiflupyr | 1:1 | 2:2 |
| X.02 | Benzovindiflupyr | 10:1 | 2:0.2 |
| X.02 | Benzovindiflupyr | 1:3.3 | 0.6:2 |
| X.02 | Benzovindiflupyr | 3:1 | 0.6:0.2 |
| X.02 | Cyprodinil | 1:10 | 2:20 |
| X.02 | Cyprodinil | 20:1 | 2:0.1 |
| X.02 | Cyprodinil | 1:33.3 | 0.6:20 |
| X.02 | Cyprodinil | 6:1 | 0.6:0.1 |
| X.02 | Fludioxonil | 1:3 | 2:6 |
| X.02 | Fludioxonil | 33.3:1 | 2:0.06 |
| X.02 | Fludioxonil | 1:10 | 0.6:6 |
| X.02 | Fludioxonil | 10:1 | 0.6:0.06 |
| X.02 | Azoxystrobin | 1:3 | 2:6 |
| X.02 | Azoxystrobin | 3.3:1 | 2:0.6 |
| X.02 | Azoxystrobin | 1:10 | 0.6:6 |
| X.02 | Azoxystrobin | 1:1 | 0.6:0.6 |
| X.02 | Difenoconazole | 1:3 | 2:6 |
| X.02 | Difenoconazole | 3.3:1 | 2:0.6 |
| X.02 | Difenoconazole | 1:10 | 0.6:6 |
| X.02 | Difenoconazole | 1:1 | 0.6:0.6 |
| X.02 | Prothioconazole | 3.3:1 | 2:0.6 |
| X.02 | Prothioconazole | 33.3:1 | 2:0.06 |
| X.02 | Prothioconazole | 1:1 | 0.6:0.6 |
| X.02 | Prothioconazole | 10:1 | 0.6:0.06 |
| X.02 | Mefentrifluconazole | 1:3 | 2:6 |
| X.02 | Mefentrifluconazole | 20:1 | 2:0.1 |
| X.02 | Mefentrifluconazole | 1:10 | 0.6:6 |
| X.02 | Mefentrifluconazole | 6:1 | 0.6:0.1 |
| X.02 | Florylpicoxamid | 2:1 | 2:1 |
| X.02 | Florylpicoxamid | 20:1 | 2:0.1 |
| X.02 | Florylpicoxamid | 1:1.6 | 0.6:1 |
| X.02 | Florylpicoxamid | 6:1 | 0.6:0.1 |
| X.02 | Acibenzolar-S-methyl | 1:10 | 2:20 |
| X.02 | Acibenzolar-S-methyl | 1:3 | 2:6 |
| X.02 | Acibenzolar-S-methyl | 1:33.3 | 0.6:20 |
| X.02 | Acibenzolar-S-methyl | 1:10 | 0.6:6 |
| X.04 | Pydiflumetofen | 1:1 | 6:6 |
| X.04 | Pydiflumetofen | 10:1 | 6:0.6 |
| X.04 | Pydiflumetofen | 1:10 | 0.6:6 |
| X.04 | Pydiflumetofen | 1:1 | 0.6:0.6 |
| X.04 | Benzovindiflupyr | 3:1 | 6:2 |
| X.04 | Benzovindiflupyr | 1:3.3 | 0.6:2 |
| X.04 | Benzovindiflupyr | 3:1 | 0.6:0.2 |
| X.04 | Cyprodinil | 1:3.3 | 6:20 |
| X.04 | Cyprodinil | 1:33.3 | 0.6:20 |
| X.04 | Cyprodinil | 6:1 | 0.6:0.1 |
| X.04 | Fludioxonil | 1:1 | 6:6 |
| X.04 | Fludioxonil | 100:1 | 6:0.06 |
| X.04 | Fludioxonil | 1:10 | 0.6:6 |
| X.04 | Fludioxonil | 10:1 | 0.6:0.06 |
| X.04 | Azoxystrobin | 1:1 | 6:6 |
| X.04 | Azoxystrobin | 1:10 | 0.6:6 |
| X.04 | Azoxystrobin | 1:1 | 0.6:0.6 |
| X.04 | Difenoconazole | 1:1 | 6:6 |
| X.04 | Difenoconazole | 10:1 | 6:0.6 |
| X.04 | Difenoconazole | 1:10 | 0.6:6 |
| X.04 | Difenoconazole | 1:1 | 0.6:0.6 |
| X.04 | Prothioconazole | 1:1 | 0.6:0.6 |
| X.04 | Prothioconazole | 10:1 | 0.6:0.06 |
| X.04 | Mefentrifluconazole | 1:1 | 6:6 |
| X.04 | Mefentrifluconazole | 60:1 | 6:0.1 |
| X.04 | Mefentrifluconazole | 1:10 | 0.6:6 |
| X.04 | Mefentrifluconazole | 6:1 | 0.6:0.1 |
| X.04 | Florylpicoxamid | 6:1 | 6:1 |
| X.04 | Florylpicoxamid | 60:1 | 6:0.1 |
| X.04 | Florylpicoxamid | 1:1.6 | 0.6:1 |
| X.04 | Florylpicoxamid | 6:1 | 0.6:1 |
| X.04 | Acibenzolar-S-methyl | 1:33.3 | 0.6:20 |
| X.04 | Acibenzolar-S-methyl | 1:10 | 0.6:6 |
| X.06 | Pydiflumetofen | 3.3:1 | 20:6 |
| X.06 | Pydiflumetofen | 33.3:1 | 20:0.6 |
| X.06 | Pydiflumetofen | 1:6 | 1:6 |
| X.06 | Pydiflumetofen | 1.6:1 | 1:0.6 |
| X.06 | Benzovindiflupyr | 10:1 | 20:2 |
| X.06 | Benzovindiflupyr | 100:1 | 20:0.2 |
| X.06 | Benzovindiflupyr | 1:2 | 1:2 |
| X.06 | Benzovindiflupyr | 5:1 | 1:0.2 |
| X.06 | Cyprodinil | 1:1 | 20:20 |
| X.06 | Cyprodinil | 200:1 | 20:0.1 |
| X.06 | Cyprodinil | 1:20 | 1:20 |
| X.06 | Cyprodinil | 10:1 | 1:0.1 |
| X.06 | Fludioxonil | 3.3:1 | 20:6 |
| X.06 | Fludioxonil | 333.3:1 | 20:0.06 |
| X.06 | Fludioxonil | 1:6 | 1:6 |
| X.06 | Fludioxonil | 16.6:1 | 1:0.6 |
| X.06 | Azoxystrobin | 3.3:1 | 20:6 |
| X.06 | Azoxystrobin | 33.3:1 | 20:0.6 |
| X.06 | Azoxystrobin | 1:6 | 1:6 |
| X.06 | Azoxystrobin | 1.6:1 | 1:0.6 |
| X.06 | Difenoconazole | 3.3:1 | 20:6 |
| X.06 | Difenoconazole | 33.3:1 | 20:0.6 |
| X.06 | Difenoconazole | 1:6 | 1:6 |
| X.06 | Difenoconazole | 1.6:1 | 1:0.6 |
| X.06 | Prothioconazole | 33.3:1 | 20:0.6 |
| X.06 | Prothioconazole | 333.3:1 | 20:0.06 |
| X.06 | Prothioconazole | 1.6:1 | 1:0.6 |
| X.06 | Prothioconazole | 16.6:1 | 1:0.6 |
| X.06 | Mefentrifluconazole | 3.3:1 | 20:6 |
| X.06 | Mefentrifluconazole | 200:1 | 20:0.1 |
| X.06 | Mefentrifluconazole | 1:6 | 1:6 |
| X.06 | Mefentrifluconazole | 10:1 | 1:0.1 |
| X.06 | Florylpicoxamid | 20:1 | 20:1 |
| X.06 | Florylpicoxamid | 200:1 | 20:0.1 |
| X.06 | Florylpicoxamid | 1:1 | 1:1 |
| X.06 | Florylpicoxamid | 10:1 | 1:0.1 |
| X.06 | Acibenzolar-S-methyl | 1:1 | 20:20 |
| X.06 | Acibenzolar-S-methyl | 3.3:1 | 20:6 |
| X.06 | Acibenzolar-S-methyl | 1:20 | 1:20 |
| X.06 | Acibenzolar-S-methyl | 1:6 | 1:6 |
| X.07 | Pydiflumetofen | 1:6 | 1:6 |
| X.07 | Pydiflumetofen | 1.6:1 | 1:0.6 |
| X.07 | Pydiflumetofen | 1:60 | 0.1:6 |
| X.07 | Pydiflumetofen | 1:6 | 0.1:0.6 |
| X.07 | Benzovindiflupyr | 1:2 | 1:2 |
| X.07 | Benzovindiflupyr | 5:1 | 1:0.2 |
| X.07 | Benzovindiflupyr | 1:20 | 0.1:2 |
| X.07 | Benzovindiflupyr | 1:2 | 0.1:0.2 |
| X.07 | Cyprodinil | 1:20 | 1:20 |
| X.07 | Cyprodinil | 10:1 | 1:0.1 |
| X.07 | Cyprodinil | 1:200 | 0.1:20 |
| X.07 | Cyprodinil | 1:1 | 0.1:0.1 |
| X.07 | Fludioxonil | 1:6 | 1:6 |
| X.07 | Fludioxonil | 16.6:1 | 1:0.6 |
| X.07 | Fludioxonil | 1:60 | 0.1:6 |
| X.07 | Fludioxonil | 1.6:1 | 0.1:0.06 |
| X.07 | Azoxystrobin | 1:6 | 1:6 |
| X.07 | Azoxystrobin | 1.6:1 | 1:0.6 |
| X.07 | Azoxystrobin | 1:60 | 0.1:6 |
| X.07 | Azoxystrobin | 1:6 | 0.1:0.6 |
| X.07 | Difenoconazole | 1:6 | 1:6 |
| X.07 | Difenoconazole | 1.6:1 | 1:0.6 |
| X.07 | Difenoconazole | 1:60 | 0.1:6 |
| X.07 | Difenoconazole | 1:6 | 0.1:0.6 |
| X.07 | Prothioconazole | 1.6:1 | 1:0.6 |
| X.07 | Prothioconazole | 16.6:1 | 1:0.6 |
| X.07 | Prothioconazole | 1:6 | 0.1:0.6 |
| X.07 | Prothioconazole | 1.6:1 | 0.1:0.06 |
| X.07 | Mefentrifluconazole | 1:6 | 1:6 |
| X.07 | Mefentrifluconazole | 10:1 | 1:0.1 |
| X.07 | Mefentrifluconazole | 1:60 | 0.1:6 |
| X.07 | Mefentrifluconazole | 1:1 | 0.1:0.1 |
| X.07 | Florylpicoxamid | 1:1 | 1:1 |
| X.07 | Florylpicoxamid | 10:1 | 1:0.1 |
| X.07 | Florylpicoxamid | 1:10 | 0.1:1 |
| X.07 | Florylpicoxamid | 1:1 | 0.1:0.1 |
| X.07 | Acibenzolar-S-methyl | 1:20 | 1:20 |
| X.07 | Acibenzolar-S-methyl | 1:6 | 1:6 |
| X.07 | Acibenzolar-S-methyl | 1:200 | 0.1:20 |
| X.09 | Pydiflumetofen | 3.3:1 | 20:6 |
| X.09 | Pydiflumetofen | 33.3:1 | 20:0.6 |
| X.09 | Pydiflumetofen | 1:3 | 2:6 |
| X.09 | Pydiflumetofen | 3.3:1 | 2:0.6 |
| X.09 | Benzovindiflupyr | 10:1 | 20:2 |
| X.09 | Benzovindiflupyr | 100:1 | 20:0.2 |
| X.09 | Benzovindiflupyr | 1:1 | 2:2 |
| X.09 | Benzovindiflupyr | 10:1 | 2:0.2 |
| X.09 | Cyprodinil | 1:1 | 20:20 |
| X.09 | Cyprodinil | 1:10 | 2:20 |
| X.09 | Fludioxonil | 3.3:1 | 20:6 |
| X.09 | Fludioxonil | 1:3 | 2:6 |
| X.09 | Azoxystrobin | 3.3:1 | 20:6 |
| X.09 | Azoxystrobin | 1:3 | 2:6 |
| X.09 | Difenoconazole | 3.3:1 | 20:6 |
| X.09 | Difenoconazole | 33.3:1 | 20:0.6 |
| X.09 | Difenoconazole | 3.3:1 | 2:0.6 |
| X.09 | Mefentrifluconazole | 3.3:1 | 20:6 |
| X.09 | Mefentrifluconazole | 200:1 | 20:0.1 |
| X.09 | Mefentrifluconazole | 1:3 | 2:6 |
| X.09 | Mefentrifluconazole | 20:1 | 2:0.1 |
| X.09 | Florylpicoxamid | 20:1 | 20:1 |
| X.09 | Florylpicoxamid | 200:1 | 20:0.1 |
| X.09 | Florylpicoxamid | 2:1 | 2:1 |
| X.09 | Florylpicoxamid | 20:1 | 2:0.1 |
| X.09 | Acibenzolar-S-methyl | 1:1 | 20:20 |
| X.09 | Acibenzolar-S-methyl | 1:10 | 2:20 |
| X.11 | Pydiflumetofen | 1:3 | 2:6 |
| X.11 | Pydiflumetofen | 3.3:1 | 2:0.6 |
| X.11 | Pydiflumetofen | 1:30 | 0.2:6 |
| X.11 | Pydiflumetofen | 1:3 | 0.2:0.6 |
| X.11 | Benzovindiflupyr | 1:1 | 2:2 |
| X.11 | Benzovindiflupyr | 10:1 | 2:0.2 |
| X.11 | Benzovindiflupyr | 1:10 | 0.2:2 |
| X.11 | Benzovindiflupyr | 1:1 | 0.2:0.2 |
| X.11 | Cyprodinil | 1:10 | 2:20 |
| X.11 | Cyprodinil | 20:1 | 2:0.1 |
| X.11 | Cyprodinil | 1:100 | 0.2:20 |
| X.11 | Cyprodinil | 2:1 | 0.2:0.1 |
| X.11 | Fludioxonil | 1:3 | 2:6 |
| X.11 | Fludioxonil | 33.3:1 | 2:0.06 |
| X.11 | Fludioxonil | 1:30 | 0.2:6 |
| X.11 | Fludioxonil | 3.3:1 | 0.2:0.06 |
| X.11 | Azoxystrobin | 1:3 | 2:6 |
| X.11 | Azoxystrobin | 3.3:1 | 2:0.6 |
| X.11 | Azoxystrobin | 1:30 | 0.2:6 |
| X.11 | Azoxystrobin | 1:3 | 0.2:0.6 |
| X.11 | Difenoconazole | 1:3 | 2:6 |
| X.11 | Difenoconazole | 3.3:1 | 2:0.6 |
| X.11 | Difenoconazole | 1:30 | 0.2:6 |
| X.11 | Difenoconazole | 1:3 | 0.2:0.6 |
| X.11 | Prothioconazole | 3.3:1 | 2:0.6 |
| X.11 | Prothioconazole | 33.3:1 | 2:0.06 |
| X.11 | Prothioconazole | 1:3 | 0.2:0.6 |
| X.11 | Prothioconazole | 3.3:1 | 0.2:0.06 |
| X.11 | Mefentrifluconazole | 1:3 | 2:6 |
| X.11 | Mefentrifluconazole | 20:1 | 2:0.1 |
| X.11 | Mefentrifluconazole | 1:30 | 0.2:6 |
| X.11 | Mefentrifluconazole | 2:1 | 0.2:0.1 |
| X.11 | Florylpicoxamid | 2:1 | 2:1 |
| X.11 | Florylpicoxamid | 20:1 | 2:0.1 |
| X.11 | Florylpicoxamid | 1:5 | 0.2:1 |
| X.11 | Florylpicoxamid | 2:1 | 0.2:0.1 |
| X.11 | Acibenzolar-S-methyl | 1:10 | 2:20 |
| X.11 | Acibenzolar-S-methyl | 1:3 | 2:6 |
| X.11 | Acibenzolar-S-methyl | 1:100 | 0.2:20 |
| X.11 | Acibenzolar-S-methyl | 1:30 | 0.2:6 |
| X.12 | Pydiflumetofen | 1:3 | 2:6 |
| X.12 | Pydiflumetofen | 3.3:1 | 2:0.6 |
| X.12 | Pydiflumetofen | 1:30 | 0.2:6 |
| X.12 | Pydiflumetofen | 1:3 | 0.2:0.6 |
| X.12 | Benzovindiflupyr | 1:1 | 2:2 |
| X.12 | Benzovindiflupyr | 10:1 | 2:0.2 |
| X.12 | Benzovindiflupyr | 1:10 | 0.2:2 |
| X.12 | Benzovindiflupyr | 1:1 | 0.2:0.2 |
| X.12 | Cyprodinil | 1:10 | 2:20 |
| X.12 | Cyprodinil | 20:1 | 2:0.1 |
| X.12 | Cyprodinil | 1:100 | 0.2:20 |
| X.12 | Cyprodinil | 2:1 | 0.2:0.1 |
| X.12 | Fludioxonil | 1:3 | 2:6 |
| X.12 | Fludioxonil | 33.3:1 | 2:0.06 |
| X.12 | Fludioxonil | 1:30 | 0.2:6 |
| X.12 | Fludioxonil | 3.3:1 | 0.2:0.06 |
| X.12 | Azoxystrobin | 1:3 | 2:6 |
| X.12 | Azoxystrobin | 3.3:1 | 2:0.6 |
| X.12 | Azoxystrobin | 1:30 | 0.2:6 |
| X.12 | Azoxystrobin | 1:3 | 0.2:0.6 |
| X.12 | Difenoconazole | 1:3 | 2:6 |
| X.12 | Difenoconazole | 3.3:1 | 2:0.6 |
| X.12 | Difenoconazole | 1:30 | 0.2:6 |
| X.12 | Difenoconazole | 1:3 | 0.2:0.6 |
| X.12 | Prothioconazole | 3.3:1 | 2:0.6 |
| X.12 | Prothioconazole | 33.3:1 | 2:0.06 |
| X.12 | Prothioconazole | 1:3 | 0.2:0.6 |
| X.12 | Prothioconazole | 3.3:1 | 0.2:0.06 |
| X.12 | Mefentrifluconazole | 1:3 | 2:6 |
| X.12 | Mefentrifluconazole | 20:1 | 2:0.1 |
| X.12 | Mefentrifluconazole | 1:30 | 0.2:6 |
| X.12 | Mefentrifluconazole | 2:1 | 0.2:0.1 |
| X.12 | Florylpicoxamid | 2:1 | 2:1 |
| X.12 | Florylpicoxamid | 20:1 | 2:0.1 |
| X.12 | Florylpicoxamid | 1:5 | 0.2:1 |
| X.12 | Florylpicoxamid | 2:1 | 0.2:0.1 |
| X.12 | Acibenzolar-S-methyl | 1:10 | 2:20 |
| X.12 | Acibenzolar-S-methyl | 1:3 | 2:6 |
| X.12 | Acibenzolar-S-methyl | 1:100 | 0.2:20 |
| X.12 | Acibenzolar-S-methyl | 1:30 | 0.2:6 |
| X.13 | Pydiflumetofen | 1:30 | 0.2:6 |
| X.13 | Pydiflumetofen | 1:3 | 0.2:0.6 |
| X.13 | Pydiflumetofen | 1:300 | 0.02:6 |
| X.13 | Pydiflumetofen | 1:30 | 0.02:0.6 |
| X.13 | Benzovindiflupyr | 1:10 | 0.2:2 |
| X.13 | Benzovindiflupyr | 1:1 | 0.2:0.2 |
| X.13 | Benzovindiflupyr | 1:100 | 0.02:2 |
| X.13 | Benzovindiflupyr | 1:10 | 0.02:0.2 |
| X.13 | Cyprodinil | 1:100 | 0.2:20 |
| X.13 | Cyprodinil | 2:1 | 0.2:0.1 |
| X.13 | Cyprodinil | 1:1000 | 0.02:20 |
| X.13 | Cyprodinil | 1:5 | 0.02:0.1 |
| X.13 | Fludioxonil | 1:30 | 0.2:6 |
| X.13 | Fludioxonil | 3.3:1 | 0.2:0.06 |
| X.13 | Fludioxonil | 1:300 | 0.02:6 |
| X.13 | Fludioxonil | 1:3 | 0.02:0.06 |
| X.13 | Azoxystrobin | 1:30 | 0.2:6 |
| X.13 | Azoxystrobin | 1:3 | 0.2:0.6 |
| X.13 | Azoxystrobin | 1:300 | 0.02:6 |
| X.13 | Azoxystrobin | 1:30 | 0.02:0.6 |
| X.13 | Difenoconazole | 1:30 | 0.2:6 |
| X.13 | Difenoconazole | 1:3 | 0.2:0.6 |
| X.13 | Difenoconazole | 1:300 | 0.02:6 |
| X.13 | Difenoconazole | 1:30 | 0.02:0.6 |
| X.13 | Prothioconazole | 1:3 | 0.2:0.6 |
| X.13 | Prothioconazole | 3.3:1 | 0.2:0.06 |
| X.13 | Prothioconazole | 1:30 | 0.02:0.6 |
| X.13 | Prothioconazole | 1:3 | 0.02:0.06 |
| X.13 | Mefentrifluconazole | 1:30 | 0.2:6 |
| X.13 | Mefentrifluconazole | 2:1 | 0.2:0.1 |
| X.13 | Mefentrifluconazole | 1:300 | 0.02:6 |
| X.13 | Mefentrifluconazole | 1:5 | 0.02:0.1 |
| X.13 | Florylpicoxamid | 1:5 | 0.2:1 |
| X.13 | Florylpicoxamid | 2:1 | 0.2:0.1 |
| X.13 | Florylpicoxamid | 1:50 | 0.02:1 |
| X.13 | Florylpicoxamid | 1:5 | 0.02:0.1 |
| X.13 | Acibenzolar-S-methyl | 1:100 | 0.2:20 |
| X.13 | Acibenzolar-S-methyl | 1:30 | 0.2:6 |
| X.13 | Acibenzolar-S-methyl | 1:1000 | 0.02:20 |
| X.13 | Acibenzolar-S-methyl | 1:300 | 0.02:6 |
| X.15 | Pydiflumetofen | 1:3 | 2:6 |
| X.15 | Pydiflumetofen | 3.3:1 | 2:0.6 |
| X.15 | Pydiflumetofen | 1:10 | 0.6:6 |
| X.15 | Pydiflumetofen | 1:1 | 0.6:0.6 |
| X.15 | Benzovindiflupyr | 1:1 | 2:2 |
| X.15 | Benzovindiflupyr | 10:1 | 2:0.2 |
| X.15 | Benzovindiflupyr | 1:3.3 | 0.6:2 |
| X.15 | Benzovindiflupyr | 3:1 | 0.6:0.2 |
| X.15 | Cyprodinil | 1:10 | 2:20 |
| X.15 | Cyprodinil | 20:1 | 2:0.1 |
| X.15 | Cyprodinil | 1:33.3 | 0.6:20 |
| X.15 | Cyprodinil | 6:1 | 0.6:0.1 |
| X.15 | Fludioxonil | 1:3 | 2:6 |
| X.15 | Fludioxonil | 33.3:1 | 2:0.06 |
| X.15 | Fludioxonil | 1:10 | 0.6:6 |
| X.15 | Fludioxonil | 10:1 | 0.6:0.06 |
| X.15 | Azoxystrobin | 1:3 | 2:6 |
| X.15 | Azoxystrobin | 3.3:1 | 2:0.6 |
| X.15 | Azoxystrobin | 1:10 | 0.6:6 |
| X.15 | Azoxystrobin | 1:1 | 0.6:0.6 |
| X.15 | Difenoconazole | 1:3 | 2:6 |
| X.15 | Difenoconazole | 3.3:1 | 2:0.6 |
| X.15 | Difenoconazole | 1:10 | 0.6:6 |
| X.15 | Difenoconazole | 1:1 | 0.6:0.6 |
| X.15 | Prothioconazole | 3.3:1 | 2:0.6 |
| X.15 | Prothioconazole | 33.3:1 | 2:0.06 |
| X.15 | Prothioconazole | 1:1 | 0.6:0.6 |
| X.15 | Prothioconazole | 10:1 | 0.6:0.06 |
| X.15 | Mefentrifluconazole | 1:3 | 2:6 |
| X.15 | Mefentrifluconazole | 20:1 | 2:0.1 |
| X.15 | Mefentrifluconazole | 1:10 | 0.6:6 |
| X.15 | Mefentrifluconazole | 6:1 | 0.6:0.1 |
| X.15 | Florylpicoxamid | 2:1 | 2:1 |
| X.15 | Florylpicoxamid | 20:1 | 2:0.1 |
| X.15 | Florylpicoxamid | 1:1.6 | 0.6:1 |
| X.15 | Florylpicoxamid | 6:1 | 0.6:0.1 |
| X.15 | Acibenzolar-S-methyl | 1:10 | 2:20 |
| X.15 | Acibenzolar-S-methyl | 1:3 | 2:6 |
| X.16 | Pydiflumetofen | 10:1 | 6:0.6 |
| X.16 | Pydiflumetofen | 1:6 | 1:6 |
| X.16 | Pydiflumetofen | 1.6:1 | 1:0.6 |
| X.16 | Benzovindiflupyr | 3:1 | 6:2 |
| X.16 | Benzovindiflupyr | 30:1 | 6:0.2 |
| X.16 | Benzovindiflupyr | 1:2 | 1:2 |
| X.16 | Benzovindiflupyr | 5:1 | 1:0.2 |
| X.16 | Cyprodinil | 1:3.3 | 6:20 |
| X.16 | Cyprodinil | 60:1 | 6:0.1 |
| X.16 | Cyprodinil | 1:20 | 1:20 |
| X.16 | Cyprodinil | 10:1 | 1:0.1 |
| X.16 | Fludioxonil | 1:1 | 6:6 |
| X.16 | Fludioxonil | 100:1 | 6:0.06 |
| X.16 | Fludioxonil | 1:6 | 1:6 |
| X.16 | Fludioxonil | 16.6:1 | 1:0.6 |
| X.16 | Azoxystrobin | 1:1 | 6:6 |
| X.16 | Azoxystrobin | 10:1 | 6:0.6 |
| X.16 | Azoxystrobin | 1:6 | 1:6 |
| X.16 | Azoxystrobin | 1.6:1 | 1:0.6 |
| X.16 | Difenoconazole | 1:1 | 6:6 |
| X.16 | Difenoconazole | 10:1 | 6:0.6 |
| X.16 | Difenoconazole | 1:6 | 1:6 |
| X.16 | Difenoconazole | 1.6:1 | 1:0.6 |
| X.16 | Prothioconazole | 10:1 | 6:0.6 |
| X.16 | Prothioconazole | 100:1 | 6:0.06 |
| X.16 | Mefentrifluconazole | 1:1 | 6:6 |
| X.16 | Mefentrifluconazole | 60:1 | 6:0.1 |
| X.16 | Mefentrifluconazole | 1:6 | 1:6 |
| X.16 | Mefentrifluconazole | 10:1 | 1:0.1 |
| X.16 | Florylpicoxamid | 6:1 | 6:1 |
| X.16 | Florylpicoxamid | 60:1 | 6:0.1 |
| X.16 | Florylpicoxamid | 1:1 | 1:1 |
| X.16 | Florylpicoxamid | 10:1 | 1:0.1 |
| X.16 | Acibenzolar-S-methyl | 1:3.3 | 6:20 |
| X.16 | Acibenzolar-S-methyl | 1:1 | 6:6 |
| X.14 | Pydiflumetofen | 3.3:1 | 20:6 |
| X.14 | Pydiflumetofen | 33.3:1 | 20:0.6 |
| X.14 | Pydiflumetofen | 1:1 | 6:6 |
| X.14 | Pydiflumetofen | 10:1 | 6:0.6 |
| X.14 | Benzovindiflupyr | 10:1 | 20:2 |
| X.15 | Acibenzolar-S-methyl | 1:33.3 | 0.6:20 |
| X.15 | Acibenzolar-S-methyl | 1:10 | 0.6:6 |
| X.14 | Benzovindiflupyr | 100:1 | 20:0.2 |
| X.14 | Benzovindiflupyr | 3:1 | 6:2 |
| X.14 | Benzovindiflupyr | 30:1 | 6:0.2 |
| X.14 | Cyprodinil | 1:1 | 20:20 |
| X.14 | Cyprodinil | 200:1 | 20:0.1 |
| X.14 | Cyprodinil | 1:3.3 | 6:20 |
| X.14 | Cyprodinil | 60:1 | 6:0.1 |
| X.14 | Fludioxonil | 3.3:1 | 20:6 |
| X.14 | Fludioxonil | 333.3:1 | 20:0.06 |
| X.14 | Fludioxonil | 1:1 | 6:6 |
| X.14 | Fludioxonil | 100:1 | 6:0.06 |
| X.14 | Azoxystrobin | 3.3:1 | 20:6 |
| X.14 | Azoxystrobin | 33.3:1 | 20:0.6 |
| X.14 | Azoxystrobin | 1:1 | 6:6 |
| X.14 | Azoxystrobin | 10:1 | 6:0.6 |
| X.14 | Difenoconazole | 3.3:1 | 20:6 |
| X.14 | Difenoconazole | 33.3:1 | 20:0.6 |
| X.14 | Difenoconazole | 1:1 | 6:6 |
| X.14 | Difenoconazole | 10:1 | 6:0.6 |
| X.14 | Prothioconazole | 33.3:1 | 20:0.6 |
| X.14 | Prothioconazole | 333.3:1 | 20:0.06 |
| X.14 | Prothioconazole | 10:1 | 6:0.6 |
| X.14 | Prothioconazole | 100:1 | 6:0.06 |
| X.14 | Mefentrifluconazole | 3.3:1 | 20:6 |
| X.14 | Mefentrifluconazole | 200:1 | 20:0.1 |
| X.14 | Mefentrifluconazole | 1:1 | 6:6 |
| X.14 | Mefentrifluconazole | 60:1 | 6:0.1 |
| X.14 | Florylpicoxamid | 20:1 | 20:1 |
| X.14 | Florylpicoxamid | 200:1 | 20:0.1 |
| X.14 | Florylpicoxamid | 6:1 | 6:1 |
| X.14 | Florylpicoxamid | 60:1 | 6:0.1 |
| X.14 | Acibenzolar-S-methyl | 1:1 | 20:20 |
| X.14 | Acibenzolar-S-methyl | 3.3:1 | 20:6 |
| X.14 | Acibenzolar-S-methyl | 1:3.3 | 6:20 |
| X.14 | Acibenzolar-S-methyl | 1:1 | 6:6 |

1. A fungicidal composition comprising components (A) and (B) as active ingredients, wherein component (A) is a compound of formula (I): 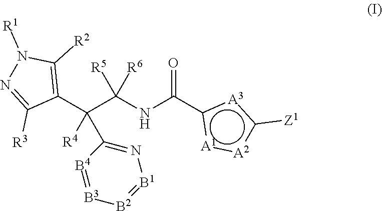
wherein
R¹ is selected from C₁-C₄-alkyl;
R² is selected from hydrogen, halogen, or C₁-C₄-alkyl;
R³ is selected from hydrogen or C₁-C₄-alkyl;
R⁴ is selected from C₁-C₄-alkyl;
R⁵ and R⁶ are independently selected from hydrogen or C₁-C₄-alkyl;
A¹, A² and A³ are independently selected from CR⁷, N, NR⁸, O, or S, with the proviso that at least one of A¹, A² and A³ is selected from N, O or S, and that no more than one of A¹, A² and A³ is O or S;
R⁷ and R⁸ are independently selected from hydrogen, or C₁-C₄-alkyl;
B¹ is CR⁹ or N, B² is CR¹⁰ or N, B³ is CR¹¹ or N, B⁴ is CR¹² or N, with the proviso that only one of B¹, B², B³, and B⁴ is N;
R⁹, R¹⁰, R¹¹ and R¹² are independently selected from hydrogen, halogen, cyano, C₁-C₄-alkyl, C₁-C₄-alkoxy, C₁-C₄-haloalkyl, C₁-C₄-haloalkoxy, C₁-C₄-alkoxy-C₁-C₄-alkyl, C₁-C₄-alkoxycarbonyl, C₁-C₄-alkylcarbonyl, N—C₁-C₄ alkoxy-C₁-C₄-alkyl-carbonimidoyl, N-hydroxy-C₁-C₄-alkyl-carbonimidoyl, or C₃-C₆-ycloalkyl, wherein said C₃-C₆-cycloalkyl is unsubstituted or substituted by lor 2 substituents independently selected from halogen, cyano, C₁-C₄-alkyl, C₁-C₄-haloalkyl, or C₁-C₄-alkoxy; and
Z¹ is selected from C₁-C₄-alkyl, phenyl, pyridyl, or C₃-C₆-cycloalkyl, wherein any of said phenyl, pyridyl and C₃-C₆-cycloalkyl are unsubstituted or substituted by 1 or 2 substituents independently selected from halogen, cyano, C₁-C₄-alkyl, C₁-C₄-haloalkyl, C₁-C₄-alkoxy, or C₁-C₄-haloalkoxy;
or salt or N-oxide thereof,
and
component (B) is a compound selected from the group consisting of: pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidin, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethyl-cyclobutyl)-5-methyl-thiazole-4-carboxamide, 2-[(2,6-difluoro-4-pyridyl)-(tetrahydrofuran-3-carbonyl) amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

2. The fungicidal composition according claim 1, wherein component (A) is a compound selected from:
N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-(1-methylpyrazol-4-yl)-2-(2-pyridyl)propyl]isoxazole-3-carboxamide (X.03), N-[(2S)-2-(6-chloro-4-methoxy-2-pyridyl)-2-(1-methylpyrazol-4-yl)propyl]-3-(2,4-difluorophenyl)isoxazole-5-carboxamide (X.04), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05), N-[2-(5-chloro-2-pyridyl)-2-(1-methylpyrazol-4-yl)propyl]-5-(2,4-difluorophenyl)-1,3,4-thiadiazole-2-carboxamide (X.06), N-[(2R)-2-(6-chloro-4-methoxy-2-pyridyl)-2-(1-methylpyrazol-4-yl)propyl]-3-(2,4-difluorophenyl)isoxazole-5-carboxamide (X.07), 5-(2,4-difluorophenyl)-N-[2-(1-methylpyrazol-4-yl)-2-(5-methyl-2-pyridyl)propyl]isoxazole-3-carboxamide (X.08), N-[2-(6-chloro-2-pyridyl)-2-(1-methylpyrazol-4-yl)propyl]-5-(2,4-dichlorophenyl)isoxazole-3-carboxamide (X.09), methyl-6-[2-[[5-(2,4-difluorophenyl)isoxazole-3-carbonyl]amino]-1-methyl-1-(1-methylpyrazol-4-yl)ethyl]pyridine-3-carboxylate (X.10), N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), N-[2-(6-cyano-2-pyridyl)-2-(1-methylpyrazol-4-yl)propyl]-5-(3,5-difluoro-2-pyridyl)-1,3,4-thiadiazole-2-carboxamide (X.14), N-[2-(6-chloro-2-pyridyl)-2-(1-methylpyrazol-4-yl)propyl]-5-(3,5-difluoro-2-pyridyl)-1,3,4-thiadiazole-2-carboxamide (X.15), or N-[2-(6-chloro-2-pyridyl)-2-(1-methylpyrazol-4-yl)propyl]-5-(2,6-difluoro-3-pyridyl)-1,3,4-thiadiazole-2-carboxamide (X.16)

3. The fungicidal composition according to claim 1, wherein component (A) is:
N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-(1-methylpyrazol-4-yl)-2-(2-pyridyl)propyl]isoxazole-3-carboxamide (X.03), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05),
N-[2-(5-chloro-2-pyridyl)-2-(1-methylpyrazol-4-yl)propyl]-5-(2,4-difluorophenyl)-1,3,4-thiadiazole-2-carboxamide (X.06), N-[(2R)-2-(6-chloro-4-methoxy-2-pyridyl)-2-(1-methylpyrazol-4-yl)propyl]-3-(2,4-difluorophenyl)isoxazole-5-carboxamide (X.07), methyl-6-[2-[[5-(2,4-difluorophenyl)isoxazole-3-carbonyl]amino]-1-methyl-1-(1-methylpyrazol-4-yl)ethyl]pyridine-3-carboxylate (X.10),
N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), or N-[2-(6-chloro-2-pyridyl)-2-(1-methylpyrazol-4-yl)propyl]-5-(3,5-difluoro-2-pyridyl)-1,3,4-thiadiazole-2-carboxamide (X.15).

4. The fungicidal composition according to claim 1, wherein component (A) is:
N-[2-(6-chloro-2-pyridyl)-2-(1-methylpyrazol-4-yl)propyl]-5-(2,4-difluorophenyl)isoxazole-3-carboxamide (X.01), N-[2-(6-cyano-2-pyridyl)-2-(1-methylpyrazol-4-yl)propyl]-5-(2,4-difluorophenyl)isoxazole-3-carboxamide (X.02), 5-(2,4-difluorophenyl)-N-[2-(1-methylpyrazol-4-yl)-2-(2-pyridyl)propyl]isoxazole-3-carboxamide (X.03), 5-(2,4-difluorophenyl)-N-[2-[6-[(E)-N-methoxy-C-methyl-carbonimidoyl]-2-pyridyl]-2-(1-methylpyrazol-4-yl)propyl]isoxazole-3-carboxamide (X.05),
N-[2-(5-chloro-2-pyridyl)-2-(1-methylpyrazol-4-yl)propyl]-5-(2,4-difluorophenyl)-1,3,4-thiadiazole-2-carboxamide (X.06), N-[2-(6-chloro-2-pyridyl)-2-(1-methylpyrazol-4-yl)propyl]-5-(3,5-difluoro-2-pyridyl)isoxazole-3-carboxamide (X.11), N-[2-(6-chloro-2-pyridyl)-2-(1-methylpyrazol-4-yl)propyl]-3-(2,4-difluorophenyl)-1,2,4-oxadiazole-5-carboxamide (X.12), N-[2-(6-chloro-2-pyridyl)-2-(1-methylpyrazol-4-yl)propyl]-5-(2,4-difluorophenyl)-1,3,4-thiadiazole-2-carboxamide (X.13), or
N-[2-(6-chloro-2-pyridyl)-2-(1-methylpyrazol-4-yl)propyl]-5-(3,5-difluoro-2-pyridyl)-1,3,4-thiadiazole-2-carboxamide (X.15).

5. The fungicidal composition according to claim 1, wherein component (B) is a compound selected from the group consisting of pydiflumetofen, benzovindiflupyr, bixafen, fluxapyroxad, isopyrazam, penthiopyrad, sedaxane, boscalid, fluopyram, thifluzamide, pyraziflumid, isoflucypram, inpyrfluxam, fluindapyr, cyclobutrifluram, pyraclostrobin, trifloxystrobin, azoxystrobin, metyltetraprole, fenpicoxamid, florylpicoxamid, metarylpicoxamid, fluazinam, fenpropidin, fenhexamid, cyproconazole, difenoconazole, metconazole, penconazole, propiconazole, tebuconazole, tetraconazole, prothioconazole, mefentrifluconazole, flufenoxadiazam, ipflufenoquin, quinofumelin, metalaxyl-M, cyprodinil, pyrimethanil, mancozeb, copper-compounds (different salts), sulphur, folpet, chlorothalonil, dithianon, proquinazid, fludioxonil, metrafenone, oxathiapiprolin, fluoxapiprolin, acibenzolar-S-methyl, phosphorous acid, cyflufenamid, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

6. The fungicidal composition according to n claim 1, wherein component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, acibenzolar-S-methyl, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, 2-[cyano-(2,6-difluoro-4-pyridyl)-amino]-5-methyl-N-spiro[3.4]octan-3-yl-thiazole-4-carboxamide, 2-[cyano-(2,6-difluoro-4-pyridyl)-amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

7. The fungicidal composition according to claim 1, wherein component (B) is a compound selected from the group consisting of pydiflumetofen, benzovindiflupyr, azoxystrobin, florylpicoxamid, difenoconazole, prothioconazole, mefentrifluconazole, cyprodinil, fludioxonil, and acibenzolar-S-methyl.

8. The fungicidal composition according to claim 1, wherein the weight ratio of component (A) to component (B) is from 20:1 to 1:40.

9. The fungicidal composition according to claim 1, wherein the weight ratio of component (A) to component (B) is from 12:1 to 1:25.

10. The fungicidal composition according to claim 1, wherein the weight ratio of component (A) to component (B) is from 5:1 to 1:15.

11. The fungicidal composition according to claim 1, wherein the weight ratio of component (A) to component (B) is from 2:1 to 1:5.

12. The fungicidal composition according to claim 1, wherein the composition may comprise an additional active ingredient component (C), which is different to component (B), and is selected from the group consisting of pydiflumetofen, benzovindiflupyr, bixafen, fluxapyroxad, isopyrazam, penflufen, penthiopyrad, sedaxane, boscalid, fluopyram, thifluzamide, pyraziflumid, isoflucypram, inpyrfluxam, isofetamid, fluindapyr, cyclobutrifluram, fluoxastrobin, fenamidone, mandestrobin, picoxystrobin, pyraclostrobin, famoxadone, kresoxim-methyl, trifloxystrobin, azoxystrobin, metyltetraprole, amisulbrom, cyazofamid, fenpicoxamid, florylpicoxamid, metarylpicoxamid, ametoctradin, fluazinam, fentin hydroxide, silthiofam, fenpropimorph, fenpropidine, spiroxamine, fenhexamid, imazalil, pyrisoxazole, bromuconazole, cyproconazole, difenoconazole, epoxiconazole, flutriafol, hexaconazole, ipconazole, metconazole, myclobutanil, penconazole, propiconazole, tebuconazole, tetraconazole, triticonazole, prothioconazole, fluoxytioconazole, mefentrifluconazole, flufenoxadiazam, ipflufenoquin, quinofumelin, metalaxyl-M, cyprodinil, pyrimethanil, kasugamycin, mancozeb, copper fungicides, sulphur, zinc thiazole, captan, folpet, chlorothalonil, dithianon, quinoxyfen, proquinazid, fludioxonil, iprodione, procymidone, thiabendazole, zoxamide, metrafenone, fluopicolide, propamocarb, oxathiapiprolin, fluoxapiprolin, acibenzolar-S-methyl, isotianil, phosphorous acid, cyflufenamid, tebufloquin, picarbutrazox, tricyclazole, N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate, 2-[cyano-(2,6-difluoro-4-pyridyl)amino]-5-methyl-N-spiro[3.4]octan-3-yl-thiazole-4-carboxamide, 2-[cyano-(2,6-difluoro-4-pyridyl)amino]-N-(2,2-dimethylcyclobutyl)-5-methyl-thiazole-4-carboxamide, 2-[cyano-(2,6-difluoro-4-pyridyl)amino]-N-hexyl-5-methyl-thiazole-4-carboxamide, 2-[(2,6-difluoro-4-pyridyl)-(2-methoxyacetyl)amino]-N-(2,2-dimethylcyclobutyl)-5-methyl-thiazole-4-carboxamide, 2-[(2,6-difluoro-4-pyridyl)-(tetrahydropyran-4-carbonyl)amino]-N-(2,2-dimethylcyclobutyl)-5-methyl-thiazole-4-carboxamide, 2-[(2,6-difluoro-4-pyridyl)-(oxetane-3-carbonyl)amino]-N-(2,2-dimethylcyclobutyl)-5-methyl-thiazole-4-carboxamide, 2-[(2,6-difluoro-4-pyridyl)-(tetrahydrofuran-3-carbonyl)amino]-N-(2,2-dimethylcyclobutyl)-5-methyl-thiazole-4-carboxamide, 2-[acetyl-(2,6-difluoro-4-pyridyl)amino]-N-(2,2-dimethylcyclobutyl)-5-methyl-thiazole-4-carboxamide, 2-[(2,6-difluoro-4-pyridyl)-(2-methylpropanoyl)amino]-N-(2,2-dimethylc-yclobutyl)-5-methyl-thiazole-4-carboxamide, TAEGRO® (i.e, Bacillus amyloliquefaciens strain FZB24), Melaluca alternifolia oil (an extract of the tea tree plant Melaluca alternifolia (commercially available as Timorex Gold®, which is a broad-spectrum botanical biofungicide)), Reynoutria sachalinensis extract (commercially available as REGALIA®), a plant extract based on the extract of Quillaja saponaria Molina (commercially available as BOTRISTOP®), and Aureobasidin A.

13. The fungicidal composition according to claim 1, wherein said composition further comprises an agriculturally acceptable carrier and, optionally, a surfactant and/or formulation adjuvants.

14. A method of controlling or preventing phytopathogenic diseases, especially phytopathogenic fungi, on useful plants or on propagation material thereof, which comprises applying to the useful plants, the locus thereof or propagation material thereof the fungicidal composition comprising component (A) and component (B) as defined in claim 1.

15. The method according to claim 14, wherein the components (A) and (B) are applied in a sequential manner.
